(19) United States
(12) Patent Application Publication
(10) Pub. No.: US 2017/0191091 A1
(43) Pub. Date: Jul. 6, 2017

(54) RECOMBINANT CELLS PRODUCING ITACONIC ACID AND METHYL ESTERS THEREOF

(71) Applicant: DSM IP Assets B.V., Heerlen (NL)

(72) Inventors: Zheng ZHAO, Echt (NL); Bernard MEIJRINK, Echt (NL); Robertus Antonius Mijndert VAN DER HOEVEN, Echt (NL); Liang WU, Echt (NL); Johannes Andries ROUBOS, Echt (NL); Marina VAN GELDER, Echt (NL)

(21) Appl. No.: 15/314,494

(22) PCT Filed: May 28, 2015
(86) PCT No.: PCT/EP2015/061884

§ 371 Date: Nov. 28, 2016

Related U.S. Application Data
(60) Provisional application No. 62/004,789, filed on May 29, 2014.

(51) Int. Cl. C12P 7/62; C12N 15/52; C12P 7/46

(52) U.S. Cl. CPC C12P 7/62; C12P 7/46; C12N 15/52

(57) ABSTRACT

The present invention relates to a recombinant cell which is capable of producing one or more of itaconic acid, 4-methyl itaconate or 1-methyl itaconate, wherein the said recombinant microorganism has been modified in its genome such that it results in a deficiency in the production of a trans-aconitate methyltransferase. A recombinant yeast cell which is capable of producing itaconic acid and which overexpresses: —a nucleic acid encoding a polypeptide having cis-aconitate decarboxylase activity; and —one or more nucleic acids encoding polypeptides which separately or together catalyze a reaction towards acetyl CoA, wherein the said recombinant microorganism has been modified in its genome such that it results in a deficiency in the production of a trans-aconitate methyltransferase. These recombinant yeast cells may be used in processes for the production of itaconic acid, 4-methyl itaconate or 1-methyl itaconate.

### FIELD OF THE INVENTION

The present invention relates to a recombinant microorganism capable of producing itaconic acid and/or itaconate methylester and to a process for the production of itaconic acid and/or itaconate methylester by use of such a cell. The invention further relates to a fermentation broth comprising itaconic acid and/or itaconate methylester obtainable by such a process.

### BACKGROUND TO THE INVENTION

Itaconic acid, an essential precursor to various products (e.g., acrylic fibers, rubbers, artificial diamonds, and lens), is in high demand in the chemical industry. Conventionally, itaconic acid is isolated from the filamentous fungus Aspergillus terreus. In addition, itaconic acid esters may be key intermediates for both commodity and specialty chemicals. The itaconic acid mono-methyl esters, i.e. 4-methyl itaconate and 1-methyl itaconate are particularly interesting in this respect.

Recently, Aspergillus niger has been genetically modified to produce itaconic acid (WO2009014437, WO2009104958) by overexpressing cis-aconitate decarboxylase (CAD) and/or a putative itaconic acid transporter. However, Aspergilli are less suitable for industrial production of itaconic acid due to its filamentous morphology, leading to oxygen transfer problems in large scale bioreactors.

E. coli has also been genetically modified to produce itacionic acid (US2010285546) by overexpressing CAD in combination with reduced isocitrate dehydrogenase (ICD) activity. This approach is problematic, however, since E. coli, and prokaryotes in general, are not tolerant to low pH. In a high pH fermentation (e.g. about pH7 which is optimal for E. coli), titration is needed to keep pH constant and this leads to the formation of itaconic salts instead of the acid. This in turn leads to increased DSP costs since recovery of the acid from the salt is more complex, as compared with a low pH fermentation process, where the acid can be directly recovered from the fermentation broth by crystallization.

More recently, a non-filamentous yeast, Yarrowia lipolytica, has been genetically modified to produce itaconic acid on glycerol (US20110053232). However, the modified Y. lipolytica does not produce significant amounts of itaconic acid on sugar, one of the most commonly available renewable feedstocks.

Accordingly, there is a need to further improve itaconic acid production processes based on fermentation from sugar at low pH so that economically viable, large scale production may be achieved in industrial bioreactors.

### SUMMARY OF THE INVENTION

The present invention is based on the unexpected identification of a recombinant cells, i.e. a genetically modified cells, that may produce itaconic acid and/or an ester of itaconic acid. These cells may be yeast cells. The advantage of yeast is that it is tolerant to low pH and is not filamentous, which allows for the optimal process conditions to produce itaconic acid and/or itaconic acid methyl ester. Cells of the invention may preferentially produce itaconic acid with reduced amounts of methyl esters.

Accordingly, the invention relates to a recombinant cell which is capable of producing one or more of itaconic acid, 4-methyl itaconate or 1-methyl itaconate, wherein the said recombinant microorganism has been modified in its genome such that it results in a deficiency in the production of a trans-aconitate methyltransferase.

The invention also relates to a recombinant yeast cell which is capable of producing itaconic acid and which overexpresses:
a nucleic acid encoding a polypeptide having cis-aconitate decarboxylase activity; and
a nucleic acid encoding a polypeptide which catalyzes a reaction towards acetyl CoA,

wherein the said recombinant microorganism has been modified in its genome such that it results in a deficiency in the production of a trans-aconitate methyltransferase.

Recombinant cells of the invention may be used in processes for the production of itaconic acid and/or an ester of itaconic acid.

Thus the invention provides:
a process for the production of 4-methyl itaconate or 1-methyl itaconate, which process comprises fermenting a recombinant cell according of the invention in a suitable fermentation medium, wherein 4-methyl itaconate or 1-methyl itaconate is produced;
a process for the production of itaconic acid or an ester of itaconic acid, which process comprises fermenting a yeast cell according to the invention in a suitable fermentation medium, wherein the itaconic acid or ester of itaconic acid is produced.

The itaconic acid or ester of itaconic acid may be further converted into a pharmaceutical, cosmetic, food, feed or chemical product.

Also, the invention provides a fermentation broth comprising itaconic acid and/or an ester of itaconic acid obtainable by a process of the invention.

### BRIEF DESCRIPTION OF THE DRAWINGS

FIG. 1a-d sets out metabolic pathways allowing the production of itaconic acid. Numbered reactions shows enzymes which may be overexpressed as follows. Reaction (1): pyruvate carboxylase. Conversion of cytosolic pyruvate and bicarbonate to oxaloacetate. Reaction (2): mitochondrial oxaloacetate transporter. Transportation of cytosolic oxaloacetate to mitochondrial oxaloacetate. Reaction (3): mitochondrial membrane citrate transporter. Transportation of mitochondrial citrate to cytosolic citrate and vice versa. Reaction (4): Aconitase. Conversion of citrate to aconitate. Reaction (5): cis-aconitate decarboxylase. Conversion of cis-aconitate to itaconate. Reaction (6): Itaconic acid transporter. transportation of cytosolic itaconate to extracellular itaconic acid. Reaction (7): citrate synthase. conversion of cytosolic oxaloacetate and acetyl coenzyme-A to citrate. Reaction (8): acetylating acetaldehyde dehydrogenase. conversion of cytosolic acetaldehyde, NAD, and coenzyme-A to acetyl-coenzyme-A and NADH. Reaction (9): Phosphoketolase. Conversion of xylulose 5-phosphate to acetyl phosphate, glceraldehyde 3-phosphate, and water; or conversion of fructose 6-phosphate to acetyl phosphate, erythrose 4-phosphate, and water. Reaction (10): phosphate acetyltransferase. Conversion of coenzyme-A and acetyl phosphate to acetyl coenzyme-A and phosphate. Reaction (11): ATP:acetate phosphotransferase. Conversion of acetate and ATP to acetyl phosphate and ADP. The reactions highlighted by thicker arrow are the reactions expected to be relevant for conversion from glucose to itaonic acid and/or itaconate.

FIG. 2 sets out metabolic pathways allowing the production of esters of itaconic acid.

FIG. 3 sets out a schematic diagram of the construction of a TMT1 knock-out strain.

### DESCRIPTION OF THE SEQUENCE LISTING

A description of the sequences is set out in Table 4, 5 and 6. Sequences described herein may be defined with reference to the sequence listing or with reference to the database accession numbers also set out in Table 4, 5 and 6.

### DETAILED DESCRIPTION OF THE INVENTION

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

In Aspergillus terreus, itaconic acid is synthesized from cis-aconitate, which is an intermediate of the tricarboxylic acid cycle. The enzyme responsible for converting cis-aconitate to itaconic acid is cis-aconitate decarboxylase. We have shown that this enzyme may be overexpressed in recombinant cells so that cells which do not typically produce itaconic acid may do so. Overexpression of one or more enzymes catalysing reactions to acetyl-CoA can further improve the amount of itaconic acid product. Also, such recombinant cells may produce an ester of itaconic acid by overexpressing one or more enzymes leading to the production of such an ester.

Overexpression in the context of this invention indicates that a given nucleic acid sequence and/or amino acid sequence is expressed to a greater degree in a recombinant cell of the invention than a reference cell, which may typically be a corresponding wild type cell (i.e. a wild type cell of the same species). A nucleic acid and/or polypeptide may be overexpressed in the sense that a nucleic acid and/or polypeptide expressed in the reference cell is expressed to a greater degree in a recombinant cell of the invention (the reference cell may not express the nucleic acid and/or polypeptide at all). Overexpression may occur, for example, via overexpression of a nucleic acid and/or polypeptide which is endogenous (or homologous) to the reference cell. Overexpression may occurs, for example, via overexpression of a nucleic acid and/or polypeptide which is exogenous (or heterologous) to the reference cell. That is to say, overexpression may occur, for example, via overexpression of a nucleic acid and/or polypeptide which is natively occurs in the reference cell. Overexpression may occur, for example, via overexpression of a nucleic acid and/or polypeptide which is not present or not expressed at all in the reference cell.

A recombinant cell of the invention may overexpress at least one an exogenous nucleic acid and/or polypeptide and overexpress at least one endogenous nucleic acid and/or polypeptide. Critically though, a recombinant cell of the invention is been modified in its genome such that it results in a deficiency in the production of a trans-aconitate methyltransferase.

References herein to carboxylic acids or carboxylates, e.g. itaconic acid/itaconate, should be understood to include the protonated carboxylic acid (free acid), the corresponding carboxylate (its conjugated base) as well as a salt thereof, unless specified otherwise.

According to the invention, there is provided a recombinant cell, typically one which is capable of producing one or more of itaconic acid, 4-methyl itaconate or 1-methyl itaconate, wherein the said recombinant cell has been modified in its genome such that it results in a deficiency in the production of a trans-aconitate methyltransferase. Such a cell may be a yeast cell, such as a Saccharomyces cerevisiae cell.

According to this invention, there is thus provided a recombinant yeast comprising one or more nucleotide sequence(s) encoding (or, optionally, overexpressing):

a polypeptide having cis-aconitate decarboxylase activity; and

a genetic modification leading to an increase in flux towards acetyl-CoA,

wherein the said recombinant cell has been modified in its genome such that it results in a deficiency in the production of a trans-aconitate methyltransferase.

According to this invention, elevated levels of itaconic acid are achieved and the amount of production of itaconate methyl ester may be reduced by increasing combinations of various metabolic reactions rates for the production of one or more of the precursors, including, cis-aconitate, citrate, oxaloacetate, acetyl-Coenzyme-A, and acetyl-phosphate. That is to say, nucleic acid sequences encoding polypeptides carrying out such reactions may be overexpressed.

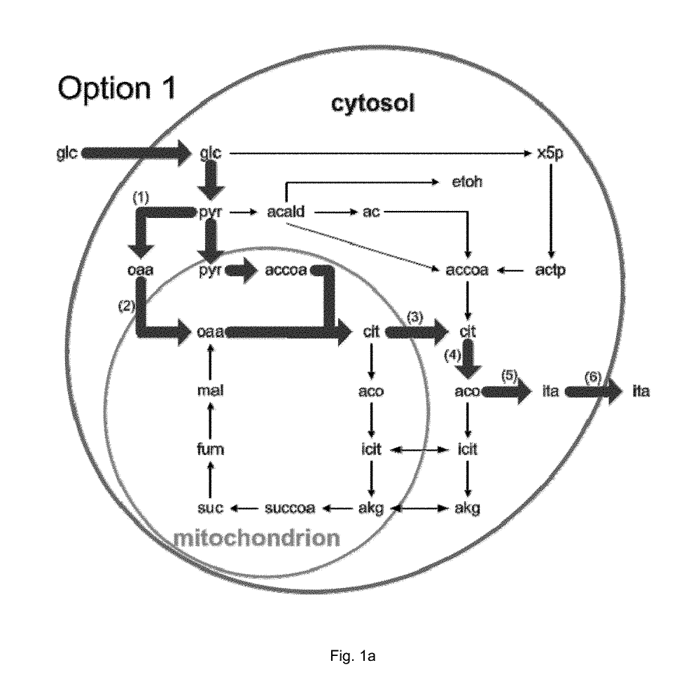

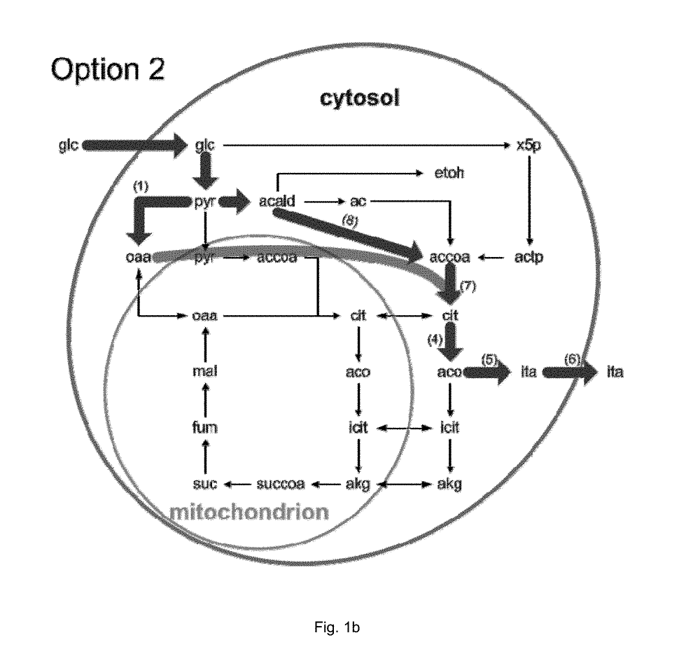

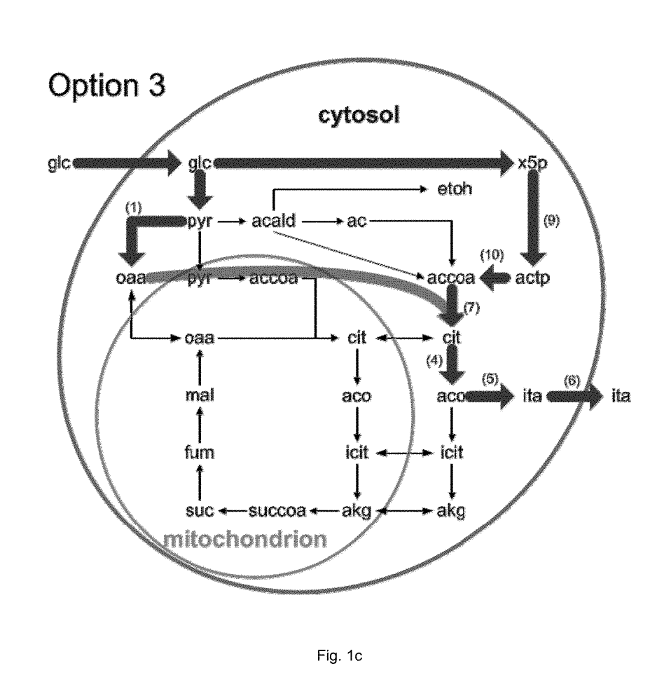

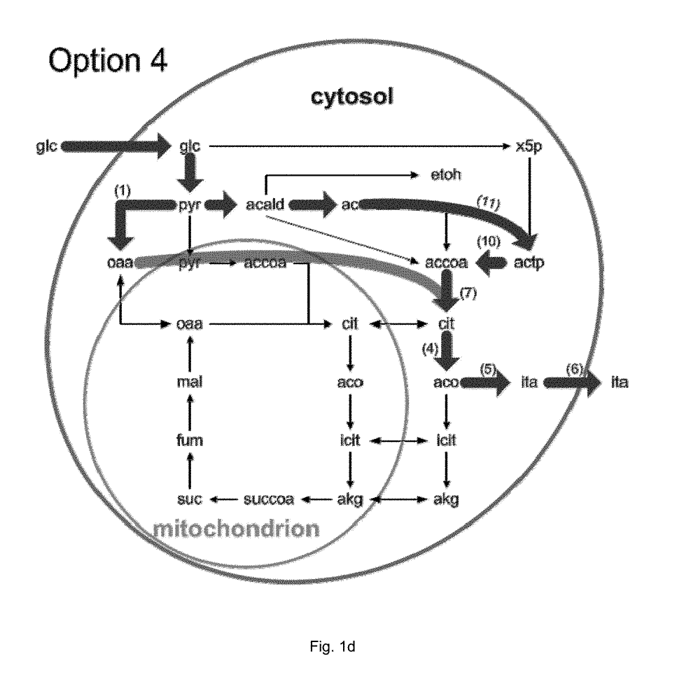

Accordingly, combinations of two or more of the following reactions may be organized into one or more metabolic pathways (the following numbering follows that set out in FIG. 1a-d):

Reaction (1): pyruvate carboxylase. Conversion of cytosolic pyruvate and bicarbonate to oxaloacetate.

Reaction (2): mitochondrial oxaloacetate transporter. Transportation of cytosolic oxaloacetate to mitochondrial oxaloacetate.

Reaction (3): mitochondrial membrane citrate transporter. Transportation of mitochondrial citrate to cytosolic citrate and vice versa.

Reaction (4): Aconitase. Conversion of citrate to aconitate.

Reaction (5): cis-aconitate decarboxylase. Conversion of cis-aconitate to itaconate.

Reaction (6): Itaconic acid transporter. transportation of cytosolic itaconate to extracellular itaconic acid.

Reaction (7): citrate synthase. conversion of cytosolic oxaloacetate and acetyl coenzyme-A to citrate.

Reaction (8): acetylating acetaldehyde dehydrogenase. conversion of cytosolic acetaldehyde, NAD, and coenzyme-A to acetyl-coenzyme-A and NADH.

Reaction (9): Phosphoketolase. Conversion of xylulose 5-phosphate to acetyl phosphate, glceraldehyde 3-phosphate, and water; or conversion of fructose 6-phosphate to acetyl phosphate, erythrose 4-phosphate, and water.

Reaction (10): phosphate acetyltransferase. Conversion of coenzyme-A and acetyl phosphate to acetyl coenzyme-A and phosphate. This enzyme may be referred to as acetyl-CoA:Pi acetyltransferase or acetyl-CoA:phosphate acetyltransferase.

Reaction (11): ATP:acetate phosphotransferase. Conversion of acetate and ATP to acetyl phosphate and ADP.

Preferred combinations are:

A. Reaction (1), (2), (3), (4), (5) and (6)—see FIG. 1a.

B. Reaction (1), (8), (7), (4), (5) and (6)—see FIG. 1b.

C. Reaction (1), (9), (10), (7), (4), (5) and (6)—see FIG. 1c.

D. Reaction (1), (11), (10), (7), (4), (5) and (6)—see FIG. 1d.

Any suitable sequence nucleic acid sequence encoding a polypeptide carrying out the stated reaction may be used in the invention. Examples include:

Reaction (1): SEQ ID NO: 25 or a sequence having at least 50% sequence identity thereto (or at least 60%, at least 70%, at least 75%, at least 80%, at least 90%, at least 95%, at least 98% or at least 99% sequence identity thereto).

Reaction (2): SEQ ID NO: 23 or a sequence having at least 50% sequence identity thereto (or at least 60%, at least 70%, at least 75%, at least 80%, at least 90%, at least 95%, at least 98% or at least 99% sequence identity thereto).

Reaction (3): SEQ ID NO: 21 or 47 or a sequence having at least 50% sequence identity to either of said sequences (or at least 60%, at least 70%, at least 75%, at least 80%, at least 90%, at least 95%, at least 98% or at least 99% sequence identity thereto).

Reaction (4): SEQ ID NO: 15, 17 or 19 or a sequence having at least 50% sequence identity to any of said sequences (or at least 60%, at least 70%, at least 75%, at least 80%, at least 90%, at least 95%, at least 98% or at least 99% sequence identity thereto).

Reaction (5): SEQ ID NO: 7, 9, 11 or 13 or a sequence having at least 50% sequence identity to any of said sequences (or at least 60%, at least 70%, at least 75%, at least 80%, at least 90%, at least 95%, at least 98% or at least 99% sequence identity thereto).

Reaction (6): SEQ ID NO: 1, 3 or 5 or a sequence having at least 50% sequence identity to any of said sequences (or at least 60%, at least 70%, at least 75%, at least 80%, at least 90%, at least 95%, at least 98% or at least 99% sequence identity thereto).

Reaction (7): SEQ ID NO: 27, 29 or 31 or a sequence having at least 50% sequence identity to any of said sequences (or at least 60%, at least 70%, at least 75%, at least 80%, at least 90%, at least 95%, at least 98% or at least 99% sequence identity thereto).

Reaction (8): SEQ ID NO: 33 or a sequence having at least 50% sequence identity thereto (or at least 60%, at least 70%, at least 75%, at least 80%, at least 90%, at least 95%, at least 98% or at least 99% sequence identity thereto).

Reaction (9): SEQ ID NO: 35 or 37 or a sequence having at least 50% sequence identity to either of said sequences (or at least 60%, at least 70%, at least 75%, at least 80%, at least 90%, at least 95%, at least 98% or at least 99% sequence identity thereto).

Reaction (10): SEQ ID NO: 41, 43 or 45 or a sequence having at least 50% sequence identity to any of said sequences (or at least 60%, at least 70%, at least 75%, at least 80%, at least 90%, at least 95%, at least 98% or at least 99% sequence identity thereto).

Reaction (11): SEQ ID NO: 39 or a sequence having at least 50% sequence identity thereto (or at least 60%, at least 70%, at least 75%, at least 80%, at least 90%, at least 95%, at least 98% or at least 99% sequence identity thereto).

Accordingly, a cell according to the invention may express and/or overexpress a polypeptide carrying out the stated reaction. Any polypeptide carrying out the stated reaction may be suitable. Examples include:

Reaction (1): SEQ ID NO: 26 or a sequence having at least 50% sequence identity thereto (or at least 60%, at least 70%, at least 75%, at least 80%, at least 90%, at least 95%, at least 98% or at least 99% sequence identity thereto).

Reaction (2): SEQ ID NO: 24 or a sequence having at least 50% sequence identity thereto (or at least 60%, at least 70%, at least 75%, at least 80%, at least 90%, at least 95%, at least 98% or at least 99% sequence identity thereto).

Reaction (3): SEQ ID NO: 22 or 48 or a sequence having at least 50% sequence identity to either of said sequences (or at least 60%, at least 70%, at least 75%, at least 80%, at least 90%, at least 95%, at least 98% or at least 99% sequence identity thereto).

Reaction (4): SEQ ID NO: 16, 18 or 20 or a sequence having at least 50% sequence identity to any of said sequences (or at least 60%, at least 70%, at least 75%, at least 80%, at least 90%, at least 95%, at least 98% or at least 99% sequence identity thereto).

Reaction (5): SEQ ID NO: 8, 10, 12 or 14 or a sequence having at least 50% sequence identity to any of said sequences (or at least 60%, at least 70%, at least 75%, at least 80%, at least 90%, at least 95%, at least 98% or at least 99% sequence identity thereto).

Reaction (6): SEQ ID NO: 2, 4 or 6 or a sequence having at least 50% sequence identity to any of said sequences (or at least 60%, at least 70%, at least 75%, at least 80%, at least 90%, at least 95%, at least 98% or at least 99% sequence identity thereto).

Reaction (7): SEQ ID NO: 28, 30 or 32 or a sequence having at least 50% sequence identity to any of said sequences (or at least 60%, at least 70%, at least 75%, at least 80%, at least 90%, at least 95%, at least 98% or at least 99% sequence identity thereto).

Reaction (8): SEQ ID NO: 34 or a sequence having at least 50% sequence identity thereto (or at least 60%, at least 70%, at least 75%, at least 80%, at least 90%, at least 95%, at least 98% or at least 99% sequence identity thereto).

Reaction (9): SEQ ID NO: 36 or 38 or a sequence having at least 50% sequence identity to either of said sequences (or at least 60%, at least 70%, at least 75%, at least 80%, at least 90%, at least 95%, at least 98% or at least 99% sequence identity thereto).

Reaction (10): SEQ ID NO: 42, 44 or 46 or a sequence having at least 50% sequence identity to any of said sequences (or at least 60%, at least 70%, at least 75%, at least 80%, at least 90%, at least 95%, at least 98% or at least 99% sequence identity thereto).

Reaction (11): SEQ ID NO: 40 or a sequence having at least 50% sequence identity thereto (or at least 60%, at least 70%, at least 75%, at least 80%, at least 90%, at least 95%, at least 98% or at least 99% sequence identity thereto).

As set out above, combinations of two or more of these reactions may be organized into one or more of the following metabolic pathways including:

PATHWAY 1 comprises at least one or more of the following reaction(s), typically one or more of which are overexpressed:

transportation of cytosolic itaconate to extracellular itaconic acid (eg. SEQ ID NO: 1, 3 or 5 or a sequence having at least 50% sequence identity to any one of said sequences);

conversion of cytosolic cis-aconitate to itaconate (eg. SEQ ID NO: 7, 9, 11 or 13 or a sequence having at least 50% sequence identity to any one of said sequences);

conversion of cytosolic citrate to cis-aconitate (eg. SEQ ID NO: 15, 17 or 19 or a sequence having at least 50% sequence identity to any one of said sequences);

transportation of mitochondrial citrate to the cytosol (eg. SEQ ID NO: 21 or 47 or a sequence having at least 50% sequence identity to any one of said sequences);

conversion of mitochondrial oxaloacetate and acetyl-coenzyme-A into mitochondrial citrate;

transportation of cytosolic oxaloacetate to the mitochondria (eg. SEQ ID NO: 23 or a sequence having at least 50% sequence identity thereto); and

conversion of cytosolic pyruvate and bicarbonate to oxaloacetate (eg. SEQ ID NO: 25 or a sequence having at least 50% sequence identity thereto).

Preferably, in pathway 1, nucleic acids encoding polypeptides having the following activities are overexpressed in a recombinant cell of the invention:
transportation of cytosolic itaconate to extracellular itaconic acid (eg. SEQ ID NO: 1, 3 or 5 or a sequence having at least 50%, at least 60%, at least 70%, at least 75%, at least 80%, at least 90%, at least 95%, at least 98% or at least 99% sequence identity to any one of said sequences);
conversion of cytosolic cis-aconitate to itaconate (eg. SEQ ID NO: 7, 9, 11 or 13 or a sequence having at least 50, at least 60%, at least 70%, at least 75%, at least 80%, at least 90%, at least 95%, at least 98% or at least 99% sequence identity to any one of said sequences);
conversion of cytosolic citrate to cis-aconitate (eg. SEQ ID NO: 15, 17 or 19 or a sequence having at least 50%, at least 60%, at least 70%, at least 75%, at least 80%, at least 90%, at least 95%, at least 98% or at least 99% sequence identity to any one of said sequences);
transportation of mitochondrial citrate to the cytosol (eg. SEQ ID NO: 21 or 47 or a sequence having at least 50%, at least 60%, at least 70%, at least 75%, at least 80%, at least 90%, at least 95%, at least 98% or at least 99% sequence identity to either of said sequences);
transportation of cytosolic oxaloacetate to the mitochondria (eg. SEQ ID NO: 23 or a sequence having at least 50%, at least 60%, at least 70%, at least 75%, at least 80%, at least 90%, at least 95%, at least 98% or at least 99% sequence identity thereto); and
conversion of cytosolic pyruvate and bicarbonate to oxaloacetate (eg. SEQ ID NO: 25 or a sequence having at least 50%, at least 60%, at least 70%, at least 75%, at least 80%, at least 90%, at least 95%, at least 98% or at least 99% sequence identity thereto).

PATHWAY 2 comprises at least one or more of the following reaction(s), typically one or more of which are overexpressed:

transportation of cytosolic itaconate to extracellular itaconic acid (eg. SEQ ID NO: 1, 3 or 5 or a sequence having at least 50% sequence identity to any one of said sequences);

conversion of cytosolic cis-aconitate to itaconate (eg. SEQ ID NO: 7, 9, 11 or 13 or a sequence having at least 50% sequence identity to any one of said sequences);

conversion of cytosolic citrate to cis-aconitate (eg. SEQ ID NO: 15, 17 or 19 or a sequence having at least 50% sequence identity to any one of said sequences;

conversion of cytosolic oxaloacetate and acetyl-coenzyme-A to citrate (eg. SEQ ID NO: 27, 29 or 31 or a sequence having at least 50% sequence identity to any one of said sequences);

conversion of cytosolic acetaldehyde, NAD, and coenzyme-A to acetyl-coenzyme-A and NADH (eg. SEQ ID NO: 33 or a sequence having at least 50% sequence identity thereto);

conversion of cytosolic pyruvate to acetaldehyde and carbon dioxide; and

conversion of cytosolic pyruvate and bicarbonate to oxaloacetate (SEQ ID NO: 25 or a sequence having at least 50% sequence identity thereto).

Preferably, in pathway 2, nucleic acids encoding polypeptides having the following activities are overexpressed in a recombinant cell of the invention:

transportation of cytosolic itaconate to extracellular itaconic acid (eg. SEQ ID NO: 1, 3 or 5 or a sequence having at least 50%, at least 60%, at least 70%, at least 75%, at least 80%, at least 90%, at least 95%, at least 98% or at least 99% sequence identity to any one of said sequences);

conversion of cytosolic cis-aconitate to itaconate (eg. SEQ ID NO: 7, 9, 11 or 13 or a sequence having at least 50%, at least 60%, at least 70%, at least 75%, at least 80%, at least 90%, at least 95%, at least 98% or at least 99% sequence identity to any one of said sequences);

conversion of cytosolic citrate to cis-aconitate (eg. SEQ ID NO: 15, 17 or 19 or a sequence having at least 50%, at least 60%, at least 70%, at least 75%, at least 80%, at least 90%, at least 95%, at least 98% or at least 99% sequence identity to any one of said sequences;

conversion of cytosolic oxaloacetate and acetyl-coenzyme-A to citrate (eg. SEQ ID NO: 27, 29 or 31 or a sequence having at least 50%, at least 60%, at least 70%, at least 75%, at least 80%, at least 90%, at least 95%, at least 98% or at least 99% sequence identity to any one of said sequences);

conversion of cytosolic acetaldehyde, NAD, and coenzyme-A to acetyl-coenzyme-A and NADH (eg. SEQ ID NO: 33 or a sequence having at least 50%, at least 60%, at least 70%, at least 75%, at least 80%, at least 90%, at least 95%, at least 98% or at least 99% sequence identity thereto); and

conversion of cytosolic pyruvate and bicarbonate to oxaloacetate (eg. SEQ ID NO: 25 or a sequence having at least 50%, at least 60%, at least 70%, at least 75%, at least 80%, at least 90%, at least 95%, at least 98% or at least 99% sequence identity thereto).

PATHWAY 3 comprises at least one or more of the following reaction(s), typically one or more of which are overexpressed:

transportation of cytosolic itaconate to extracellular itaconic acid (eg. SEQ ID NO: 1, 3 or 5 or a sequence having at least 50% sequence identity to any one of said sequences);

conversion of cytosolic cis-aconitate to itaconate (eg. SEQ ID NO: 7, 9, 11 or 13 or a sequence having at least 50% sequence identity to any one of said sequences);

conversion of cytosolic citrate to cis-aconitate (eg. SEQ ID NO: 15, 17 or 19 or a sequence having at least 50% sequence identity to any one of said sequences);

conversion of cytosolic oxaloacetate and acetyl-coenzyme-A to citrate (eg. SEQ ID NO: 27, 29 or 31 or a sequence having at least 50% sequence identity to any one of said sequences);

conversion of cytosolic acetyl-phosphate to acetyl-coenzyme-A (eg. SEQ ID NO: 41, 43 or 45 or a sequence having at least 50% sequence identity to any one of said sequences);

conversion of xylulose-5-phosphate and phosphate to acetyl-phosphate and glyceraldehyde 3-phosphate (eg. SEQ ID NO: 35 or 37 or a sequence having at least 50% sequence identity to either of said sequences);

conversion of 6-phosphogluconate and NADP to xylulose-5-phosphate, NADPH and carbon dioxide;

conversion of glucose-6-phosphate and NADP to 6-phosphogluconate and NADPH; and

conversion of cytosolic pyruvate and bicarbonate to oxaloacetate (eg. SEQ ID NO: 25 or a sequence having at least 50% sequence identity thereto).

Preferably, in pathway 3, nucleic acids encoding polypeptides having the following activities are overexpressed in a recombinant cell of the invention:

transportation of cytosolic itaconate to extracellular itaconic acid (eg. SEQ ID NO: 1, 3 or 5 or a sequence having at least 50%, at least 60%, at least 70%, at least 75%, at least 80%, at least 90%, at least 95%, at least 98% or at least 99% sequence identity to any one of said sequences);

conversion of cytosolic cis-aconitate to itaconate (eg. SEQ ID NO: 7, 9, 11 or 13 or a sequence having at least 50%, at least 60%, at least 70%, at least 75%, at least 80%, at least 90%, at least 95%, at least 98%, at least 99% sequence identity to any one of said sequences);

conversion of cytosolic citrate to cis-aconitate (eg. SEQ ID NO: 15, 17 or 19 or a sequence having at least 50%, at least 60%, at least 70%, at least 75%, at least 80%, at least 90%, at least 95%, at least 98% or at least 99% sequence identity to any one of said sequences);

conversion of cytosolic oxaloacetate and acetyl-coenzyme-A to citrate (eg. SEQ ID NO: 27, 29 or 31 or a sequence having at least 50%, at least 60%, at least 70%, at least 75%, at least 80%, at least 90%, at least 95%, at least 98% or at least 99% sequence identity to any one of said sequences);

conversion of cytosolic acetyl-phosphate to acetyl-coenzyme-A (eg. SEQ ID NO: 41, 43 or 45 or a sequence having at least 50%, at least 60%, at least 70%, at least 75%, at least 80%, at least 90%, at least 95%, at least 98% or at least 99% sequence identity to any one of said sequences);

conversion of xylulose-5-phosphate and phosphate to acetyl-phosphate and glyceraldehyde 3-phosphate (eg. SEQ ID NO: 35 or 37 or a sequence having at least 50%, at least 60%, at least 70%, at least 75%, at least 80%, at least 90% or at least 95%, at least 98% or at least 99% sequence identity to either of said sequences); and

conversion of cytosolic pyruvate and bicarbonate to oxaloacetate (eg. SEQ ID NO: 25 or a sequence having at least 50%, at least 60%, at least 70%, at least 75%, at least 80%, at least 90%, at least 95%, at least 98% or at least 99% sequence identity thereto).

PATHWAY 4 comprises at least one or more of the following reaction(s), typically one or more of which are overexpressed:

transportation of cytosolic itaconate to extracellular itaconic acid (eg. SEQ ID NO: 1, 3 or 5 or a sequence having at least 50% sequence identity to any one of said sequences);

conversion of cytosolic cis-aconitate to itaconate (eg. SEQ ID NO: 7, 9, 11 or 13 or a sequence having at least 50% sequence identity to any one of said sequences);

conversion of cytosolic citrate to cis-aconitate (eg. SEQ ID NO: 15, 17 or 19 or a sequence having at least 50% sequence identity to any one of said sequences);

conversion of cytosolic oxaloacetate and acetyl-coenzyme-A to citrate (eg. SEQ ID NO: 27, 29 or 31 or a sequence having at least 50% sequence identity to any one of said sequences);

conversion of cytosolic acetyl-phosphate to acetyl-coenzyme-A (eg. SEQ ID NO: 41, 43 or 45 or a sequence having at least 50% sequence identity to any one of said sequences);

conversion of cytosolic acetate and ATP to acetyl-phosphate, ADP, and phosphate (eg. SEQ ID NO: 39 or a sequence having at least 50% sequence identity thereto);

conversion of cytosolic pyruvate to acetaldehyde and carbon dioxide; and

conversion of cytosolic pyruvate and bicarbonate to oxaloacetate (eg. SEQ ID NO: 25 or a sequence having at least 50% sequence identity thereto).

Preferably, in pathway 4, nucleic acids encoding polypeptides having the following activities are overexpressed in a recombinant cell of the invention:

transportation of cytosolic itaconate to extracellular itaconic acid (eg. SEQ ID NO: 1, 3 or 5 or a sequence having at least 50%, at least 60%, at least 70%, at least 75%, at least 80%, at least 90%, at least 95%, at least 98%, at least 99% sequence identity to any one of said sequences);

conversion of cytosolic cis-aconitate to itaconate (eg. SEQ ID NO: 7, 9, 11 or 13 or a sequence having at least 50%, at least 60%, at least 70%, at least 75%, at least 80%, at least 90%, at least 95%, at least 98% or at least 99% sequence identity to any one of said sequences);

conversion of cytosolic citrate to cis-aconitate (eg. SEQ ID NO: 15, 17 or 19 or a sequence having at least 50%, at least 60%, at least 70%, at least 75%, at least 80%, at least 90%, at least 95%, at least 98% or at least 99% sequence identity to any one of said sequences);

conversion of cytosolic oxaloacetate and acetyl-coenzyme-A to citrate (eg. SEQ ID NO: 27, 29 or 31 or a sequence having at least 50, at least 60%, at least 70%, at least 75%, at least 80%, at least 90%, at least 95%, at least 98% or at least 99% sequence identity to any one of said sequences);

conversion of cytosolic acetyl-phosphate to acetyl-coenzyme-A (eg. SEQ ID NO: 41, 43 or 45 or a sequence having at least 50%, at least 60%, at least 70%, at least 75%, at least 80%, at least 90%, at least 95%, at least 98% or at least 99% sequence identity to any one of said sequences);

conversion of cytosolic acetate and ATP to acetyl-phosphate, ADP, and phosphate (eg. SEQ ID NO: 39 or a sequence having at least 50%, at least 60%, at least 70%, at least 75%, at least 80%, at least 90%, at least 95%, at least 98% or at least 99% sequence identity thereto); and

conversion of cytosolic pyruvate and bicarbonate to oxaloacetate (eg. SEQ ID NO: 25 or a sequence having at least 50%, at least 60%, at least 70%, at least 75%, at least 80%, at least 90%, at least 95%, at least 98% or at least 99% sequence identity thereto).

Each of the pathways described above may be defined in terms of the polypeptides that are overexpressed. Thus, the pathways may be defined in terms of the polypeptides encoded by the nucleic acids defined above (see Tables 4 to 6) and sequences having at least 50%, at least 60%, at least 70%, at least 75%, at least 80%, at least 90%, at least 95%, at least 98% or at least 99% sequence identity to such polypeptides.

In a recombinant cell of the invention, the said cell is been modified in its genome such that it results in a deficiency in the production of a trans-aconitate methyltransferase. The trans-aconitate methyltransferase may be a trans-aconitate 2-methyltransferase (EC2.1.1.144) or a trans-aconitate 3-methyltransferase (EC2.1.1.145)

Deficiency of a recombinant cell in the production of at least one of a trans-aconitate methyltransferase is defined as a phenotypic feature wherein the cell, due to modification in the genome: a) produces less of the polypeptide and/or b) has a reduced expression level of the mRNA transcribed from a gene encoding the polypeptide and/or c) produces a polypeptide having a decreased protein activity or decreased specific protein activity and/or d) produces less of a product produced by the polypeptide and combinations of one or more of these possibilities as compared to a recombinant microorganism that has not been modified in its genome according to the invention, when analysed under substantially identical conditions.

The deficient may be inactivation of a trans-aconitate methyltransferase, for example a knock-out of a gene encoding a trans-aconitate methyltransferase. Thus, there may be no activity of a trans-aconitate methyltransferase in a cell of the invention.

In this context a gene is herewith defined as a polynucleotide containing an open reading frame (ORF) together with its transcriptional control elements (promoter and terminator), the ORF being the region on the gene that will be transcribed and translated into the protein sequence.

Therefore deficiency of a recombinant cell may be measured by determining the amount and/or (specific) activity of the relevant polypeptide produced by the recombinant cell modified in its genome and/or it may be measured by determining the amount of mRNA transcribed from a gene encoding the polypeptide and/or it may be measured by determining the amount of a product produced by the polypeptide in a recombinant microorganism modified in its genome as defined above and/or it may be measured by gene or genome sequencing if compared to the parent host cell which has not been modified in its genome. Deficiency in the production of a polypeptide can be measured using any assay available to the skilled person, such as transcriptional profiling, Northern blotting RT-PCR, Q-PCR and Western blotting.

Modification of a genome of a recombinant cell is herein defined as any event resulting in a change in a polynucleotide sequence in the genome of the cell. A modification is construed as one or more modifications. Modification can be introduced by classical strain improvement, random mutagenesis followed by selection. Modification may be accomplished by the introduction (insertion), substitution or removal (deletion) of one or more nucleotides in a nucleotide sequence. This modification may for example be in a coding sequence or a regulatory element required for the transcription or translation of the polynucleotide. For example, nucleotides may be inserted or removed so as to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32, (7) electronic access http://nar.oupjournals.org/cgi/reprint/32/7/e59 or Gupta et al. (1968), Proc. Natl. Acad. Sci USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of directed mutagenesis procedures are the QuickChange™ site-directed mutagenesis kit (Stratagene Cloning Systems, La Jolla, Calif.), the The Altered Sites® II in vitro Mutagenesis Systems' (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995 Horizon Scientific Press, PO Box 1, Wymondham, Norfolk, U.K.).

A modification in the genome can be determined by comparing the DNA sequence of the modified cell to the sequence of the non-modified cell. Sequencing of DNA and genome sequencing can be done using standard methods known to the person skilled in the art, for example using Sanger sequencing technology and/or next generation sequencing technologies such as Illumina GA2, Roche 454, etc. as reviewed in Elaine R. Mardis (2008), Next-Generation DNA Sequencing Methods, Annual Review of Genomics and Human Genetics, 9: 387-402. (doi:10.1146/annurev.genom.9.081307.164359).

Preferred methods of modification are based on techniques of gene replacement, gene deletion, or gene disruption.

For example, in case of replacement of a polynucleotide, nucleic acid construct or expression cassette, an appropriate DNA sequence may be introduced at the target locus to be replaced. The appropriate DNA sequence is preferably present on a cloning vector. Preferred integrative cloning vectors comprise a DNA fragment, which is homologous to the polynucleotide and/or has homology to the polynucleotides flanking the locus to be replaced for targeting the integration of the cloning vector to this pre-determined locus. In order to promote targeted integration, the cloning vector is preferably linearized prior to transformation of the cell. Preferably, linearization is performed such that at least one but preferably either end of the cloning vector is flanked by sequences homologous to the DNA sequence (or flanking sequences) to be replaced. This process is called homologous recombination and this technique may also be used in order to achieve (partial) gene deletion or gene disruption.

For example, for gene disruption, a polynucleotide corresponding to the endogenous polynucleotide may be replaced by a defective polynucleotide, that is a polynucleotide that fails to produce a (fully functional) protein. By homologous recombination, the defective polynucleotide replaces the endogenous polynucleotide. It may be desirable that the defective polynucleotide also encodes a marker, which may be used for selection of transformants in which the nucleic acid sequence has been modified.

Alternatively, modification, wherein said host cell produces less of or is deficient in the production of one of the polypeptides described herein may be performed by established anti-sense techniques using a nucleotide sequence complementary to the nucleic acid sequence of the polynucleotide. More specifically, expression of the polynucleotide by a host cell may be reduced or eliminated by introducing a nucleotide sequence complementary to the nucleic acid sequence of the polynucleotide, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl. Environ. Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52.).

Furthermore, modification, downregulation or inactivation of a polynucleotide may be obtained via the RNA interference (RNAi) technique (FEMS Microb. Lett. 237 (2004): 317-324). In this method identical sense and antisense parts of the nucleotide sequence, which expression is to be affected, are cloned behind each other with a nucleotide spacer in between, and inserted into an expression vector. After such a molecule is transcribed, formation of small nucleotide fragments will lead to a targeted degradation of the mRNA, which is to be affected. The elimination of the specific mRNA can be to various extents. The RNA interference techniques described in WO2008/053019, WO2005/05672A1, WO2005/026356A1, Oliveira et al., “Efficient cloning system for construction of gene silencing vectors in Aspergillus niger” (2008) Appl. Microbiol. and Biotechnol. 80 (5): 917-924 and/or Barnes et al., “siRNA as a molecular tool for use in Aspergillus niger” (2008) Biotechnology Letters 30 (5): 885-890 may be used for downregulation, modification or inactivation of a polynucleotide.

Preferably, in a recombinant cell according to the invention, the deficiency in the production of one or more of the polypeptides identified herein is a reduction in production of at least 20% more preferably by at least 30%, more preferably by at least 40%, even more preferably at least 50%, even more preferably at least 60%, in particular at least 70%, more in particular at least 80%, for example at least 85%, for example at least 90%, for example at least 95%, for example at least 100% (as compared to a recombinant microorganism that has not been modified in its genome according to the invention, when analysed under substantially identical conditions).

Preferably, the modification in the genome of the recombinant cell according to the invention is a modification in the genome on at least one position of at least one nucleic acid sequence encoding a polypeptide having at least 30% identity, such as 35% identity, more preferably at least 40% identity, more preferably at least 45% identity, more preferably at least 50% identity, even more preferably at least 55% identity, even more preferably at least 60% identity, even more preferably at least 65% identity, even more preferably at least 70% identity, even more preferably at least 75% identity, even more preferably at least 80% identity, even more preferably at least 85% identity, even more preferably at least 90% identity, for example at least 91% identity, for example at least 92% identity, for example at least 93% identity, for example at least 94% identity, for example at least 95% identity, for example at least 96% identity, for example at least 97% identity, for example at least 98% identity, for example at least 99% identity, for example 100% identity with a polypeptide selected from a polypeptide according to SEQ ID NO: 65 and SEQ ID NO: 66 and/or the modification in the genome of the microbial host cell in the method according to the invention is a modification resulting in the reduction of the amount of at least one mRNA having at least 30% identity, at least 60% identity, even more preferably at least 65% identity, even more preferably at least 70% identity, even more preferably at least 75% identity, even more preferably at least 80% identity, even more preferably at least 85% identity, even more preferably at least 90% identity, for example at least 91% identity, for example at least 92% identity, for example at least 93% identity, for example at least 94% identity, for example at least 95% identity, for example at least 96% identity, for example at least 97% identity, for example at least 98% identity, for example at least 99% identity, for example 100% identity with an mRNA selected from the group of the mRNA according to SEQ ID NO: 68 or SEQ ID NO: 69.

In each case, the modification typically takes place in an mRNA sequence or a nucleic acid sequence encoding polypeptide encoding or having the same activity as the given SEQ ID NO.

According to the invention, there is thus provided a genetically modified yeast comprising one or more of these metabolic pathways, whereby overexpression of one or more enzymes on these metabolic pathways confers yeast cell the ability to produce elevated levels of itaconic acid.

Also, provided is a cell which is capable of producing one or more of 4-methyl itaconate or 1-methyl itaconate. Typically, such a recombinant cell is one in which one or more nucleic acid sequences encoding a polypeptide are overexpressed, said polypeptide(s) being capable of catalyzing one or more of the conversions:
a. cis-aconitate to itaconate (eg. SEQ ID NOs: 7, 9, 11 or 13 or a sequence having at least 50%, at least 60%, at least 70%, at least 75%, at least 80%, at least 90%, at least 95%, at least 98% or at least 99% sequence identity to any of said sequences);
b. itaconate to 4-methyl itaconate (eg. SEQ ID NO: 69 or a sequence having at least 50%, at least 60%, at least 70%, at least 75%, at least 80%, at least 90%, at least 95%, at least 98% or at least 99% sequence identity thereto);
c. itaconate to 1-methyl itaconate (eg. SEQ ID NO: 68 or a sequence having at least 50%, at least 60%, at least 70%, at least 75%, at least 80%, at least 90%, at least 95%, at least 98% or at least 99% sequence identity thereto);
d. cis-aconitate to trans-aconitate (eg. SEQ ID NO: 70 or a sequence having at least 50%, at least 60%, at least 70%, at least 75%, at least 80%, at least 90%, at least 95%, at least 98% or at least 99% sequence identity thereto);
e. trans-aconitate to (E)-3-carboxy-2-pentenedioate 5-methyl ester (eg. SEQ ID NO: 69 or a sequence having at least 50%, at least 60%, at least 70%, at least 75%, at least 80%, at least 90%, at least 95%, at least 98% or at least 99% sequence identity thereto);
f. trans-aconitate to (E)-3-(methoxycarbonyl)pent-2-enedioate (eg. SEQ ID NO: 68 or a sequence having at least 50%, at least 60%, at least 70%, at least 75%, at least 80%, at least 90%, at least 95%, at least 98% or at least 99% sequence identity thereto);
g. (E)-3-carboxy-2-pentenedioate 5-methyl ester to 4-methyl itaconate (eg. SEQ ID NOs: 7, 9, 11 or 13 or a sequence having at least 50%, at least 60%, at least 70%, at least 75%, at least 80%, at least 90%, at least 95%, at least 98% or at least 99% sequence identity to any of said sequences); and
h. (E)-3-(methoxycarbonyl)pent-2-enedioate to 1-methyl itaconate (eg. SEQ ID NOs: 7, 9, 11 or 13 or a sequence having at least 50%, at least 60%, at least 70%, at least 75%, at least 80%, at least 90%, at least 95%, at least 98% or at least 99% sequence identity to any of said sequences).

Typically, such a recombinant cell is one in which one or more polypeptides are overexpressed, said polypeptide(s) being capable of catalyzing one or more of the conversions:
a. cis-aconitate to itaconate (eg. SEQ ID NOs: 8, 10, 12 or 14 or a sequence having at least 50%, at least 60%, at least 70%, at least 75%, at least 80%, at least 90%, at least 95%, at least 98% or at least 99% sequence identity to any of said sequences);
b. itaconate to 4-methyl itaconate (eg. SEQ ID NO: 66 or a sequence having at least 50%, at least 60%, at least 70%, at least 75%, at least 80%, at least 90%, at least 95%, at least 98% or at least 99% sequence identity thereto);
c. itaconate to 1-methyl itaconate (eg. SEQ ID NO: 65 or a sequence having at least 50%, at least 60%, at least 70%, at least 75%, at least 80%, at least 90%, at least 95%, at least 98% or at least 99% sequence identity thereto);
d. cis-aconitate to trans-aconitate (eg. SEQ ID NO: 67 or a sequence having at least 50%, at least 60%, at least 70%, at least 75%, at least 80%, at least 90%, at least 95%, at least 98% or at least 99% sequence identity thereto);
e. trans-aconitate to (E)-3-carboxy-2-pentenedioate 5-methyl ester (eg. SEQ ID NO: 66 or a sequence having at least 50%, at least 60%, at least 70%, at least 75%, at least 80%, at least 90%, at least 95%, at least 98% or at least 99% sequence identity thereto);
f. trans-aconitate to (E)-3-(methoxycarbonyl)pent-2-enedioate (eg. SEQ ID NO: 65 or a sequence having at least 50%, at least 60%, at least 70%, at least 75%, at least 80%, at least 90%, at least 95%, at least 98% or at least 99% sequence identity thereto);
g. (E)-3-carboxy-2-pentenedioate 5-methyl ester to 4-methyl itaconate (eg. SEQ ID NOs: 8, 10, 12 or 14 or a sequence having at least 50%, at least 60%, at least 70%, at least 75%, at least 80%, at least 90%, at least 95%, at least 98% or at least 99% sequence identity to any of said sequences); and
h. (E)-3-(methoxycarbonyl)pent-2-enedioate to 1-methyl itaconate (eg. SEQ ID NOs: 8, 10, 12 or 14 or a sequence having at least 50%, at least 60%, at least 70%, at least 75%, at least 80%, at least 90%, at least 95%, at least 98% or at least 99% sequence identity to any of said sequences).

A recombinant cell of the invention which is capable of producing 1-methyl itaconate may comprise one or more nucleic acid sequences encoding polypeptides capable of catalyzing the conversions:
a and c; or
d, f and h.

Such a recombinant cell may may be defined in terms of the polypeptides that are overexpressed. Thus, the pathways may be defined in terms of the polypeptides encoded by the nucleic acids defined above (see Tables 4 to 6) and sequences having at least 50%, at least 60%, at least 70%, at least 75%, at least 80%, at least 90%, at least 95%, at least 98% or at least 99% sequence identical to such polypeptides.

A recombinant cell of the invention which is capable of producing 4-methyl itaconate may comprise one or more nucleic acid sequences encoding polypeptides capable of catalyzing the conversions:
a and b; or
d, e, and g.

Such a recombinant cell may may be defined in terms of the polypeptides that are overexpressed. Thus, the pathways may be defined in terms of the polypeptides encoded by the nucleic acids defined above (see Tables 4 to 6) and sequences having at least 50%, at least 60%, at least 70%, at least 75%, at least 80%, at least 90%, at least 95%, at least 98% or at least 99% sequence identity to such polypeptides.

The conversions identified above are defined with reference to specific nucleic acids or polypeptides. These nucleic acids and polypeptides are given merely be way of example and should not be seen as limiting. Any suitable nucleic acid can be used which encodes a polypeptide having the desired activity or any polypeptide having the desired activity may be used. Sequences related to those specifically set out herein may be used in the invention.

A suitable nucleic acid may encode a polypeptide as encoded by one of the nucleic acids identified above or a polypeptide shared at least about 50%, at least about 60%, at least about 70%, at least about 80%, at least about 90%, at least about 95%, at least about 98%, at least about 99% sequence identity with a polypeptide encoded by one of the nucleic acids identified herein.

That is to say, nucleic acids and polypeptides suitable for use in the herein may be have at least 50%, at least 55% at least 60%, at least 65% at least 70%, at least 75%, at least 80%, at least 85% at least 90%, at least 95%, at least 97%, at least 98%, at least 99% sequence identity with a nucleic acid or polypeptide specifically identified herein.

According to the invention, there is thus further provided that metabolic pathways comprising reactions catalyzed by the amino acid sequences listed in Table 4, whereby overexpression of one or more of those amino acid sequences within the same metabolic pathway in a genetically modified yeast cell confers yeast cell the ability to produce elevated levels of itaconic acid or ester of itaconic acid.

Expression levels of these amino acid sequences in a recombinant cell may be controlled by constitutive strong promoters conferring on a recombinant cell the ability to produce elevated levels of itaconic acid and/or an ester of itaconic.

According to the invention, there is thus further provided that a genetically modified yeast cell comprising one or more overexpression of the metabolic pathways as mentioned above and deletion of pyruvate decarboxylase, alcohol dehydrogenase, isocitrate dehydrogenase, alpha-ketoglutarate dehydrogenase, or succinyl-CoA ligase whereby the deletion confers yeast cell the ability to produce elevated levels of itaconic acid and itaconate methyl ester.

As used herein, a recombinant cell or recombinant yeast cell according to the present invention is defined as a cell which contains, or is transformed or genetically modified with one or more nucleotide sequence and/or protein that does not naturally occur in the yeast, or it contains additional copy or copies of an endogenous nucleic acid sequence (or protein). A wild-type cell or yeast cell is herein defined as the parental cell or yeast cell of the recombinant cell or yeast cell.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but have been obtained from another cell or synthetically or recombinantly produced.

Sequence identity is herein defined as a relationship between two or more amino acid (polypeptide or protein) sequences or two or more nucleic acid (polynucleotide) sequences, as determined by comparing the sequences. Usually, sequences are compared over the whole length of the sequences compared. In the art, “identity” also means the degree of sequence relatedness between amino acid or nucleic acid sequences, as the case may be, as determined by the match between strings of such sequences.

The parameter “identity” as used herein describes the relatedness between two amino acid sequences or between two nucleotide sequences. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

A nucleotide sequence encoding an enzyme which catalyses a conversion as set out herein may also be defined by its capability to hybridise with the nucleotide sequences encoding an enzyme capable catalyzing the reaction, under moderate, or preferably under stringent hybridisation conditions.

Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC (sodium chloride, sodium citrate) or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequence of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

The term “gene”, as used herein, refers to a nucleic acid sequence containing a template for a nucleic acid polymerase, in eukaryotes, RNA polymerase II. Genes are transcribed into mRNAs that are then translated into protein.

The term “nucleic acid” as used herein, includes reference to a deoxyribonucleotide or ribonucleotide polymer, i.e. a polynucleotide, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). A polynucleotide can be full-length or a subsequence of a native or heterologous structural or regulatory gene. Unless otherwise indicated, the term includes reference to the specified sequence as well as the complementary sequence thereof.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

The term “enzyme” as used herein is defined as a protein which catalyses a (bio)chemical reaction in a cell, such as a yeast cell.

To increase the likelihood that the introduced enzyme is expressed in active form in a yeast of the invention, the corresponding encoding nucleotide sequence may be adapted to optimise its codon usage to that of the chosen yeast cell. Several methods for codon optimisation are known in the art. A preferred method to optimise codon usage of the nucleotide sequences to that of the yeast is a codon pair optimization technology as disclosed in WO2008/000632. Codon-pair optimization is a method for producing a polypeptide in a host cell, wherein the nucleotide sequences encoding the polypeptide have been modified with respect to their codon-usage, in particular the codon-pairs that are used, to obtain improved expression of the nucleotide sequence encoding the polypeptide and/or improved production of the polypeptide. Codon pairs are defined as a set of two subsequent triplets (codons) in a coding sequence.

Usually, the nucleotide sequence encoding an enzyme introduced into a cell of the invention is operably linked to a promoter that causes sufficient expression of the corresponding nucleotide sequence in the cell according to the present invention to confer on the cell the ability to the enzyme.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences known to a person skilled in the art. A “constitutive” promoter is a promoter that is active under most environmental and developmental conditions. An “inducible” promoter is a promoter that is active under environmental or developmental regulation.

A promoter that could be used to achieve the expression of a nucleotide sequence coding for an enzyme may be not native to the nucleotide sequence coding for the enzyme to be expressed, i.e. a promoter that is heterologous to the nucleotide sequence (coding sequence) to which it is operably linked. Preferably, the promoter is homologous, i.e. endogenous to the host cell.

Suitable promoters in this context include both constitutive and inducible natural promoters as well as engineered promoters, which are well known to the person skilled in the art. Suitable promoters in eukaryotic host cells may be GAL7, GAL10, or GAL 1, CYC1, HIS3, ADH1, PGL, PH05, GAPDH, ADC1, TRP1, URA3, LEU2, ENO, TPI, and AOX1. Other suitable promoters include PDC, GPD1, PGK1, TEF1, and TDH.

Usually a nucleotide sequence encoding an enzyme comprises a terminator. Any terminator, which is functional in the cell, may be used in the present invention. Preferred terminators are obtained from natural genes of the host cell. Suitable terminator sequences are well known in the art. Preferably, such terminators are combined with mutations that prevent nonsense mediated mRNA decay in the host cell of the invention (see for example: Shirley et al., 2002, Genetics 161:1465-1482).

In the invention, the nucleotide sequence encoding an enzyme that catalyses a conversion as described herein may be overexpressed to achieve increased production of that enzyme in a recombinant cell according to the present invention.

There are various means available in the art for overexpression of nucleotide sequences encoding enzymes in the yeast cell of the invention. In particular, a nucleotide sequence encoding an enzyme may be overexpressed by increasing the copy number of the gene coding for the enzyme in the cell, e.g. by integrating additional copies of the gene in the cell's genome, by expressing the gene from a centromeric vector, from an episomal multicopy expression vector or by introducing an (episomal) expression vector that comprises multiple copies of the gene. Preferably, overexpression of the enzyme according to the invention is achieved with a (strong) constitutive promoter.

The nucleic acid construct may be a plasmid, for instance a low copy plasmid or a high copy plasmid. The yeast according to the present invention may comprise a single or multiple copies of a nucleotide sequence encoding an enzyme encoding a given conversion, for instance by multiple copies of a nucleotide construct.

The nucleic acid construct may be maintained episomally and thus comprise a sequence for autonomous replication, such as an autosomal replication sequence sequence. A suitable episomal nucleic acid construct may e.g. be based on the yeast 2p or pKD1 plasmids (Gleer et al., 1991, Biotechnology 9: 968-975), or the AMA plasmids (Fierro et al., 1995, Curr Genet. 29:482-489). Alternatively, each nucleic acid construct may be integrated in one or more copies into the genome of the yeast cell. Integration into the cell's genome may occur at random by non-homologous recombination but preferably, the nucleic acid construct may be integrated into the cell's genome by homologous recombination as is well known in the art (see e.g. WO90/14423, EP-A-0481008, EP-A-0635 574 and U.S. Pat. No. 6,265,186).

With the exception of transporter polypeptides, in the invention, it is preferred the enzyme or enzymes expressed in a recombinant cell of the invention is/are active in the cytosol upon expression of the encoding nucleotide sequence(s). Cytosolic activity of the enzyme(s) is/are preferred for a high productivity of itaconic acid or an itaconic acid ester by the cell.

A nucleotide sequence encoding an enzyme that catalyses a conversion as described herein, may comprise a peroxisomal or mitochondrial targeting signal, for instance as determined by the method disclosed by Schluter et al, Nucleic acid Research 2007, Vol 25, D815-D822. In the event the enzyme comprises a targeting signal, it may be preferred that the yeast according to the invention comprises a truncated form of the enzyme, wherein the targeting signal is removed.

A recombinant cell of the invention may be a yeast cell. The yeast according to the present invention preferably belongs to one of the genera Saccharomyces, Pichia, Kluyveromyces, or Zygosaccharomyces. More preferably, the yeast cell may be Saccharomyces cerevisiae, Saccharomyces uvarum, Saccharomyces bayanus, Pichia stipidis, Kluyveromyces marxianus, K. lactis, K. thermotolerans, or Zygosaccharomyces bailii.

In a preferred embodiment, the yeast according to the present invention may be able to grow on any suitable carbon source known in the art and convert it to itaconic acid or an itaconic acid ester. The yeast may be able to convert directly plant biomass, celluloses, hemicelluloses, pectines, rhamnose, galactose, fructose, maltose, maltodextrines, ribose, ribulose, or starch, starch derivatives, sucrose, lactose and glycerol. Hence, a preferred yeast cell expresses enzymes such as cellulases (endocellulases and exocellulases) and hemicellulases (e.g. endo- and exo-xylanases, arabinases) necessary for the conversion of cellulose into glucose monomers and hemicellulose into xylose and arabinose monomers, pectinases able to convert pectines into glucuronic acid and galacturonic acid or amylases to convert starch into glucose monomers. The ability of a yeast to express such enzymes may be naturally present or may have been obtained by genetic modification of the yeast. Preferably, the yeast is able to convert a carbon source selected from the group consisting of glucose, fructose, galactose, xylose, arabinose, sucrose, lactose, raffinose and glycerol.

In another aspect, the present invention relates to a process for the preparation of itaconic acid or an itaconic acid ester, which process comprises fermenting a yeast cell according to the present invention in the presence of a suitable fermentation medium. Suitable fermentation media are known to the skilled man in the art. Preferably, the itaconic acid ester produced in the process according to the present invention is 4-methyl itaconate or 1-methyl itaconate.

The process for the production of itaconic acid or an itaconic acid ester according to the present invention may be carried out at any suitable pH between 1 and 9. Preferably, the pH in the fermentation broth is between 2 and 7, preferably between 3 and 5. It was found advantageous to be able to carry out the process according to the present invention at a low pH, since this prevents bacterial contamination. In addition, since the pH drops during itaconic acid production, a lower amount of titrant is needed to keep the pH at a desired level.

A suitable temperature at which the process according to the present invention may be carried out is between 5 and 60° C., preferably between 10 and 50° C., more preferably between 15 and 35° C., more preferably between 18° C. and 30° C. The skilled man in the art knows which optimal temperatures are suitable for fermenting a specific yeast cell.

Preferably, the itaconic acid or itaconic acid ester is recovered from the fermentation broth by a suitable method known in the art, for instance by crystallisation.

Preferably, the itaconic acid or an ester of itaconic acid that is prepared in the process according to the present invention is further converted into a desirable product, such as a pharmaceutical, cosmetic, food, feed or chemical product. In particular, itaconic acid or an ester of itaconic acid may be further converted into a polymer.

Standard genetic techniques, such as overexpression of enzymes in the host cells, genetic modification of host cells, or hybridisation techniques, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation, genetic modification etc of fungal host cells are known from e.g. EP-A-0 635 574, WO 98/46772, WO 99/60102 and WO 00/37671, WO90/14423, EP-A-0481008, EP-A-0635 574 and U.S. Pat. No. 6,265,186.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

### Embodiments of the Invention

1. A recombinant cell which is capable of producing one or more of itaconic acid, 4-methyl itaconate or 1-methyl itaconate, wherein the said recombinant cell has been modified in its genome such that it results in a deficiency in the production of a trans-aconitate methyltransferase.
2. A recombinant cell according to embodiment 1 in which one or more nucleic acid sequences encoding a polypeptide are overexpressed, said polypeptide(s) being capable of catalyzing one or more of the conversions:
   a. cis-aconitate to itaconate;
   b. itaconate to 4-methyl itaconate;
   c. itaconate to 1-methyl itaconate;
   d. cis-aconitate to trans-aconitate;
   e. trans-aconitate to (E)-3-carboxy-2-pentenedioate 5-methyl ester;
   f. trans-aconitate to (E)-3-(methoxycarbonyl)pent-2-enedioate;
   g. (E)-3-carboxy-2-pentenedioate 5-methyl ester to 4-methyl itaconate; and
   h. (E)-3-(methoxycarbonyl)pent-2-enedioate to 1-methyl itaconate.
3. A recombinant cell according to embodiment 2 which is capable of producing 1-methyl itaconate and which comprises one or more nucleic acid sequences encoding polypeptides capable of catalyzing the conversions:
   a and c; or
   d, f and h.
4. A recombinant cell according to embodiment 2 or 3 which is capable of producing 4-methyl itaconate and which comprises one or more nucleic acid sequences encoding polypeptides capable of catalyzing the conversions:
   a and b; or
   d, e, and g.
5. A recombinant cell according to any one of the preceding embodiments which is a yeast cell.
6. A recombinant yeast cell, optionally according to any one of the preceding embodiments, which is capable of producing itaconic acid and which overexpresses:
   a nucleic acid encoding a polypeptide having cis-aconitate decarboxylase activity; and
   one or more nucleic acids encoding polypeptides which separately or together catalyze a reaction towards acetyl CoA,
   wherein the said recombinant microorganism has been modified in its genome such that it results in a deficiency in the production of a trans-aconitate methyltransferase
7. A recombinant yeast cell according to embodiment 6, wherein the nucleic acid encoding a polypeptide which catalyzes a reaction towards acetyl CoA is
   nucleic acid sequences encoding polypeptides which together have pyruvate dehydrogenase activity;
   one or more nucleic acid sequences encoding one or more polypeptides having pyruvate decarboxylase activity, acetaldehyde dehydrogenase activity and/or acetyl-CoA synthetase activity;
   a nucleic acid sequence encoding a polypeptide having acetylating acetaldehyde dehydrogenase activity;
   a nucleic acid sequence encoding a polypeptide having pyruvate:NADP oxidoreductase activity;
   a nucleic acid encoding a polypeptide having acetate:CoA ligase (ADP-forming) activity;
   a nucleic acid encoding a polypeptide ATP:acetate phosphotransferase activity and a nucleic acid encoding a polypeptide having acetyl-CoA:Pi acetyltransferase activity/phosphate acetyltransferase activity.
8. A recombinant cell according to any one of the preceding embodiments which overexpresses:
   a nucleic acid encoding a polypeptide catalyzing conversion of citrate to cis-aconitate; and/or
   a nucleic acid encoding a polypeptide having citrate synthase activity.
9. A recombinant cell according to any one of the preceding embodiments which overexpresses:
   a nucleic acid encoding a polypeptide having pyruvate carboxylase; and/or
   a nucleic acid encoding a polypeptide having PEP carboxykinase activity; and/or
   a nucleic acid encoding a polypeptide having PEP carboxylase.
10. A recombinant cell according to any one of the preceding embodiments which overexpresses:
   a nucleic acid sequence encoding a mitochondrial membrane citrate transporter.
11. A recombinant cell according to any one of the preceding embodiments which comprises:
   a nucleic acid sequence encoding a itaconic acid transporter, a 4-methyl itaconate transporter or a 1-methyl itaconate transporter.
12. A recombinant cell according to any one of the preceding embodiments comprising a genetic modification resulting in reduced expression and/or activity of pyruvate decarboxylase, alcohol dehydrogenase, isocitrate dehydrogenase, alpha-ketoglutarate dehydrogenase, or succinyl-CoA ligase in the cell as compared to a cell without the genetic modification.
13. A recombinant cell according to any one of the previous embodiments which is a yeast cell, such as a S. cerevisiae cell.
14. A recombinant cell, optionally according to any one of embodiments 1 to 13, which comprises, for example overexpresses, polypeptides catalysing the following reactions:
   transportation of cytosolic itaconate to extracellular itaconic acid;
   conversion of cytosolic cis-aconitate to itaconate;
   conversion of cytosolic citrate to cis-aconitate;
   conversion of cytosolic oxaloacetate and acetyl-coenzyme-A to citrate;
   conversion of cytosolic acetaldehyde, NAD, and coenzyme-A to acetyl-coenzyme-A and NADH; and
   conversion of cytosolic pyruvate and bicarbonate to oxaloacetate.
15. A recombinant cell, optionally according to any one of embodiments 1 to 13, which comprises, for example overexpresses, polypeptides catalysing the following reactions:
   transportation of cytosolic itaconate to extracellular itaconic acid;
   conversion of cytosolic cis-aconitate to itaconate;
   conversion of cytosolic citrate to cis-aconitate;
   transportation of mitochondrial citrate to the cytosol;
   transportation of cytosolic oxaloacetate to the mitochondria; and
   conversion of cytosolic pyruvate and bicarbonate to oxaloacetate.
16. A recombinant cell, optionally according to any one of embodiments 1 to 13, which comprises, for example overexpresses, polypeptides catalysing the following reactions:
   transportation of cytosolic itaconate to extracellular itaconic acid;
   conversion of cytosolic cis-aconitate to itaconate;
   conversion of cytosolic citrate to cis-aconitate;
   conversion of cytosolic oxaloacetate and acetyl-coenzyme-A to citrate;
   conversion of cytosolic acetyl-phosphate to acetyl-coenzyme-A;
   conversion of xylulose-5-phosphate and phosphate to acetyl-phosphate and glyceraldehyde 3-phosphate; and
   conversion of cytosolic pyruvate and bicarbonate to oxaloacetate.
17. A recombinant cell, optionally according to any one of embodiments 1 to 13, which comprises, for example overexpresses, polypeptides catalysing the following reactions:
   transportation of cytosolic itaconate to extracellular itaconic acid;
   conversion of cytosolic cis-aconitate to itaconate;
   conversion of cytosolic citrate to cis-aconitate;
   conversion of cytosolic oxaloacetate and acetyl-coenzyme-A to citrate;
   conversion of cytosolic acetyl-phosphate to acetyl-coenzyme-A;
   conversion of cytosolic acetate and ATP to acetyl-phosphate, ADP, and phosphate; and
   conversion of cytosolic pyruvate and bicarbonate to oxaloacetate.
18. A recombinant cell according to any one of embodiments 14 to 17 which is a yeast cell, such as a Saccharomyces cerevisiae cell.
19. A recombinant cell according to any one of the preceding embodiments wherein said recombinant microorganism has been modified in its genome such that it results in a deficiency in the production of a trans-aconitate methyltransferase comprising an amino acid sequence having at least about 30% sequence identity with SEQ ID NO: 66.
20. A process for the production of 4-methyl itaconate or 1-methyl itaconate, which process comprises fermenting a recombinant cell according to any one of embodiments 1 to 5 or 8 to 19 in a suitable fermentation medium, wherein 4-methyl itaconate or 1-methyl itaconate is produced.
21. A process for the production of itaconic acid or an ester of itaconic acid, which process comprises fermenting a recombinant cell according to any one of embodiments 6 to 19 in a suitable fermentation medium, wherein the itaconic acid or ester of itaconic acid is produced.
22. A process according to embodiments 20 or 21, wherein the itaconic acid or ester of itaconic acid is further converted into a pharmaceutical, cosmetic, food, feed or chemical product.
23. A fermentation broth comprising a itaconic acid and/or an ester of itaconate obtainable by a process according to claim any one of embodiments 20 to 22.

The present invention is further illustrated by the following Examples:

### EXAMPLES

### Example 1: Overexpression of Enzymes for Different Metabolic Pathways for Itaconic Acid and Itaconate Methyl Ester Production in Saccharomyces cerevisiae

1.1 Expression Constructs

The nucleotide sequences of SEQ ID NOs 1, 3, 5, 7, 9, 11, 13, 15, 17, 19, 21, 23, 25, 27, 29, 31, 33, 35, 37, 39, 41, 43, 45, and 47 are obtained by the codon-pair optimization method as disclosed in PCT/EP2007/05594 for S. cerevisiae were synthesized. The nucleotide sequences of SEQ ID NOs 49, 50, 51, 52, 53, 54, 55, 56, 57, 58, 59, 60, 61, 62, 63 and 64 were synthesized. From these sequences (promoter, open reading frame and terminators) expression cassettes were built according to the methods described in the co-pending patent application no. U.S. 61/616,254 and WO2013/144257. The formed expression cassettes (cassette 117-cassette 149) were used as a template to PCR amplify the DNA fragments used in the transformation.

1.2 Preparation and Purification of PCR Fragments for Transformation

Assembly and integration of the itaconic acid pathways is done according to the described methods in the co-pending patent application no. U.S. 61/616,254 and WO2013/144257. Amplification of expression cassettes with connector sequences from the plasmids was carried out with a standard set of primers binding to the connectors. The primers are set out in SEQ ID NOs: 87 to 110 of the co-pending patent application no. U.S. 61/616,254 and WO2013/144257 and named after the connector and the direction of amplification. For example “con 5 fw” was the forward primer on connector 5. Only a subset of the primers was used in this experiment. Table 1 shows the primers used with the corresponding PCR templates used in the PCR reactions. PCR reactions were performed with Phusion polymerase (Finnzymes) according to the manual.

TABLE 1
Overview of all cassettes, the content of the cassettes and the primer
combinations for generating expression cassettes equipped with connectors used in the
transformation of S. cerevisiae

| cassette Nos | forward | reverse | PRO | ORF | TER | BBN |
|---|---|---|---|---|---|---|
| CAS117 | con5 forw | conA rev | Sc Act1.pro | SEQ ID NO: 1 | ADH1 terminator | Sc 5a.bbn |
| CAS118 | | | Sc Act1.pro | SEQ ID NO: 3 | ADH1 terminator | Sc 5a.bbn |
| CAS119 | | | Sc Act1.pro | SEQ ID NO: 5 | ADH1 terminator | Sc 5a.bbn |
| CAS120 | conB forw | conC rev | Sc TDH3.pro | SEQ ID NO: 7 | TDH1 terminator | Sc bc.bbn |
| CAS121 | | | Sc TDH3.pro | SEQ ID NO: 9 | TDH1 terminator | Sc bc.bbn |
| CAS122 | | | Sc TDH3.pro | SEQ ID NO: 11 | TDH1 terminator | Sc bc.bbn |
| CAS123 | | | Sc TDH3.pro | SEQ ID NO: 13 | TDH1 terminator | Sc bc.bbn |
| CAS133 | conC forw | conD rev | Sc FBA1.pro | SEQ ID NO: 15 | GPM1 terminator | Sc cd.bbn |
| CAS134 | | | Sc FBA1.pro | SEQ ID NO: 17 | GPM1 terminator | Sc cd.bbn |
| CAS135 | | | Sc FBA1.pro | SEQ ID NO: 19 | GPM1 terminator | Sc cd.bbn |
| CAS144 | | | Sc PRE3.pro | SEQ ID NO: 15 | GPM1 terminator | Sc cd.bbn |
| CAS145 | | | Sc PRE3.pro | SEQ ID NO: 17 | GPM1 terminator | Sc cd.bbn |
| CAS146 | | | Sc PRE3.pro | SEQ ID NO: 19 | GPM1 terminator | Sc cd.bbn |
| CAS136 | con D forw | con E rev | Sc PGK1.pro | SEQ ID NO: 25 | TPI1 terminator | Sc de.bbn |
| CAS124 | conE forw | conF rev | Sc Tef1.pro | SEQ ID NO: 21 | PDC1 terminator | Sc ef.bbn |
| CAS125 | | | Sc Tef1.pro | SEQ ID NO: 47 | PDC1 terminator | Sc ef.bbn |
| CAS137 | | | Sc Tef1.pro | SEQ ID NO: 27 | PDC1 terminator | Sc ef.bbn |
| CAS138 | | | Sc Tef1.pro | SEQ ID NO: 29 | PDC1 terminator | Sc ef.bbn |
| CAS139 | | | Sc Tef1.pro | SEQ ID NO: 31 | PDC1 terminator | Sc ef.bbn |
| CAS147 | | | Sc TDH1.pro | SEQ ID NO: 27 | PDC1 terminator | Sc ef.bbn |
| CAS148 | | | Sc TDH1.pro | SEQ ID NO: 29 | PDC1 terminator | Sc ef.bbn |
| CAS149 | | | Sc TDH1.pro | SEQ ID NO: 31 | PDC1 terminator | Sc ef.bbn |
| CAS126 | conF forw | con3 rev | Sc ENO2.pro | SEQ ID NO: 23 | TAL1 terminator | Sc f3.bbn |
| CAS130 | | | Sc ENO2.pro | SEQ ID NO: 41 | TAL1 terminator | Sc f3.bbn |
| CAS131 | | | Sc ENO2.pro | SEQ ID NO: 43 | TAL1 terminator | Sc f3.bbn |
| CAS132 | | | Sc ENO2.pro | SEQ ID NO: 45 | TAL1 terminator | Sc f3.bbn |
| CAS140 | | | Sc ENO2.pro | SEQ ID NO: 33 | TAL1 terminator | Sc f3.bbn |
| CAS141 | FG | FG | Sc ENO2.pro | SEQ ID NO: 41 | TAL1 terminator | Sc fg.bbn |
| CAS142 | | | Sc ENO2.pro | SEQ ID NO: 43 | TAL1 terminator | Sc fg.bbn |
| CAS143 | | | Sc ENO2.pro | SEQ ID NO: 45 | TAL1 terminator | Sc fg.bbn |
| CAS127 | G3 | G4 | Sc PGI1.pro | SEQ ID NO: 35 | TDH3 terminator | Sc g3.bbn |
| CAS128 | | | Sc PGI1.pro | SEQ ID NO: 37 | TDH3 terminator | Sc g3.bbn |
| CAS129 | | | Sc PGI1.pro | SEQ ID NO: 39 | TDH3 terminator | Sc g3.bbn |

The dominant marker KanMX is amplified using a standard plasmid containing the fragments as template DNA. The 5′ and 3′ INT1 deletion flanks were amplified by PCR using CEN.PK113-7D genomic DNA as template. The dominant marker, integration flanks and the primers used are the same as used in the methods described in the co-pending patent application no. U.S. 61/616,254 and WO2013/144257. Size of the PCR fragments was checked with standard agarose electrophoresis techniques. PCR amplified DNA fragments were purified with the NucleoMag® 96 PCR magnetic beads kit of Macherey-Nagel, according to the manual. DNA concentration was measured using the Trinean DropSense® 96 of GC biotech.

1.3 Transformation of the Fragments to S. cerevisiae

Transformation of S. cerevisiae was done as described by Gietz and Woods (2002; Transformation of the yeast by the LiAc/SS carrier DNA/PEG method. Methods in Enzymology 350: 87-96).

CEN.PK1137D (MATa URA3 HIS3 LEU2 TRP1 MAL2-8 SUC2) and the PDC1 KO strain were transformed with 1 μg of each of the amplified and purified PCR fragments. Each transformation will result in a “itaconic acid pathway” with the itaconic acid cassettes and KanMX marker integrated into the INT1 locus on the genome. Transformation mixtures were plated on YEPhD-agar (BBL Phytone peptone 20.0 g/l, Yeast Extract 10.0 g/l, Sodium Chloride 5.0 g/l, Agar 15.0 g/l and 2% glucose) containing G418 (400 μg/ml). After 3 days of incubation at 30° C., colonies appeared on the plates, whereas the negative control (i.e., no addition of DNA in the transformation experiment) resulted in blank plates. Table 2 shows an overview of the transformations that were done to both CEN.PK1137D and the PDC1 KO strain.

TABLE 2
Overview of the cassettes transformed in each transformation

| Transformation # | Position 1 | Position 2 | Position 3 | Position 4 | Position 5 | Position 6 | Position 7 |
|---|---|---|---|---|---|---|---|
| 1 | CAS117 | CAS120 | CAS133 | CAS136 | CAS124 | CAS126 | |
| 2 | CAS118 | CAS120 | CAS133 | CAS136 | CAS124 | CAS126 | |
| 3 | CAS119 | CAS120 | CAS133 | CAS136 | CAS124 | CAS126 | |
| 4 | CAS117 | CAS121 | CAS133 | CAS136 | CAS124 | CAS126 | |
| 5 | CAS117 | CAS122 | CAS133 | CAS136 | CAS124 | CAS126 | |
| 6 | CAS117 | CAS123 | CAS133 | CAS136 | CAS124 | CAS126 | |
| 7 | CAS117 | CAS120 | CAS134 | CAS136 | CAS124 | CAS126 | |
| 8 | CAS117 | CAS120 | CAS135 | CAS136 | CAS124 | CAS126 | |
| 9 | CAS117 | CAS120 | CAS133 | CAS136 | CAS125 | CAS126 | |
| 10 | CAS117 | CAS120 | CAS133 | CAS136 | CAS137 | CAS140 | |
| 11 | CAS117 | CAS120 | CAS133 | CAS136 | CAS138 | CAS140 | |
| 12 | CAS117 | CAS120 | CAS133 | CAS136 | CAS139 | CAS140 | |
| 13 | CAS117 | CAS120 | CAS133 | CAS136 | CAS137 | CAS127 | CAS141 |
| 14 | CAS117 | CAS120 | CAS133 | CAS136 | CAS137 | CAS128 | CAS141 |
| 15 | CAS117 | CAS120 | CAS133 | CAS136 | CAS137 | CAS129 | CAS141 |
| 16 | CAS117 | CAS120 | CAS133 | CAS136 | CAS137 | CAS127 | CAS142 |
| 17 | CAS117 | CAS120 | CAS133 | CAS136 | CAS137 | CAS127 | CAS143 |
| 18 | CAS117 | CAS120 | CAS144 | CAS136 | CAS124 | CAS126 | |
| 19 | CAS118 | CAS120 | CAS144 | CAS136 | CAS124 | CAS126 | |
| 20 | CAS119 | CAS120 | CAS144 | CAS136 | CAS124 | CAS126 | |
| 21 | CAS117 | CAS121 | CAS144 | CAS136 | CAS124 | CAS126 | |
| 22 | CAS117 | CAS122 | CAS144 | CAS136 | CAS124 | CAS126 | |
| 23 | CAS117 | CAS123 | CAS144 | CAS136 | CAS124 | CAS126 | |
| 24 | CAS117 | CAS120 | CAS144 | CAS136 | CAS125 | CAS126 | |
| 25 | CAS117 | CAS120 | CAS144 | CAS136 | CAS137 | CAS140 | |
| 26 | CAS117 | CAS120 | CAS144 | CAS136 | CAS138 | CAS140 | |
| 27 | CAS117 | CAS120 | CAS144 | CAS136 | CAS139 | CAS140 | |
| 28 | CAS117 | CAS120 | CAS144 | CAS136 | CAS137 | CAS127 | CAS141 |
| 29 | CAS117 | CAS120 | CAS144 | CAS136 | CAS137 | CAS128 | CAS141 |
| 30 | CAS117 | CAS120 | CAS144 | CAS136 | CAS137 | CAS129 | CAS141 |
| 31 | CAS117 | CAS120 | CAS144 | CAS136 | CAS137 | CAS127 | CAS142 |
| 32 | CAS117 | CAS120 | CAS144 | CAS136 | CAS137 | CAS127 | CAS143 |
| 33 | CAS117 | CAS120 | CAS133 | CAS136 | CAS147 | CAS140 | |
| 34 | CAS117 | CAS120 | CAS133 | CAS136 | CAS147 | CAS127 | CAS141 |
| 35 | CAS117 | CAS120 | CAS133 | CAS136 | CAS147 | CAS128 | CAS141 |
| 36 | CAS117 | CAS120 | CAS133 | CAS136 | CAS147 | CAS129 | CAS141 |
| 37 | CAS117 | CAS120 | CAS133 | CAS136 | CAS147 | CAS127 | CAS142 |
| 38 | CAS117 | CAS120 | CAS133 | CAS136 | CAS147 | CAS127 | CAS143 |
| 39 | CAS117 | CAS120 | CAS144 | CAS136 | CAS147 | CAS140 | |
| 40 | CAS117 | CAS120 | CAS144 | CAS136 | CAS147 | CAS127 | CAS141 |
| 41 | CAS117 | CAS120 | CAS144 | CAS136 | CAS147 | CAS128 | CAS141 |
| 42 | CAS117 | CAS120 | CAS144 | CAS136 | CAS147 | CAS129 | CAS141 |
| 43 | CAS117 | CAS120 | CAS144 | CAS136 | CAS147 | CAS127 | CAS142 |
| 44 | CAS117 | CAS120 | CAS144 | CAS136 | CAS147 | CAS127 | CAS143 |

1.4 Cultivation of the Transformants

Single colonies were picked and transferred to a MTP agar well containing 200 μl YEPhD-agar containing 400 μg/ml G418. For each transformation 2 to 4 colonies were used for further analysis. After 3 days of incubation of the plate at 30° C., good grown colonies were inoculated by transferring some colony material with a pin tool in a MTP plate with standard lid containing in each well 200 μL Verduyn medium (Verduyn et al., Yeast 8:501-517, 1992, where the (NH4)2SO4 was replaced with 2 g/l Urea) with a C-source based on starch and an enzyme providing release of glucose during cultivation. The MTP was incubated in a MTP shaker (INFORS HT Multitron) at 30° C., 550 rpm and 80% humidity for 72 hours. After this pre-culture phase a production phase was started by transferring 80 μl of the broth to 4 ml Verduyn media (again with the urea replacing (NH4)2SO4) with a C-source based on starch and an enzyme providing release of glucose during cultivation. After 7 days growth in the shaker at 550 rpm, 30° C. and 80% humidity the plates were centrifuged for 10 minutes at 2750 rpm in a Heraeus Multifuge 4. Supernatant was transferred to MTP plates and itaconic acid levels in the supernatant were measured with a hereafter described LC-MS method.

1.5 Detection of Itaconic Acid and Itaconate Methyl Ester

UPLC-MS/MS analysis method for the determination of itaconic acid, and other compounds of the Krebs cycle. A Waters HSS T3 column 1.7 μm, 100 mm*2.1 mm was used for the separation of itaconic, succinic, citric, iso-citric, malic and fumaric acid, as well as the possible methyl- and ethyl ester of itaconic acid with gradient elution. Eluens A consists of LC/MS grade water, containing 0.1% formic acid, and eluens B consists of acetonitrile, containing 0.1% formic acid. The flow-rate was 0.35 ml/min and the column temperature was kept constant at 40° C. The gradient started at 95% A and was increased linear to 30% B in 10 minutes, kept at 30% B for 2 minutes, then immediately to 95% A and stabilized for 5 minutes. The injection volume used was 2 ul.

A Waters Xevo API was used in electrospray (ESI) in negative ionization mode, using multiple reaction monitoring (MRM). The ion source temperature was kept at 130° C., whereas the desolvation temperature is 350° C., at a flow-rate of 500 L/hr.

For itaconic acid and the other compounds of the Krebs cycle the deprotonated molecule was fragmented with 10 eV, resulting in specific fragments from losses of H2O and CO2. The standards of reference compounds spiked in blank fermentation broth were analyzed to confirm retention time, calculate a response factor for the respective ions, and was used to calculate the concentrations in fermentation samples. All samples were diluted appropriately (5-25 fold) in eluens A to overcome ion suppression and matrix effects during LC-MS analysis. Accurate mass analysis of itaconic acid and esters of itaconic acid. To confirm the elemental composition of the compounds analyzed accurate mass analyses was performed with the same chromatographic system as described above, coupled to a LTQ orbitrap (ThermoFisher). Mass calibration was performed in constant infusion mode, using a NaTFA mixture (ref), in such a way that during the experimental set-up the accurate mass analyzed could be fitted within 2 ppm from the theoretical mass, of all compounds analyzed.

1.6 Itaconic Acid and Itaconate Methyl Ester Concentrations

Itaconic acid concentrations per pathway group and per strain group are shown in Table 3. The concentrations in the table are median values per strain or pathway group. The LC-MS analysis also detected 4-methyl itaconate in the samples and confirmed the mass and retention time with the standard. Concentrations found in the samples of 4-methyl itaconate range between 100 and 200 mg/l.

TABLE 3
Itaconic acid concentration results

| | Pathway | | | | | | | | | | | | | | | | |
|---|---|---|---|---|---|---|---|---|---|---|---|---|---|---|---|---|---|
| | 1 | | | | | | | | | 2 | | | | 3 | | | 4 |
| | Strain | | | | | | | | | | | | | | | | |
| | 1 | 2 | 3 | 4 | 5 | 6 | 7 | 8 | 9 | 10 | 11 | 12 | 13 | 14 | 16 | 17 | 15 |
| Itaconate (mg/L) | 106 | | | | | | | | | 185 | | | | 136 | | | 100 |
| | 106 | 93 | 98 | 126 | 72 | 133 | 54 | 114 | 109 | 184 | 181 | 195 | 132 | 126 | 151 | 144 | 100 |

TABLE 4
Description of sequence listinq

| Nucleic acid | Amino acid | Id* | UniProt | Organism |
|---|---|---|---|---|
| SEQ ID NO: 1 | SEQ ID NO: 2 | ITE_01 | Q0C8L2 | A. terreus |
| SEQ ID NO: 3 | SEQ ID NO: 4 | ITE_02 | | A. terreus |
| SEQ ID NO: 5 | SEQ ID NO: 6 | ITE_03 | Orf16 | A. terreus |
| SEQ ID NO: 7 | SEQ ID NO: 8 | CAD_01 | mCAD3 | A. terreus |
| SEQ ID NO: 9 | SEQ ID NO: 10 | CAD_02 | mCAD2 | A. terreus |
| SEQ ID NO: 11 | SEQ ID NO: 12 | CAD_03 | Q0C8L3 | A. terreus |
| SEQ ID NO: 13 | SEQ ID NO: 14 | CAD_04 | Q9Y7D9 | A. terreus |
| SEQ ID NO: 15 | SEQ ID NO: 16 | ACO_01 | A7A1I8 | S. cerevisiae |
| SEQ ID NO: 17 | SEQ ID NO: 18 | ACO_02 | PRPD_ECOLI | E. coli |
| SEQ ID NO: 19 | SEQ ID NO: 20 | ACO_03 | ACON2_ECOLI | E. coli |
| SEQ ID NO: 21 | SEQ ID NO: 22 | CTP_01 | Q04013 | S. cerevisiae |
| SEQ ID NO: 23 | SEQ ID NO: 24 | OTP_01 | P32332 | S. cerevisiae |
| SEQ ID NO: 25 | SEQ ID NO: 26 | PYC_01 | P32327 | S. cerevisiae |
| SEQ ID NO: 27 | SEQ ID NO: 28 | CSc_01 | CISY_YEAST | S. cerevisiae |
| SEQ ID NO: 29 | SEQ ID NO: 30 | CSc_02 | CISY_PIG | Sus scrofa |
| SEQ ID NO: 31 | SEQ ID NO: 32 | CSc_03 | C9R0Q1_ECOD1 | E. coli |
| SEQ ID NO: 33 | SEQ ID NO: 34 | ACDH67 | Q92CP2 | Listeria innocua |
| SEQ ID NO: 35 | SEQ ID NO: 36 | XFP_01 | Q6UPD8 | Lactobacillus paraplantarum. |
| SEQ ID NO: 37 | SEQ ID NO: 38 | XFP_02 | Q9AEM9 | Bifidobacterium animalis subsp. |
| | | | | lactis DSM 10140 |
| SEQ ID NO: 39 | SEQ ID NO: 40 | ACK_01 | Q1R9B8 | E. coli |
| SEQ ID NO: 41 | SEQ ID NO: 42 | PTA_01 | F5ZUJ6 | S. enterica |
| SEQ ID NO: 43 | SEQ ID NO: 44 | PTA_02 | P41790 | S. enterica |
| SEQ ID NO: 45 | SEQ ID NO: 46 | PTA_03 | P39646 | Bacillus subtilis |
| SEQ ID NO: 47 | SEQ ID NO: 48 | CTP_03 | Orf14 | A. terreus |

TABLE 5
Description of sequence listing

| SEQ ID | SEQ NAME | |
|---|---|---|
| SEQ ID NO: 49 | Sc Act1. | pro |
| SEQ ID NO: 50 | Sc TDH3. | pro |
| SEQ ID NO: 51 | Sc Tef1. | pro |
| SEQ ID NO: 52 | Sc ENO2. | pro |
| SEQ ID NO: 53 | Sc PGI1. | pro |
| SEQ ID NO: 54 | Sc FBA1. | pro |
| SEQ ID NO: 55 | Sc PGK1. | pro |
| SEQ ID NO: 56 | Sc PRE3. | pro |
| SEQ ID NO: 57 | Sc TDH1. | pro |
| SEQ ID NO: 58 | Sc ADH1. | ter |
| SEQ ID NO: 59 | Sc TDH1. | ter |
| SEQ ID NO: 60 | Sc PDC1. | ter |
| SEQ ID NO: 61 | Sc TAL1. | ter |
| SEQ ID NO: 62 | Sc TDH3. | ter |
| SEQ ID NO: 63 | Sc GPM1. | ter |
| SEQ ID NO: 64 | Sc TPI1. | ter |

TABLE 6
Description of sequence listing

| SEQ ID | SEQ ID | |
|---|---|---|
| Amino acid | Nucleic acid | SEQ NAME |
| SEQ ID NO: 65 | SEQ ID NO: 68 | Trans-aconitate 2-methyltransferase (E. coli K12) |
| SEQ ID NO: 66 | SEQ ID NO: 69 | Trans-aconitate 3-methyltransferase (S. cerevisiae) |
| SEQ ID NO: 67 | SEQ ID NO: 70 | aconitate delta-isomerase (Brucella ceti str. Cudo) |

### Example 2: Construction of Yeast Strain IAD01

### Generation of PCR Fragments

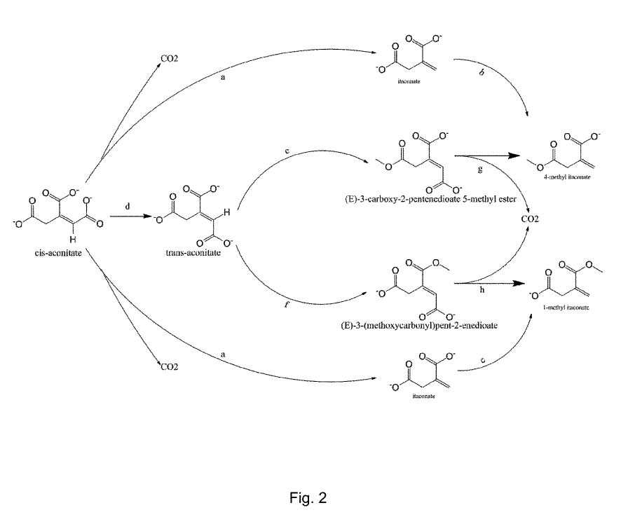

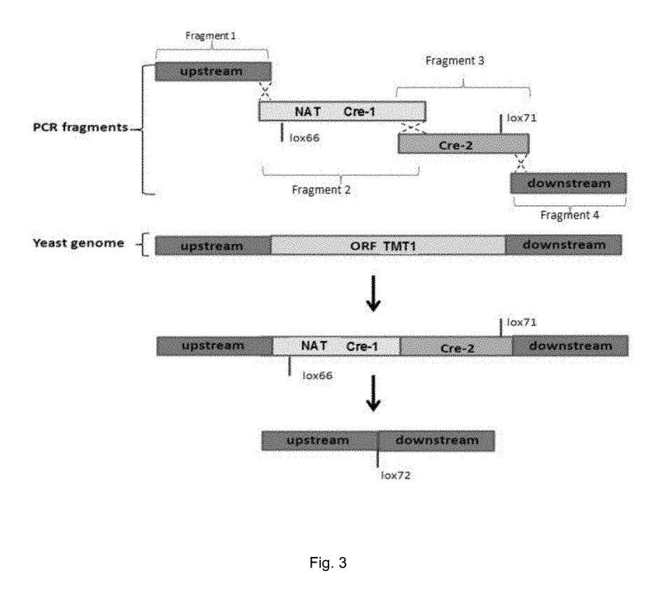

PCR fragments are generated using Phusion DNA polymerase (New England Biolabs, USA) according to manufacturer's instructions. PCR fragment 1 is generated by using genomic DNA as template (see FIG. 3). PCR fragment 2 is generated by using primer sequences designed using plasmid pSUC228 as template. Plasmid pSUC228 is a modified version of pSUC227, which is described in PCT/EP2013/055047. Plasmid pSUC227 contains a KanMX marker, which is replaced by a nourseothricin (natMX4) marker (Goldstein and McCusker, Yeast. 1999 October; 15(14):1541-53) resulting in pSUC228. PCR fragment 3 is generated by using plasmid pSUC225 (described in PCT/EP2013/055047) as template. PCR fragment 4 is generated using genomic DNA as template.

The size of the PCR fragments is checked with standard agarose electrophoresis techniques. PCR amplified DNA fragments are purified using the Zymoclean™ Gel DNA Recovery Kit (Zymo Research, Irvine, Calif., USA) according to manufacturer's instructions.

### Transformation to Strain ITA09 in Order to Construct Strain IAD01

The Saccharomyces cerevisiae strain ITA09 was constructed as described in previous Example. Strain ITA09 is obtained by transforming CEN.PK1137D strain with cassette CAS117, CAS121, CAS133, CAS136, CAS139, and CAS140 as described in patent Example 1. Strain ITA09 is used as a starting point to construct strain IAD01.

Yeast transformation is carried out by any suitable method known to persons skilled in the art. S. cerevisiae strain ITA09 is transformed with purified PCR fragments 1, 2, 3, and 4. PCR fragment 1 contains an overlap with PCR fragment 2 at its 3′ end. PCR fragment 4 contains an overlap with PCR fragment 3 at its 5′ end. PCR fragment 2 contains an overlap at its 5′ end with PCR fragment 1 and at its 3′ end with PCR fragment 3, and PCR fragment 3 contains an overlap at its 5′ end with PCR fragment 2 and at its 3′ end with PCR fragment 4, such that this allows homologous recombination of all four PCR fragments (FIG. 3). The 5′ end of PCR fragment 1 and the 3′ end of PCR fragment 4 is homologous to the TMT1 locus and enables integration of all four PCR fragments in the TMT1 locus. This results in one linear fragment consisting of PCR fragments 1 to 4 integrated in the TMT1 locus (FIG. 3).

Transformation mixtures are plated on YPD-agar (per liter: 10 grams of yeast extract, 20 grams per liter peptone, 20 grams per liter dextrose, 20 grams of agar) containing 100 μg nourseothricin (Jena Bioscience, Germany) per ml. After three to five days of growth at 30° C., individual transformants are re-streaked on fresh YPD-agar plates containing 100 μg nourseothricin per ml.

Subsequently, the marker cassette and Cre-recombinase are effectively out-recombined by the method described in PCT/EP2013/055047, resulting in deletion of the TMT1 gene and leaving a lox72 site as a result of recombination between the lox66 and lox71 sites. Due to the activity of Cre-recombinase, the NAT marker flanked by lox66 and lox71 sites, which is introduced into genomic DNA during the transformation of ITA09 is also efficiently out-recombined. The resulting NAT-markerfree strain is named IAD01. Strain IADO1 is able to grow on YPD-agar plates, but unable to grow on YPD-agar plates supplemented with 100 μg/ml nourseothricin, confirming out-recombination of the NAT marker.

### Example 3: Production of Itaconic Acid and Itaconate Methyl Ester in IADO1

3.1 Aerobic Cultivation of the Transformants

One loop of cells from a culture on a YEPhD-agar plate is transferred to a 20 ml YEPhD medium in 100 ml shake flask. After 36 hours of incubation at 30° C. and 280 rpm, 8 ml is transferred to 1 L Dasgip fermenter containing 300 ml 2× concentrated Verduyn medium [1] with a C-source based on starch and an enzyme providing release of glucose during cultivation. To control the pH, a buffer of 30 g/l MES hydrate is added and the pH is set at 5.4 with 4N KOH. The medium is supplemented with 8 mg/l biotin. Samples are taken at 40.5 and 106 hours of fermentation at 30° C., 500 rpm stirring speed and a gasflow of 10 I/h air. Itaconic acid and itaconate methyl ester levels in the supernatant are measured with a hereafter described LC-MS method. Biomass dry weight is determined by centrifuging 10 ml of whole broth for 10 minutes at 6.000 rcf in a table centrifuge, the pellet is washed with demineralized water and dried at 105° C. for 24 hours and weighed.

3.2 Detection of Itaconic Acid and Itaconate Methyl Ester

UPLC-MS/MS analysis method for the determination of itaconic acid, and other compounds of the Krebs cycle. A Waters ACQUITY UPLC HSS T3 column 1.7 μm, 150 mm*2.1 mm is used for the separation of itaconic, succinic, citric, iso-citric, malic and fumaric acid, as well as the possible methyl- and ethyl ester of itaconic acid with gradient elution. Eluents A consists of LC/MS grade water, containing 0.1% formic acid, and eluents B consists of acetonitrile, containing 0.1% formic acid. The flow-rate is 0.35 ml/min and the column temperature is kept constant at 40° C. The gradient started at 95% A, is increased linear to 21.6% B in 6 minutes, then increased to 40% B and is constant for 1.3 minutes (wash), then immediately decreased to 95% A and stabilized for 1.4 minutes (equilibration). The injection volume used is 5 ul.

A Waters Xevo API is used in electrospray (ESI) in negative ionization mode, using multiple reaction monitoring (MRM). The ion source temperature is kept at 150° C., whereas the desolvation temperature is 600° C., at a flow-rate of 700 L/hr.

For itaconic acid and the other compounds of the Krebs cycle the deprotonated molecule is fragmented with 13 eV, resulting in specific fragments from losses of H2O and CO2. The standards of reference compounds are analysed to confirm retention time, calculate a response factor for the respective ions, and is used to calculate the concentrations in fermentation samples. All samples are diluted appropriately (5-25 fold) in water to overcome ion suppression and matrix effects during LC-MS analysis. Accurate mass analysis of itaconic acid and esters of itaconic acid. To confirm the elemental composition of the compounds analyzed accurate mass analyses is performed with the same chromatographic system as described above, coupled to a LTQ orbitrap (ThermoFisher). Mass calibration is performed in constant infusion mode, using a NaTFA mixture, in such a way that during the experimental set-up the accurate mass analysed could be fitted within 2 ppm from the theoretical mass, of all compounds analysed.

3.3 Itaconic Acid and Itaconate Methyl Ester Concentrations

Itaconate methyl ester production by IADO1 is determined. Itaconic acid concentration produced by IADO1 is compared with that of ITA09.

1. A recombinant cell which is capable of producing one or more of itaconic acid, 4-methyl itaconate or 1-methyl itaconate, wherein said recombinant cell has been modified in its genome resulting in a deficiency in production of a trans-aconitate methyltransferase.

2. A recombinant cell according to claim 1 in which one or more nucleic acid sequences encoding a polypeptide are overexpressed, said polypeptide(s) being capable of catalyzing one or more of the conversions:
a. cis-aconitate to itaconate;
b. itaconate to 4-methyl itaconate;
c. itaconate to 1-methyl itaconate;
d. cis-aconitate to trans-aconitate;
e. trans-aconitate to (E)-3-carboxy-2-pentenedioate 5-methyl ester;
f. trans-aconitate to (E)-3-(methoxycarbonyl)pent-2-enedioate;
g. (E)-3-carboxy-2-pentenedioate 5-methyl ester to 4-methyl itaconate; and
h. (E)-3-(methoxycarbonyl)pent-2-enedioate to 1-methyl itaconate.

3. A recombinant cell according to claim 2 which is capable of producing 1-methyl itaconate and which comprises one or more nucleic acid sequences encoding polypeptides capable of catalyzing the conversions:
a and c; or
d, f and h.

4. A recombinant cell according to claim 2 which is capable of producing 4-methyl itaconate and which comprises one or more nucleic acid sequences encoding polypeptides capable of catalyzing the conversions:
a and b; or
d, e, and g.

5. A recombinant cell according to claim 1 which is a yeast cell.

6. A recombinant yeast cell, optionally according to claim 1, which is capable of producing itaconic acid and which overexpresses:
a nucleic acid encoding a polypeptide having cis-aconitate decarboxylase activity; and
one or more nucleic acids encoding polypeptides which separately or together catalyze a reaction towards acetyl CoA,
wherein the said recombinant microorganism has been modified in its genome resulting in a deficiency in production of a trans-aconitate methyltransferase

7. A recombinant yeast cell according to claim 6, wherein the nucleic acid encoding a polypeptide which catalyzes a reaction towards acetyl CoA is
nucleic acid sequences encoding polypeptides which together have pyruvate dehydrogenase activity;
one or more nucleic acid sequences encoding one or more polypeptides having pyruvate decarboxylase activity, acetaldehyde dehydrogenase activity and/or acetyl-CoA synthetase activity;
a nucleic acid sequence encoding a polypeptide having acetylating acetaldehyde dehydrogenase activity;
a nucleic acid sequence encoding a polypeptide having pyruvate:NADP oxidoreductase activity;
a nucleic acid encoding a polypeptide having acetate:CoA ligase (ADP-forming) activity;
a nucleic acid encoding a polypeptide ATP:acetate phosphotransferase activity and a nucleic acid encoding a polypeptide having acetyl-CoA:Pi acetyltransferase activity/phosphate acetyltransferase activity.

8. A recombinant cell according to claim 1 which overexpresses:
a nucleic acid encoding a polypeptide catalyzing conversion of citrate to cis-aconitate; and/or
a nucleic acid encoding a polypeptide having citrate synthase activity.

9. A recombinant cell according to claim 1 which overexpresses:
a nucleic acid encoding a polypeptide having pyruvate carboxylase; and/or
a nucleic acid encoding a polypeptide having PEP carboxykinase activity; and/or
a nucleic acid encoding a polypeptide having PEP carboxylase.

10. A recombinant cell according to claim 1 which overexpresses:
a nucleic acid sequence encoding a mitochondrial membrane citrate transporter.

11. A recombinant cell according to claim 1 which comprises:
a nucleic acid sequence encoding a itaconic acid transporter, a 4-methyl itaconate transporter or a 1-methyl itaconate transporter.

12. A recombinant cell according to claim 1 comprising a genetic modification resulting in reduced expression and/or activity of pyruvate decarboxylase, alcohol dehydrogenase, isocitrate dehydrogenase, alpha-ketoglutarate dehydrogenase, or succinyl-CoA ligase in the cell as compared to a cell without the genetic modification.

13. A recombinant cell according to claim 1 which is a yeast cell, optionally a S. cerevisiae cell.

14. A recombinant cell, optionally according to claim 1, which comprises, optionally overexpresses, polypeptides catalysing the following reactions:
transportation of cytosolic itaconate to extracellular itaconic acid;
conversion of cytosolic cis-aconitate to itaconate;
conversion of cytosolic citrate to cis-aconitate;
conversion of cytosolic oxaloacetate and acetyl-coenzyme-A to citrate;
conversion of cytosolic acetaldehyde, NAD, and coenzyme-A to acetyl-coenzyme-A and NADH; and
conversion of cytosolic pyruvate and bicarbonate to oxaloacetate.

15. A recombinant cell, optionally according to claim 1, which comprises, optionally overexpresses, polypeptides catalysing the following reactions:
transportation of cytosolic itaconate to extracellular itaconic acid;
conversion of cytosolic cis-aconitate to itaconate;
conversion of cytosolic citrate to cis-aconitate;
transportation of mitochondrial citrate to the cytosol;
transportation of cytosolic oxaloacetate to the mitochondria; and
conversion of cytosolic pyruvate and bicarbonate to oxaloacetate.

16. A recombinant cell, optionally according to claim 1, which comprises, optionally overexpresses, polypeptides catalysing the following reactions:
transportation of cytosolic itaconate to extracellular itaconic acid;
conversion of cytosolic cis-aconitate to itaconate;
conversion of cytosolic citrate to cis-aconitate;
conversion of cytosolic oxaloacetate and acetyl-coenzyme-A to citrate;
conversion of cytosolic acetyl-phosphate to acetyl-coenzyme-A;
conversion of xylulose-5-phosphate and phosphate to acetyl-phosphate and glyceraldehyde 3-phosphate; and
conversion of cytosolic pyruvate and bicarbonate to oxaloacetate.

17. A recombinant cell, optionally according to claim 1, which comprises, optionally overexpresses, polypeptides catalysing the following reactions:
transportation of cytosolic itaconate to extracellular itaconic acid;
conversion of cytosolic cis-aconitate to itaconate;
conversion of cytosolic citrate to cis-aconitate;
conversion of cytosolic oxaloacetate and acetyl-coenzyme-A to citrate;
conversion of cytosolic acetyl-phosphate to acetyl-coenzyme-A;
conversion of cytosolic acetate and ATP to acetyl-phosphate, ADP, and phosphate; and
conversion of cytosolic pyruvate and bicarbonate to oxaloacetate.

18. A recombinant cell according to claim 14 which is a yeast cell, optionally a Saccharomyces cerevisiae cell.

19. A recombinant cell according to claim 1 wherein said recombinant microorganism has been modified in its genome resulting in a deficiency in the production of a trans-aconitate methyltransferase comprising an amino acid sequence having at least about 30% sequence identity with SEQ ID NO: 66.

20. A process for the production of 4-methyl itaconate or 1-methyl itaconate, which process comprises fermenting a recombinant cell according to claim 1 in a suitable fermentation medium, wherein 4-methyl itaconate or 1-methyl itaconate is produced.

21. A process for the production of itaconic acid or an ester of itaconic acid, which process comprises fermenting a recombinant cell according to claim 6 in a suitable fermentation medium, wherein the itaconic acid or ester of itaconic acid is produced.

22. A process according to claim 20, wherein the itaconic acid or ester of itaconic acid is further converted into a pharmaceutical, cosmetic, food, feed or chemical product.

23. A fermentation broth comprising a itaconic acid and/or an ester of itaconate obtainable by a process according to claim 20.


SEQUENCE LISTING (70 sequences)

SEQ ID NO: 1 - DNA, 1212 bp, Aspergillus terreus
  CDS (1)..(1212)
atgggtcacggtgacactgaatctccaaacccaaccaccaccactgaaggttctggtcaaaacgaacctgaaaagaagggtcgtgacattccattatggagaaagtgtgttatcactttcgttgtttcctggatgactttggttgtcactttctcctccacctgtttgttgccagctgctccagaaattgctaacgaattcgatatgaccgtcgaaaccattaacatttccaacgctggtgttttggttgccatgggttactcttctttgatctggggtccaatgaacaaattggttggtagaagaacctcttacaacttggccatctccatgttgtgtgcctgttctgctggtactgctgctgccatcaacgaagaaatgttcattgctttccgtgtcttgtctggcttgaccggtacttctttcatggtttccggtcaaaccgtcttggctgatatctttgaaccagtttacagaggtactgctgtcggtttcttcatggctggtactctatccggtccagccattggtccatgtgtcggtggtgtcattgtcactttcacctcctggagagttatcttctggttacaattgggtatgtctggtttaggtttggttttgtctctattattcttcccaaagatcgaaggtaactctgaaaaggtttctactgctttcaagccaaccactttggtcaccatcatctccaagttctctccaaccgatgtcttgaagcaatgggtttacccaaatgtctttttggctgatttgtgttgtggtttgttggccatcactcaatactccatcttgacttctgccagagctatcttcaactccagattccatttgaccaccgctttggtttccggtttattctacttggctccaggtgctggtttcttgattggttctttggttggtggtaaattgtctgacagaaccgtcagaagatacattgtcaagagaggtttcagattacctcaagacagattgcactctggtttgatcactttgtttgctgtcttgccagctggtactttgatctacggttggactttgcaagaggacaagggtgacatggttgttccaatcattgctgctttctttgctggttggggtttgatgggttctttcaactgtttgaacacctacgttgctggtttattccacactttgatctacttgttcccattgtgtacctgtccacaataa

SEQ ID NO: 2 - protein, 403 aa, Aspergillus terreus
MGHGDTESPNPTTTTEGSGQNEPEKKGRDIPLWRKCVITFVVSWMTLVVTFSSTCLLPAAPEIANEFDMTVETINISNAGVLVAMGYSSLIWGPMNKLVGRRTSYNLAISMLCACSAGTAAAINEEMFIAFRVLSGLTGTSFMVSGQTVLADIFEPVYRGTAVGFFMAGTLSGPAIGPCVGGVIVTFTSWRVIFWLQLGMSGLGLVLSLLFFPKIEGNSEKVSTAFKPTTLVTIISKFSPTDVLKQWVYPNVFLADLCCGLLAITQYSILTSARAIFNSRFHLTTALVSGLFYLAPGAGFLIGSLVGGKLSDRTVRRYIVKRGFRLPQDRLHSGLITLFAVLPAGTLIYGWTLQEDKGDMVVPIIAAFFAGWGLMGSFNCLNTYVAGLFHTLIYLFPLCTCPQ

SEQ ID NO: 3 - DNA, 1203 bp, Aspergillus terreus
  CDS (1)..(1203)
atgggtgaattgaaggaaatcttgaagcaaagataccatgaattgttggactggaacgtcaaggctccacacgttccattgtctcaaagattgaagcacttcacctggtcttggtttgcttgtaccatggccactggtggtgtcggttctacctgtttgttgccagctgctccagaaattgctaacgaattcgacatgaccgttgaaaccatcaacatctccaatgctggtgttttggttgccatgggttactcttctttgatctggggtccaatgaacaaattggttggtcgtcgtacctcttacaacttggccatttccatgttgtgtgcttgttctgctggtactgctgctgccattaacgaagaaatgttcattgctttcagagttttgtccggtttgactggtacttctttcatggtttctggtcaaaccgttttggctgatatctttgaacctgtttacagaggtactgctgtcggtttcttcatggccggtactttgtccggtccagccattggtccatgtgtcggtggtgtcattgtcactttcacctcctggagagtcattttctggttacaattgggtatgtccggtttgggtttagtcttgtctctattattcttcccaaagatcgaaggtaactctgaaaaggtttccactgctttcaagccaaccactttggtcaccatcatctccaagttctctccaaccgatgtcttgaagcaatgggtttacccaaacgtctttttggctgacttgtgttgtggtctattagctatcactcaatactccattttgacctctgccagagccattttcaactccagattccacttgaccactgctttggtttccggtttattctacttggctccaggtgctggtttcttgatcggttctttggttggtggtaaattgtctgacagaaccgtcagaagatacatcgtcaagagaggtttcagattgcctcaagacagattgcactctggtttgatcactttgtttgctgtcttaccagctggtactttgatctacggttggactttgcaagaagataagggtgacatggttgttccaatcattgctgctttcttcgctggttggggtttgatgggttctttcaactgtttgaacacctacgttgctggtttattccacactttgatctacttgttcccattatgtacctgtccacaataa

SEQ ID NO: 4 - protein, 400 aa, Aspergillus terreus
MGELKEILKQRYHELLDWNVKAPHVPLSQRLKHFTWSWFACTMATGGVGSTCLLPAAPEIANEFDMTVETINISNAGVLVAMGYSSLIWGPMNKLVGRRTSYNLAISMLCACSAGTAAAINEEMFIAFRVLSGLTGTSFMVSGQTVLADIFEPVYRGTAVGFFMAGTLSGPAIGPCVGGVIVTFTSWRVIFWLQLGMSGLGLVLSLLFFPKIEGNSEKVSTAFKPTTLVTIISKFSPTDVLKQWVYPNVFLADLCCGLLAITQYSILTSARAIFNSRFHLTTALVSGLFYLAPGAGFLIGSLVGGKLSDRTVRRYIVKRGFRLPQDRLHSGLITLFAVLPAGTLIYGWTLQEDKGDMVVPIIAAFFAGWGLMGSFNCLNTYVAGLFHTLIYLFPLCTCPQ

SEQ ID NO: 5 - DNA, 1464 bp, Aspergillus terreus
  CDS (1)..(1464)
atgggtagaggtgacactgaatctccaaacccagctaccacctctgaaggttctggtcaaaacgaacctgaaaagaagggtcgtgatatcccattatggagaaagtgtgttatcacctttgttgtttcctggatgactttggttgtcactttctcttccacctgtttgttgccagctgctccagaaattgccaacgaattcgacatgaccgtcgaaaccattaacatctccaacgctggtgttttggttgccatgggttactcttctttgatctggggtccaatgaacaaattggtcggtagaagaacctcttacaacttggccatctccatgttgtgtgcctgttccgctggtactgctgctgccatcaacgaaaagatgttcattgctttcagagttttgtctggtctgaccggtacttctttcatggtttccggtcaaaccgtcttggctgacatctttgaaccagtctacagaggtactgctgtcggtttcttcatggctggtactttatctggtccagccattgcttgtgttggtggtgtcattgtcactttcacctcctggagagtcattttctggttacaattgggtatgtctggtttgggtttagtcttgtctctattattcttcccaaagattgaaggtacttctgaaaaggtttccactgctttcaagccaaccactttggtttccatcatctccaagttctctccaaccgatgtcttgaagcaatgggtttacccaaatgttttcttggctgtctctgcttgggaaatctgtccattgcacttgttggaaaccaaatgttcctgtagaaagcaaaaggatttgtgttgtggtttgttggccatcactcaatactccatcttgacctctgccagagctatcttcaactccagattccacttgaccactgctttggtttccggtttattctacttggctccaggtgctggtttcttgatcggttctttggttggtggtaaattgtctgacagaaccgtccgtcgttacatcgtcaagagaggtttcagattacctcaagacagattgcactctggtttgatcactttgtttgctgtcttgccagctggtactttgatctacggttggactttacaagaagataagggtggtatggttgtcccaatcattgctgctttctttgctggttggggtttgatgggttctttcaactgtttgaacacctacgttgccgttgaagctttgccaagaaacagatctgctgtcattgctggtaagtacatgattcaatactctttctccgctggttcttctgctttggttgttccagtcattgacgctttgggtgtcggttggactttcactctatgtgttgttgcttccaccattgctggtttgatcactgctgccattgccagatggggtatcaacatgcaaagatgggctgaaagagctttcaacttgccaacacagtaa

SEQ ID NO: 6 - protein, 487 aa, Aspergillus terreus
MGRGDTESPNPATTSEGSGQNEPEKKGRDIPLWRKCVITFVVSWMTLVVTFSSTCLLPAAPEIANEFDMTVETINISNAGVLVAMGYSSLIWGPMNKLVGRRTSYNLAISMLCACSAGTAAAINEKMFIAFRVLSGLTGTSFMVSGQTVLADIFEPVYRGTAVGFFMAGTLSGPAIACVGGVIVTFTSWRVIFWLQLGMSGLGLVLSLLFFPKIEGTSEKVSTAFKPTTLVSIISKFSPTDVLKQWVYPNVFLAVSAWEICPLHLLETKCSCRKQKDLCCGLLAITQYSILTSARAIFNSRFHLTTALVSGLFYLAPGAGFLIGSLVGGKLSDRTVRRYIVKRGFRLPQDRLHSGLITLFAVLPAGTLIYGWTLQEDKGGMVVPIIAAFFAGWGLMGSFNCLNTYVAVEALPRNRSAVIAGKYMIQYSFSAGSSALVVPVIDALGVGWTFTLCVVASTIAGLITAAIARWGINMQRWAERAFNLPTQ

SEQ ID NO: 7 - DNA, 1476 bp, Aspergillus terreus
  CDS (1)..(1476)
atgaccaagcaatctgctgactccaatgccaagtctggtgttacttctgaaatctgtcactgggcttctaacttggctaccgatgacatcccatctgatgtcttggaaagagctaagtacttgatcttggacggtattgcttgtgcttgggttggtgccagagttccatggtctgaaaagtacgttcaagctaccatgtccttcgaacctccaggtgcttgtcgtgtcattggttacggtcaaaaattgggtcctgttgctgctgccatgaccaactctgcctttattcaagctactgaattggacgactaccactctgaagctccattacattccgcttccattgtcttaccagctgtctttgctgcttctgaagttttggctgaacaaggtaagactatctctggtatcgatgtcatcttggctgccattgtcggtttcgaatccggtccaagaatcggtaaggccatctacggttccgatttgttgaacaacggttggcattgtggtgccgtttacggtgccccagctggtgctttggctaccggtaagctattaggtttgactccagactccatggaagatgctttgggtattgcctgtacccaagcttgtggtttgatgtccgctcaatacggtggtatggtcaagagagtccaacacggtttcgctgccagaaacggtttgttgggtggtttgttggctcacggtggttacgaagctatgaagggtgttttggaaagatcttacggtggtttcttgaagatgttcaccaagggtaacggtagagaaccaccatacaaggaagaagaagttgttgctggtttaggttctttctggcacactttcaccatcagaatcaaattgtacgcttgttgtggtttagtccacggtccagttgaagccatcgaaaacttgcaaggtagatacccagaattattgaacagagctaacttgtccaacatcagacacgttcacgttcaattgtccactgcttctaactctcactgtggttggatcccagaagaaagaccaatttcttccattgctggtcaaatgtccgttgcttacattttggctgttcaattggttgaccaacaatgtttgttgtctcaattctctgaattcgatgacaacttggaaagaccagaagtctgggacttggccagaaaggttacctcttctcaatctgaagaattcgaccaagatggtaactgtctatccgctggtcgtgtcagaatcgaattcaacgacggttcttccatcactgaatctgttgaaaagccattgggtgtcaaggaaccaatgccaaacgaaagaattttgcacaaatacagaactttggctggttccgtcactgacgaatccagagtcaaggaaattgaagatttggttttgggtttagatcgtttgactgacatctctccattattggaattgttgaactgtccagtcaaatctccattcgggatctaa

SEQ ID NO: 8 - protein, 491 aa, Aspergillus terreus
MTKQSADSNAKSGVTSEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQGRYPELLNRANLSNIRHVHVQLSTASNSHCGWIPEERPISSIAGQMSVAYILAVQLVDQQCLLSQFSEFDDNLERPEVWDLARKVTSSQSEEFDQDGNCLSAGRVRIEFNDGSSITESVEKPLGVKEPMPNERILHKYRTLAGSVTDESRVKEIEDLVLGLDRLTDISPLLELLNCPVKSPFGI

SEQ ID NO: 9 - DNA, 1479 bp, Aspergillus terreus
  CDS (1)..(1479)
atgaccaagcaatctgctgactccaatgccaagtctggtgtcacttccgaaatctgtcactgggcttccaacttggctactgacgacattccatctgatgtcttggaaagagccaagtacttgattttggacggtattgcctgtgcttgggttggtgctcgtgttccatggtctgaaaagtacgttcaagctaccatgtccttcgaacctccaggtgcttgtcgtgtcatcggttacggtcaaaaattgggtccagttgctgctgccatgaccaactctgcctttattcaagccactgaattggatgactaccactctgaagctccattgcactctgcttccattgttctaccagctgttttcgctgcttctgaagtcttggctgaacaaggtaagaccatctctggtatcgatgttatcttagctgccattgtcggtttcgaatctggtccaagaatcggtaaggccatctacggttctgacttgttgaacaacggttggcattgtggtgccgtttacggtgctccagctggtgctttggctaccggtaagttgttgggtttgactccagactccatggaagatgctttgggtatcgcttgtacccaagcttgtggtttgatgtctgctcaatacggtggtatggttaagagagttcaacatggtttcgctgccagaaacggtctattaggtggtttgttggctcacggtggttacgaagctatgaagggtgtcttggaaagatcttacggtggtttcttgaagatgttcaccaagggtaacggtagagaacctccatacaaggaagaagaagttgttgccggtttaggttctttctggcacactttcaccatcagaatcaaattgtacgcttgttgtggtttagtccacggtccagttgaagccattgaaaacttacaaggtcgttacccagaattgttgaacagagctaacttgtccaacatcagacacgttcacgttcaattatccactgcttccaactctcactgtggttggattccagaagaaagaccaatctcctccattgctggtcaaatgtctgttgcttacattttggctgtccaattggttgaccaacaatgtttgttgtctcaattctccgaattcgatgacaacttggaaagaccagaagtctgggatttggctagaaaggtcacctcttctcaatctgaagaatttgaccaagatggtaactgtttgtctgctggtagagtcagaattgaattcaacgacggttcttccatcactgaatccgttgaaaagccattaggtgtcaaggaaccaatgccaaacgaaagaatcttgcacaaatacagaactttggctggttccgtcactgacgaatccagagtcaaggaaatcgaagatttggttttgggtttggacagattgaccgatatctctccattattggaattgttgaactgtccagtcaaatctccattgggtatcaagtaa

SEQ ID NO: 10 - protein, 492 aa, Aspergillus terreus
MTKQSADSNAKSGVTSEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQGRYPELLNRANLSNIRHVHVQLSTASNSHCGWIPEERPISSIAGQMSVAYILAVQLVDQQCLLSQFSEFDDNLERPEVWDLARKVTSSQSEEFDQDGNCLSAGRVRIEFNDGSSITESVEKPLGVKEPMPNERILHKYRTLAGSVTDESRVKEIEDLVLGLDRLTDISPLLELLNCPVKSPLGIK

SEQ ID NO: 11 - DNA, 1473 bp, Aspergillus terreus
  CDS (1)..(1473)
atgaccaagcaatctgctgactccaacgccaagtctggtgtcactgctgaaatctgtcactgggcttccaacttggccaccgatgacattccatctgacgtcttggaaagagccaagtacttgatcttggacggtattgcttgtgcttgggttggtgctcgtgttccatggtctgaaaaatacgttcaagctaccatgtcctttgaacctccaggtgcttgtcgtgttatcggttacggtcaaaaattgggtcctgttgctgctgccatgaccaactctgctttcatccaagctactgaattggatgactaccactctgaagctccattgcactctgcttccattgtcttgccagctgttttcgctgcttctgaagtcttggctgaacaaggtaagaccatctccggtatcgatgttatcttggctgccattgtcggtttcgaatctggtccaagaattggtaaggccatctacggttctgatttgttgaacaacggttggcattgtggtgctgtctacggtgctccagctggtgctttggccactggtaagttgttgggtttgactccagactccatggaagatgctttaggtattgcttgtacccaagcttgtggtttgatgtccgctcaatacggtggtatggtcaagagagttcaacatggtttcgctgccagaaacggtttgttgggtggtctattagcttacggtggttacgaagctatgaagggtgttttggaaagatcttacggtggtttcttgaagatgttcaccaagggtaacggtagagaaccaccatacaaggaagaagaagttgttgccggtttgggttctttctggcacactttcaccatcagaatcaaattatacgcttgttgtggtttggtccacggtccagttgaagccatcgaaaagttgcaaagaagatacccagaattattgaacagagctaacttgtctaacatcagacacgtttacgtccaattgtccactgcttccaactctcactgtggttggatcccagaagaaagaccaatttcttccattgctggtcaaatgtccgttgcttacatcttagctgttcaattggttgaccaacaatgtttgttggctcaattctctgaattcgatgacaacttggaaagaccagaagtctgggacttggccagaaaggttactccatctcactctgaagaatttgaccaagatggtaactgtttgtctgctggtcgtgtcagaattgaattcaacgacggttcctctgttactgaaaccgtcgaaaagccattaggtgtcaaggaaccaatgccaaatgaaagaatcttgcacaagtacagaactttggccggttccgttaccgacgaatccagagtcaaggaaattgaagatttggtcttgtctctagacagattgaccgatatcactccattgttggaattattgaactgtccagtcaaatctccacttgtgtaa

SEQ ID NO: 12 - protein, 490 aa, Aspergillus terreus
MTKQSADSNAKSGVTAEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAYGGYEAMKGVLERSYGGFLKMFTKGNGREPPYKEEEVVAGLGSFWHTFTIRIKLYACCGLVHGPVEAIEKLQRRYPELLNRANLSNIRHVYVQLSTASNSHCGWIPEERPISSIAGQMSVAYILAVQLVDQQCLLAQFSEFDDNLERPEVWDLARKVTPSHSEEFDQDGNCLSAGRVRIEFNDGSSVTETVEKPLGVKEPMPNERILHKYRTLAGSVTDESRVKEIEDLVLSLDRLTDITPLLELLNCPVKSPLV

SEQ ID NO: 13 - DNA, 1473 bp, Aspergillus terreus
  CDS (1)..(1473)
atgaccaagcaatctgctgactccaatgctaagtctggtgttactgctgaaatctgtcactgggcttccaacttggccaccgatgacattccaccagatgtcttggaaagagctaagtacttgatcttggacggtattgcttgtgcctgggttggtgctcgtgttccatggtctgaaaaatacgttcaagctaccatgtctttcgaacctccaggtgcttgtcgtgtcatcggttacggtcaaaaattgggtcctgttgctgctgctatgaccaactctgctttcatccaagctactgaattggacgactaccactctgaagctccattacattccgcttccattgttttgccagctgtctttgctgcttccgaagtcttggctgaacaaggtaagaccatttctggtattgccgttatcttggccgctattgttggtttcgaatctggtccaagaatcggtaaggccatctacggttctgacttgttgaacaacggttggcactgtggtgctgtttacggtgccccagccggtgctttggctactggtaagttgttgggtttgactccagactccatggaagatgctttgggtattgcttgtacccaagcttgtggtttgatgtctgctcaatacggtggtatggtcaagagagtccaacatggttttgctgccagaaacggtctattaggtggtttgttggctcacggtggttacgaagctatgaagggtgttttggaaagatcttacggtggtttcttgaagatgttcaccaagggtaacggtagagaaccaccatacaaggaagaagaagttgttgccggtttgggttctttctggcacactttcaccatcagaatcaaattgtacgcttgttgtggtttagtccacggtccagttgaagccattgaaaacttacaaagaagatacccagaattattgaacagagccaacttgtccaacatcagacacgtccacgtccaattgtccactgcttctaactcccactgtggttggatcccagaagaaagaccaatctcttccattgctggtcaaatgtctgttgcctacatcttggctgttcaattggtcgaccaacaatgtttgttggctcaattctctgaattcgatgacaacttggaaagaccagaagtctgggacttggccagaaaggttaccccatctcactctgaagaattcgaccaagatggtaactgtttgtccgctggtcgtgtcagaattgaattcaacgatggttcctccgttactgaaactgtcgaaaagccattgggtgtcaaggaaccaatgccaaacgaaagaatcttgcacaagtacagaactttagctggttccgttaccgatgaaaccagagtcaaggaaatcgaagatttggttttgtctctagacagattgactgacatctctccattattggaattgttgaactgtccagtcaaatctccacttgtgtaa

SEQ ID NO: 14 - protein, 490 aa, Aspergillus terreus
MTKQSADSNAKSGVTAEICHWASNLATDDIPPDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIAVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQRRYPELLNRANLSNIRHVHVQLSTASNSHCGWIPEERPISSIAGQMSVAYILAVQLVDQQCLLAQFSEFDDNLERPEVWDLARKVTPSHSEEFDQDGNCLSAGRVRIEFNDGSSVTETVEKPLGVKEPMPNERILHKYRTLAGSVTDETRVKEIEDLVLSLDRLTDISPLLELLNCPVKSPLV

SEQ ID NO: 15 - DNA, 2289 bp, Saccharomyces cerevisiae
  CDS (1)..(2289)
atgactgtttccaacttgaccagagactccaaggttaaccaaaacttgttggaagatcattctttcatcaactacaagcaaaatgtcgaaactttggatatcgtcagaaagagattgaacagaccattcacctacgctgaaaagattttgtacggtcacttggatgacccacacggtcaagatatccaaagaggtgtctcctacttgaaactaagaccagatcgtgttgcttgtcaagatgctactgctcaaatggctatcttacaattcatgtccgctggtttgcctcaagttgccaagccagtcaccgtccactgtgaccatttgatccaagctcaagtcggtggtgaaaaggacttgaagagagccattgacttgaacaaggaagtctacgacttcttggcttctgccactgctaaatacaacatgggtttctggaagccaggttccggtatcatccaccaaatcgttttggaaaactatgccttcccaggtgctttgatcatcggtactgactcccacactccaaatgccggtggtctaggtcaattggccatcggtgttggtggtgctgatgctgttgacgtcatggctggtagaccatgggaattgaaggctccaaagattttgggtgttaagttgaccggtaagatgaacggttggacttctccaaaggacatcatcttgaaattggctggtatcactactgttaagggtggtactggtaagattgtcgaatactttggtgacggtgtcgacactttctctgctaccggtatgggtaccatctgtaacatgggtgctgaaattggtgccaccacttctgttttcccattcaacaaatccatgattgaatacttggaagctaccggtagaggtaagattgctgatttcgctaagttataccacaaggacttgttgtctgccgacaaggacgctgaatacgatgaagttgtcgaaattgacttgaacactttggaaccatacatcaacggtccattcaccccagatttggctaccccagtttctaagatgaaggaagttgccgttgctaacaactggccattagatgttagagttggtttgattggttcttgtaccaactcctcttacgaagatatgtccagatctgcttccattgtcaaggatgctgctgctcacggtttgaaatctaagaccatcttcactgttaccccaggttctgaacaaatcagagccaccatcgaacgtgacggtcaattggaaactttcaaggaatttggtggtattgtcttggctaacgcttgtggtccatgtattggtcaatgggacagaagagatatcaagaagggtgacaagaacaccatcgtttcctcttacaacagaaacttcacttctagaaacgatggtaacccacaaacccacgcctttgttgcttctccagaattagtcactgctttcgctattgctggtgacttgagattcaacccattaaccgacaaattgaaggacaaggacggtaacgaatttatgttgaagcctcctcatggtgatggtttaccacaaagaggttacgatgctggtgaaaacacctaccaagctccaccagccgacagatccaccgtcgaagtcaaggtttctccaacttctgacagattacaattgttgaaacctttcaagccatgggatggtaaggacgctaaggacatgccaatcttaatcaaggctgttggtaagactaccaccgaccacatttccatggctggtccatggttgaaatacagaggtcacttggaaaacatctccaacaactacatgattggtgccattaatgccgaaaacaagaaggctaactgtgtcaagaacgtttacactggtgaatacaagggtgttccagacactgccagagactacagagatcaaggtatcaaatgggttgtcatcggtgacgaaaacttcggtgaaggttcttctcgtgaacacgctgctttggaaccaagattcttgggtggtttcgctattattaccaaatctttcgctcgtattcacgaaaccaacttgaagaagcaaggtctattgccattgaacttcaagaacccagccgactacgacaagatcaacccagatgacagaattgacatcttaggtttggctgaattggctccaggtaagccagtcaccatgagagttcacccaaagaacggtaagccatgggatgctgtcttgactcacactttcaacgatgaacaaatcgaatggttcaaatacggttctgctttgaacaagatcaaggctgatgaaaagaagtaa

SEQ ID NO: 16 - protein, 762 aa, Saccharomyces cerevisiae
MTVSNLTRDSKVNQNLLEDHSFINYKQNVETLDIVRKRLNRPFTYAEKILYGHLDDPHGQDIQRGVSYLKLRPDRVACQDATAQMAILQFMSAGLPQVAKPVTVHCDHLIQAQVGGEKDLKRAIDLNKEVYDFLASATAKYNMGFWKPGSGIIHQIVLENYAFPGALIIGTDSHTPNAGGLGQLAIGVGGADAVDVMAGRPWELKAPKILGVKLTGKMNGWTSPKDIILKLAGITTVKGGTGKIVEYFGDGVDTFSATGMGTICNMGAEIGATTSVFPFNKSMIEYLEATGRGKIADFAKLYHKDLLSADKDAEYDEVVEIDLNTLEPYINGPFTPDLATPVSKMKEVAVANNWPLDVRVGLIGSCTNSSYEDMSRSASIVKDAAAHGLKSKTIFTVTPGSEQIRATIERDGQLETFKEFGGIVLANACGPCIGQWDRRDIKKGDKNTIVSSYNRNFTSRNDGNPQTHAFVASPELVTAFAIAGDLRFNPLTDKLKDKDGNEFMLKPPHGDGLPQRGYDAGENTYQAPPADRSTVEVKVSPTSDRLQLLKPFKPWDGKDAKDMPILIKAVGKTTTDHISMAGPWLKYRGHLENISNNYMIGAINAENKKANCVKNVYTGEYKGVPDTARDYRDQGIKWVVIGDENFGEGSSREHAALEPRFLGGFAIITKSFARIHETNLKKQGLLPLNFKNPADYDKINPDDRIDILGLAELAPGKPVTMRVHPKNGKPWDAVLTHTFNDEQIEWFKYGSALNKIKADEKK

SEQ ID NO: 17 - DNA, 1452 bp, Escherichia coli
  CDS (1)..(1452)
atgtccgctcaaatcaacaacatcagaccagaatttgacagagaaattgtcgatatcgttgactacgtcatgaactacgaaatttcttccaaggttgcttacgacactgctcactactgtttgttggacactttaggttgtggtttggaagctttggaatacccagcctgtaagaaattgttgggtccaattgtcccaggtaccgttgttccaaatggtgtcagagttccaggtactcaattccaattggacccagttcaagctgctttcaacatcggtgccatgatcagatggttagatttcaacgacacctggttagctgctgaatggggtcacccatctgacaacttgggtggtatcttggccactgctgactggttatccagaaacgctgttgcttccggtaaggctccattgaccatgaagcaagtcttgactgccatgatcaaggctcacgaaatccaaggttgtattgctttggaaaactctttcaaccgtgtcggtttggaccatgtcttgttggtcaaggttgcctccactgctgttgttgctgaaatgttgggtttgaccagagaagaaatcttgaacgccgtttccttggcttgggttgatggtcaatctctaagaacctacagacacgccccaaacaccggtaccagaaagtcctgggctgctggtgatgctacttccagagctgtcagattggctttgatggccaagaccggtgaaatgggttacccatctgctttgactgctccagtctggggtttctacgatgtctctttcaaaggtgaatctttcagattccaaagaccttacggttcttacgttatggaaaacgtcttattcaagatttctttcccagctgaattccactctcaaaccgctgttgaagctgctatgactttatacgaacaaatgcaagctgccggtaagactgctgctgacattgaaaaggtcaccatcagaacccacgaagcttgtatcagaattattgacaagaagggtcctttgaacaacccagctgatcgtgaccattgtatccaatacatggttgccatcccattattgtttggtagattgactgctgctgactacgaagataatgttgctcaagacaagagaattgatgctttgagagaaaagatcaactgtttcgaagatccagctttcaccgctgattaccacgacccagaaaagagagccattgccaacgccatcactttggaattcactgacggtaccagatttgaagaagttgttgtcgaatacccaattggtcacgctcgtcgtcgtcaagatggtatcccaaaattggtcgataaattcaagatcaacttggccagacaattcccaaccagacaacaacaaagaatcttggaagtttctttggacagagctagattggaacaaatgccagtcaacgaatacttggacttgtacgttatttaa

SEQ ID NO: 18 - protein, 483 aa, Escherichia coli
MSAQINNIRPEFDREIVDIVDYVMNYEISSKVAYDTAHYCLLDTLGCGLEALEYPACKKLLGPIVPGTVVPNGVRVPGTQFQLDPVQAAFNIGAMIRWLDFNDTWLAAEWGHPSDNLGGILATADWLSRNAVASGKAPLTMKQVLTAMIKAHEIQGCIALENSFNRVGLDHVLLVKVASTAVVAEMLGLTREEILNAVSLAWVDGQSLRTYRHAPNTGTRKSWAAGDATSRAVRLALMAKTGEMGYPSALTAPVWGFYDVSFKGESFRFQRPYGSYVMENVLFKISFPAEFHSQTAVEAAMTLYEQMQAAGKTAADIEKVTIRTHEACIRIIDKKGPLNNPADRDHCIQYMVAIPLLFGRLTAADYEDNVAQDKRIDALREKINCFEDPAFTADYHDPEKRAIANAITLEFTDGTRFEEVVVEYPIGHARRRQDGIPKLVDKFKINLARQFPTRQQQRILEVSLDRARLEQMPVNEYLDLYVI

SEQ ID NO: 19 - DNA, 2598 bp, Escherichia coli
  CDS (1)..(2598)
atgttggaagaatacagaaagcatgttgctgaaagagctgctgaaggtattgctccaaagccattggacgctaaccaaatggccgctttggttgaattgttgaagaacccaccagccggtgaagaagaattcttgttggatttgttgaccaacagagttcctcctggtgttgacgaagccgcttacgtcaaggctggtttcttggctgccattgccaagggtgaagctaagtctcctttgttgaccccagaaaaggccatcgaattattgggtaccatgcaaggtggttacaacattcacccattgattgacgctctagacgatgctaagttggctccaattgctgccaaggctctatcccacactttgttgatgttcgacaacttctacgatgtcgaagaaaaggccaaggccggtaacgaatacgctaagcaagttatgcaatcctgggctgatgctgaatggttcttgaacagaccagctttggctgaaaaattgactgtcaccgttttcaaggtcactggtgaaaccaacaccgatgacttgtctccagctccagatgcttggtccagaccagatatcccattgcacgctttggccatgttgaaaaatgctcgtgaaggtattgaaccagaccaaccaggtgttgtcggtccaatcaagcaaatcgaagctttgcaacaaaaaggtttcccattggcttacgtcggtgatgttgtcggtaccggttcttccagaaagtctgctaccaactctgttttatggttcatgggtgatgatatcccacacgttccaaacaagagaggtggtggtttgtgtttgggtggtaagatcgccccaattttcttcaacaccatggaagatgccggtgctttgccaattgaagtcgatgtctccaacttgaacatgggtgacgtcattgatgtttacccatacaagggtgaagtcagaaaccacgaaactggtgaattgttggctacctttgaattaaagactgacgtcttgattgacgaagtcagagctggtggtagaatcccattgatcatcggtagaggtttgactaccaaggccagagaagctttaggtttgcctcactccgatgttttcagacaagctaaggatgtcgctgaatctgacagaggtttctccttggcccaaaagatggttggtagagcttgtggtgtcaagggtatcagaccaggtgcttactgtgaaccaaagatgacttccgttggttctcaagacaccactggtccaatgaccagagatgaattgaaggacttggcttgtttgggtttctccgctgacttggttatgcaatctttctgtcacactgctgcttacccaaagccagttgacgtcaacacccatcacactctaccagacttcatcatgaaccgtggtggtgtttctttgcgtccaggtgacggtgtcattcactcctggttaaacagaatgttgttgccagacaccgttggtaccggtggtgactctcacacccgtttcccaatcggtatttctttcccagccggttccggtttggttgcctttgctgccgctactggtgtcatgccattagacatgccagaatctgttttggtcagattcaagggtaagatgcaaccaggtatcactttgagagacttagtccacgctatcccattatacgccatcaagcaaggtttgttgactgtcgaaaagaagggtaagaaaaatattttctctggtcgtattttggaaatcgaaggtttgccagatttgaaggtcgaacaagcctttgaattgactgatgcttctgctgaaagatctgccgctggttgtaccatcaaattgaacaaggaacctatcatcgaatacttgaactccaacattgtcttattgaaatggatgattgctgaaggttacggtgacagaagaactttggaaagaagaatccaaggtatggaaaaatggttagctaacccagaattgttggaagctgacgctgatgctgaatacgctgctgttatcgatatcgatttggctgacatcaaggaaccaatcctatgtgccccaaatgacccagatgacgctagaccattatctgctgtccaaggtgaaaagattgacgaagtctttatcggttcttgtatgaccaacatcggtcatttcagagctgctggtaagttgttggacgctcacaagggtcaattgccaaccagattatgggttgccccaccaactagaatggacgctgctcaattgaccgaagaaggttactactctgttttcggtaaatctggtgcccgtattgaaattccaggttgttccttgtgtatgggtaaccaagctagagttgctgacggtgctaccgttgtttccacttctaccagaaacttcccaaacagattaggtactggtgccaacgttttcttggcttctgctgaattggctgctgttgctgctttgatcggtaaattgccaactccagaagaataccaaacttacgttgctcaagtcgacaagactgctgttgacacctacagatacttgaacttcaaccaattgtctcaatacactgaaaaggctgacggtgttatcttccaaactgcggtttaa

SEQ ID NO: 20 - protein, 865 aa, Escherichia coli
MLEEYRKHVAERAAEGIAPKPLDANQMAALVELLKNPPAGEEEFLLDLLTNRVPPGVDEAAYVKAGFLAAIAKGEAKSPLLTPEKAIELLGTMQGGYNIHPLIDALDDAKLAPIAAKALSHTLLMFDNFYDVEEKAKAGNEYAKQVMQSWADAEWFLNRPALAEKLTVTVFKVTGETNTDDLSPAPDAWSRPDIPLHALAMLKNAREGIEPDQPGVVGPIKQIEALQQKGFPLAYVGDVVGTGSSRKSATNSVLWFMGDDIPHVPNKRGGGLCLGGKIAPIFFNTMEDAGALPIEVDVSNLNMGDVIDVYPYKGEVRNHETGELLATFELKTDVLIDEVRAGGRIPLIIGRGLTTKAREALGLPHSDVFRQAKDVAESDRGFSLAQKMVGRACGVKGIRPGAYCEPKMTSVGSQDTTGPMTRDELKDLACLGFSADLVMQSFCHTAAYPKPVDVNTHHTLPDFIMNRGGVSLRPGDGVIHSWLNRMLLPDTVGTGGDSHTRFPIGISFPAGSGLVAFAAATGVMPLDMPESVLVRFKGKMQPGITLRDLVHAIPLYAIKQGLLTVEKKGKKNIFSGRILEIEGLPDLKVEQAFELTDASAERSAAGCTIKLNKEPIIEYLNSNIVLLKWMIAEGYGDRRTLERRIQGMEKWLANPELLEADADAEYAAVIDIDLADIKEPILCAPNDPDDARPLSAVQGEKIDEVFIGSCMTNIGHFRAAGKLLDAHKGQLPTRLWVAPPTRMDAAQLTEEGYYSVFGKSGARIEIPGCSLCMGNQARVADGATVVSTSTRNFPNRLGTGANVFLASAELAAVAALIGKLPTPEEYQTYVAQVDKTAVDTYRYLNFNQLSQYTEKADGVIFQTAV

SEQ ID NO: 21 - DNA, 945 bp, Saccharomyces cerevisiae
  CDS (1)..(945)
atgccatctactaccaacactgctgctgctaacgtcattgaaaagaagcctgtttctttctccaacatcttgctaggtgcttgtttgaacttgtctgaagttaccactttaggtcaaccattggaagttgtcaagaccaccatggctgccaacagaaacttcactttcttggaatctgtcaagcacgtctggtcccgtggtggtattttgggttactaccaaggtttgattccatgggcttggattgaagcttccaccaagggtgccgtcttgttgttcgtttctgctgaagctgaataccgtttcaaatctttgggtttgaacaactttgcttctggtatcttaggtggtgttaccggtggtgtcactcaagcttacttgaccatgggtttctgtacttgtatgaaaactgtcgaaatcaccagacacaaatctgcttctgctggtggtgttccacaatcttcctggtccgttttcaagaacatctacaagaaggaaggtatcagaggtatcaacaagggtgtcaatgctgttgccatcagacaaatgactaactggggttccagattcggtttgtccagattggttgaagatggtatcagaaagatcactggtaagaccaacaaggacgacaaattgaacccattcgaaaagattggtgcttctgctttgggtggtggtttatctgcttggaaccaaccaattgaagtcatcagagttgaaatgcaatccaagaaggaagatccaaacagaccaaagaacttgaccgtcggtaagactttcaaatacatctaccaatctaacggtttgaagggtttatacagaggtgttactccaagaattggtttgggtatctggcaaaccgtctttatggttggtttcggtgacatggccaaggaattcgttgccagaatgaccggtgaaactccagttgccaagcactaa

SEQ ID NO: 22 - protein, 314 aa, Saccharomyces cerevisiae
MPSTTNTAAANVIEKKPVSFSNILLGACLNLSEVTTLGQPLEVVKTTMAANRNFTFLESVKHVWSRGGILGYYQGLIPWAWIEASTKGAVLLFVSAEAEYRFKSLGLNNFASGILGGVTGGVTQAYLTMGFCTCMKTVEITRHKSASAGGVPQSSWSVFKNIYKKEGIRGINKGVNAVAIRQMTNWGSRFGLSRLVEDGIRKITGKTNKDDKLNPFEKIGASALGGGLSAWNQPIEVIRVEMQSKKEDPNRPKNLTVGKTFKYIYQSNGLKGLYRGVTPRIGLGIWQTVFMVGFGDMAKEFVARMTGETPVAKH

SEQ ID NO: 23 - DNA, 975 bp, Saccharomyces cerevisiae
  CDS (1)..(975)
atgtcctctgacaactccaagcaagacaaacaaatcgaaaagactgctgctcaaaagatctccaaatttggttctttcgttgctggtggtttggctgcttgtatcgctgtcactgttaccaacccaattgaattgatcaagatcagaatgcaattgcaaggtgaaatgtctgcttctgctgccaaggtctacaagaacccaatccaaggtatggccgttatcttcaagaacgaaggtatcaagggtttgcaaaagggtttgaacgctgcttacatctaccaaattggtttgaacggttccagattaggtttctacgaaccaattagatcttctttgaaccaattattcttcccagaccaagaaccacacaaggtccaatctgttggtgttaacgtcttttccggtgctgcttccggtattatcggtgccgttatcggttctccattattcttggtcaagaccagattacaatcttactctgaattcatcaagattggtgaacaaacccactacactggtgtctggaacggtttagtcaccattttcaagactgaaggtgtcaagggtttgttcagaggtatcgatgctgccattttgagaaccggtgctggttcttccgttcaattgccaatctacaacactgccaagaacatcttggtcaagaacgatttgatgaaggacggtccagctctacatttgactgcttccaccatctctggtttgggtgttgccgttgttatgaacccatgggatgtcatcttgaccagaatttacaaccaaaagggtgacttgtacaagggtccaattgactgtttggtcaagactgttagaattgaaggtgtcactgctttgtacaagggtttcgctgctcaagttttcagaattgctcctcacaccatcatgtgtttgactttcatggaacaaaccatgaaattggtttactccattgaatctcgtgttttgggtcacaattaa

SEQ ID NO: 24 - protein, 324 aa, Saccharomyces cerevisiae
MSSDNSKQDKQIEKTAAQKISKFGSFVAGGLAACIAVTVTNPIELIKIRMQLQGEMSASAAKVYKNPIQGMAVIFKNEGIKGLQKGLNAAYIYQIGLNGSRLGFYEPIRSSLNQLFFPDQEPHKVQSVGVNVFSGAASGIIGAVIGSPLFLVKTRLQSYSEFIKIGEQTHYTGVWNGLVTIFKTEGVKGLFRGIDAAILRTGAGSSVQLPIYNTAKNILVKNDLMKDGPALHLTASTISGLGVAVVMNPWDVILTRIYNQKGDLYKGPIDCLVKTVRIEGVTALYKGFAAQVFRIAPHTIMCLTFMEQTMKLVYSIESRVLGHN

SEQ ID NO: 25 - DNA, 3543 bp, Saccharomyces cerevisiae
  CDS (1)..(3543)
atgtcctcttccaagatcttggctggtttgagagacaacttttctttgttgggtgaaaagaacaagattttggtcgccaacagaggtgaaatcccaatcagaattttcagatctgctcacgaattgtctatgagaactatcgccatctactctcacgaagatagattatccatgcacagattgaaggctgatgaagcctacgttatcggtgaagaaggtcaatacaccccagtcggtgcttacttggccatggacgaaatcatcgaaattgccaagaagcacaaggtcgatttcatccacccaggttacggtttcttgtctgaaaactctgaatttgctgacaaggttgttaaggctggtattacctggattggtccaccagctgaagtcattgaatctgttggtgacaaggtttctgccagacatttggctgctcgtgccaacgttccaactgtcccaggtactccaggtcctatcgaaaccgttcaagaagctctagatttcgtcaatgaatacggttacccagttatcatcaaggctgctttcggtggtggtggtcgtggtatgagagttgtcagagaaggtgacgatgtcgctgatgctttccaaagagccacttctgaagctagaactgctttcggtaacggtacttgtttcgtcgaaagattcttggacaagccaaagcacattgaagttcaattattagctgacaaccacggtaacgttgtccacttgttcgaaagagactgttccgtccaaagacgtcaccaaaaggttgtcgaagttgctccagctaagactttaccaagagaagttagagatgctatcttgaccgatgccgttaagttggctaaggtttgtggttacagaaacgctggtactgctgaattcttggttgacaaccaaaacagacattacttcattgaaatcaacccaagaattcaagtcgaacacaccatcactgaagaaatcactggtattgacattgtctccgctcaaatccaaatcgccgctggtgctactttgactcaattaggtctattacaagacaaaatcaccaccagaggtttctctatccaatgtcgtatcaccactgaagatccatccaagaacttccaaccagacactggtcgtttggaagtctacagatccgctggtggtaacggtgtcagattggacggtggtaacgcctacgctggtgctaccatctctccacactacgactccatgttggttaagtgttcctgttctggttctacctacgaaattgtcagaagaaagatgatcagagctttgattgaattcagaatcagaggtgtcaagaccaacatcccattcttgttgactttgttgaccaacccagttttcattgaaggtacctactggaccactttcatcgatgacactccacaattgttccaaatggtttcctctcaaaacagagctcaaaaattgttgcactacttggctgacttggccgtcaacggttcctctatcaagggtcaaatcggtttaccaaagttgaagtccaacccttccgttccacatttgcacgatgctcaaggtaatgtcatcaacgttaccaaatctgccccaccatccggttggagacaagtcttgttggaaaagggtccatccgaatttgccaagcaagtcagacaattcaacggtactttgttgatggacaccacctggagagatgctcaccaatctttgctagctaccagagtcagaactcacgatttggccaccattgctccaaccactgctcacgctttggctggtgcctttgctttggaatgttggggtggtgctactttcgatgtcgccatgagattcttgcatgaggacccatgggaaagattgagaaaattgagatctttggtcccaaacattccattccaaatgttgttgagaggtgctaacggtgttgcttactcctctttgccagacaacgccattgaccatttcgttaagcaagccaaggacaatggtgttgacattttcagagtctttgacgctttgaacgacttggaacaattgaaggttggtgttaatgctgtcaagaaggctggtggtgttgtcgaagctaccgtttgttactctggtgacatgttgcaaccaggtaagaaatacaacttggactactacttagaagttgtcgaaaagatcgttcaaatgggtactcacatcttgggtatcaaggacatggctggtaccatgaagccagctgctgccaaattgttgattggttctttacgtaccagatacccagacttgccaatccacgttcactctcatgactccgctggtactgctgttgcttccatgactgcttgtgctttggccggtgctgatgttgttgacgttgccattaactccatgtccggtttgacctctcaaccatctattaacgctttgttggcctccttggaaggtaacattgacactggtatcaacgtcgaacacgttagagaattggacgcttactgggctgaaatgagattattatactcttgtttcgaagctgacttgaagggtccagaccctgaagtttaccaacacgaaattccaggtggtcaattgaccaacttgttgttccaagctcaacaattaggtctaggtgaacaatgggctgaaaccaagagagcttacagagaagctaactacttgttgggtgacattgttaaggtcaccccaacttctaaggtcgttggtgatttggctcaattcatggtttctaacaaattgacttctgatgacatcagaagattagctaactctttggacttcccagactccgttatggacttcttcgaaggtttgatcggtcaaccatacggtggtttcccagaaccattgagatccgatgttttgagaaacaagcgtcgtaaattgacttgtagaccaggtttagaattggaaccattcgatttggaaaagatcagagaagatttgcaaaacagattcggtgatatcgatgaatgtgatgttgcctcctacaacatgtatcctcgtgtctacgaagatttccaaaagattagagaaacttacggtgacttgtctgtcttaccaaccaagaacttcttggctccagctgaaccagacgaagaaatcgaagtcaccattgaacaaggtaagactttgattatcaaattacaagctgttggtgatttgaacaagaaaaccggtcaaagagaagtctacttcgaattgaacggtgaattgagaaagatcagagttgctgacaaatctcaaaacattcaatctgttgccaagccaaaggctgatgtccacgacacccaccaaatcggtgctccaatggctggtgtcatcattgaagtcaaggttcacaagggttctttggtcaagaagggtgaatctatcgccgttttgtctgctatgaagatggaaatggttgtttcctctccagctgatggtcaagtcaaagatgtctttatccgtgacggtgaatccgtcgatgcttctgacttgttggttgttttggaagaagaaactctaccaccttctcaaaagaaataa

SEQ ID NO: 26 - protein, 1180 aa, Saccharomyces cerevisiae
MSSSKILAGLRDNFSLLGEKNKILVANRGEIPIRIFRSAHELSMRTIAIYSHEDRLSMHRLKADEAYVIGEEGQYTPVGAYLAMDEIIEIAKKHKVDFIHPGYGFLSENSEFADKVVKAGITWIGPPAEVIESVGDKVSARHLAARANVPTVPGTPGPIETVQEALDFVNEYGYPVIIKAAFGGGGRGMRVVREGDDVADAFQRATSEARTAFGNGTCFVERFLDKPKHIEVQLLADNHGNVVHLFERDCSVQRRHQKVVEVAPAKTLPREVRDAILTDAVKLAKVCGYRNAGTAEFLVDNQNRHYFIEINPRIQVEHTITEEITGIDIVSAQIQIAAGATLTQLGLLQDKITTRGFSIQCRITTEDPSKNFQPDTGRLEVYRSAGGNGVRLDGGNAYAGATISPHYDSMLVKCSCSGSTYEIVRRKMIRALIEFRIRGVKTNIPFLLTLLTNPVFIEGTYWTTFIDDTPQLFQMVSSQNRAQKLLHYLADLAVNGSSIKGQIGLPKLKSNPSVPHLHDAQGNVINVTKSAPPSGWRQVLLEKGPSEFAKQVRQFNGTLLMDTTWRDAHQSLLATRVRTHDLATIAPTTAHALAGAFALECWGGATFDVAMRFLHEDPWERLRKLRSLVPNIPFQMLLRGANGVAYSSLPDNAIDHFVKQAKDNGVDIFRVFDALNDLEQLKVGVNAVKKAGGVVEATVCYSGDMLQPGKKYNLDYYLEVVEKIVQMGTHILGIKDMAGTMKPAAAKLLIGSLRTRYPDLPIHVHSHDSAGTAVASMTACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTSDDIRRLANSLDFPDSVMDFFEGLIGQPYGGFPEPLRSDVLRNKRRKLTCRPGLELEPFDLEKIREDLQNRFGDIDECDVASYNMYPRVYEDFQKIRETYGDLSVLPTKNFLAPAEPDEEIEVTIEQGKTLIIKLQAVGDLNKKTGQREVYFELNGELRKIRVADKSQNIQSVAKPKADVHDTHQIGAPMAGVIIEVKVHKGSLVKKGESIAVLSAMKMEMVVSSPADGQVKDVFIRDGESVDASDLLVVLEEETLPPSQKK

SEQ ID NO: 27 - DNA, 1332 bp, Saccharomyces cerevisiae
  CDS (1)..(1332)
atgtcctctgcttctgaacaaactttgaaggaaagatttgctgaaatcattccagctaaggctgaagaaatcaagaaattcaagaaggaacacggtaagactgttatcggtgaagtcttgttggaacaagcttacggtggtatgagaggtatcaagggtttagtctgggaaggttctgttttggacccagaagaaggtatcagattccgtggtagaaccattccagaaatccaaagagaattgccaaaggctgaaggttccactgaaccattaccagaagctttgttctggttattgttgaccggtgaaattccaaccgatgctcaagtcaaggctttgtctgctgatttggctgcccgttctgaaatcccagaacacgttatccaattgttggactctctaccaaaggacttgcacccaatggctcaattctccattgctgttaccgccttggaatctgaatccaagttcgctaaggcctacgctcaaggtgtttccaagaaggaatactggtcctacaccttcgaagattctttggatttgttgggtaaattgcctgtcattgcttccaagatctacagaaacgttttcaaggacggtaagatcacttctactgacccaaacgctgactacggtaagaacttggctcaattgttgggttacgaaaacaaagatttcatcgatttgatgagattatacttgaccattcactctgaccacgaaggtggtaatgtctctgctcacactacccacttggtcggttctgctttgtcctctccatacttgtctttggctgccggtttgaacggtttggctggtcctttgcacggtagagctaaccaagaagtcttggaatggttattcaaattgagagaagaagtcaagggtgactactccaaggaaaccattgaaaaatacttatgggacactttgaacgccggtcgtgttgttccaggttacggtcatgccgttttgagaaagaccgatccaagatacactgcccaaagagaatttgctttgaagcatttcccagactacgaattattcaaattggtttccaccatctacgaagttgctccaggtgtcttgaccaagcacggtaagaccaagaacccatggccaaacgttgactctcactctggtgttttgctacaatactacggtttgactgaagcttctttctacactgtcttattcggtgttgccagagccattggtgtcttgccacaattgatcattgacagagctgttggtgctccaattgaaagaccaaagtctttctccactgaaaaatacaaggaattggtcaagaagatcgaatccaagaactaa

SEQ ID NO: 28 - protein, 443 aa, Saccharomyces cerevisiae
MSSASEQTLKERFAEIIPAKAEEIKKFKKEHGKTVIGEVLLEQAYGGMRGIKGLVWEGSVLDPEEGIRFRGRTIPEIQRELPKAEGSTEPLPEALFWLLLTGEIPTDAQVKALSADLAARSEIPEHVIQLLDSLPKDLHPMAQFSIAVTALESESKFAKAYAQGVSKKEYWSYTFEDSLDLLGKLPVIASKIYRNVFKDGKITSTDPNADYGKNLAQLLGYENKDFIDLMRLYLTIHSDHEGGNVSAHTTHLVGSALSSPYLSLAAGLNGLAGPLHGRANQEVLEWLFKLREEVKGDYSKETIEKYLWDTLNAGRVVPGYGHAVLRKTDPRYTAQREFALKHFPDYELFKLVSTIYEVAPGVLTKHGKTKNPWPNVDSHSGVLLQYYGLTEASFYTVLFGVARAIGVLPQLIIDRAVGAPIERPKSFSTEKYKELVKKIESKN

SEQ ID NO: 29 - DNA, 1317 bp, Sus scrofa
  CDS (1)..(1317)
atggcttcttctaccaacttgaaagatatcttggctgacttgattccaaaggaacaagccagaatcaagactttcagacaacaacacggtaacaccgttgtcggtcaaatcactgttgacatgatgtacggtggtatgagaggtatgaagggtttagtctacgaaacctctgttttggacccagacgaaggtatcagattcagaggttactccattccagaatgtcaaaagatgttgccaaaggctaagggtggtgaagaacctttgccagaaggtttattctggttattggttaccggtcaaatcccaactgaagaacaagtctcctggttatccaaggaatgggctaagcgtgctgctctaccatctcacgttgttaccatgttggacaacttcccaaccaacttgcacccaatgtcccaattgtctgctgccatcactgctttgaactctgaatctaactttgccagagcttatgctgaaggtattcaccgtaccaagtactgggaattgatctacgaagattgtatggacttgattgccaagttgccatgtgttgctgccaagatctacagaaacttatacagagaaggttcttccattggtgccattgactccaaattggactggtcccacaacttcaccaacatgttgggttacaccgatgctcaattcactgaattgatgagattatacttgaccattcactctgaccacgaaggtggtaatgtctctgctcacacttctcatttggttggttctgctttgtctgacccatacttgtctttcgctgctgctatgaacggtttggctggtccattgcacggtttggctaaccaagaagttttggtctggttgactcaattacaaaaggaagttggtaaggatgtctctgacgaaaaattgagagactacatctggaacactttgaactctggtcgtgttgttccaggttacggtcacgctgtcttgagaaagactgacccaagatacacctgtcaaagagaatttgctttgaagcatttgcctcacgatccaatgttcaaattggttgcccaattatacaagattgtcccaaacgttttgttggaacaaggtaaggccaagaacccatggccaaacgtcgatgctcactctggtgttttgctacaatactacggtatgactgaaatgaactactacactgtcttattcggtgtctccagagctttgggtgtcttggctcaattgatctggtccagagctttgggtttcccattggaaagaccaaagtccatgtccaccgatggtttgatcaaattggtcgattccaagtaa

SEQ ID NO: 30 - protein, 438 aa, Sus scrofa
MASSTNLKDILADLIPKEQARIKTFRQQHGNTVVGQITVDMMYGGMRGMKGLVYETSVLDPDEGIRFRGYSIPECQKMLPKAKGGEEPLPEGLFWLLVTGQIPTEEQVSWLSKEWAKRAALPSHVVTMLDNFPTNLHPMSQLSAAITALNSESNFARAYAEGIHRTKYWELIYEDCMDLIAKLPCVAAKIYRNLYREGSSIGAIDSKLDWSHNFTNMLGYTDAQFTELMRLYLTIHSDHEGGNVSAHTSHLVGSALSDPYLSFAAAMNGLAGPLHGLANQEVLVWLTQLQKEVGKDVSDEKLRDYIWNTLNSGRVVPGYGHAVLRKTDPRYTCQREFALKHLPHDPMFKLVAQLYKIVPNVLLEQGKAKNPWPNVDAHSGVLLQYYGMTEMNYYTVLFGVSRALGVLAQLIWSRALGFPLERPKSMSTDGLIKLVDSK

SEQ ID NO: 31 - DNA, 1284 bp, Escherichia coli
  CDS (1)..(1284)
atggctgacaccaaggccaagttgaccttgaacggtgacactgctgtcgaattggatgttttgaaaggtactttgggtcaagatgtcattgatatcagaactttgggttccaagggtgttttcaccttcgacccaggtttcacctctactgcttcttgtgaatccaagatcactttcatcgatggtgacgaaggtatcctattacacagaggtttcccaattgaccaattagctactgactccaactacttggaagtttgttacatcttgttgaatggtgaaaagccaactcaagaacaatacgacgaatttaaaaccaccgttaccagacacaccatgattcacgaacaaatcaccagattattccacgctttccgtcgtgactcccacccaatggctgtcatgtgtggtatcactggtgctttggctgctttctaccatgactctttggatgtcaacaacccaagacacagagaaattgccgctttcttgttgttgtccaagatgccaaccatggctgctatgtgttacaagtactccatcggtcaacctttcgtttacccaagaaacgatttgtcttacgccggtaacttcttgaacatgatgttctccactccatgtgaaccttacgaagttaacccaattttggaaagagccatggacagaatcttgatcttgcacgctgaccatgaacaaaacgcttctacttctactgttagaactgccggttcttctggtgctaacccatttgcttgtatcgctgctggtattgcttctttatggggtccagctcatggtggtgccaacgaagctgctttgaagatgttggaagaaatttcttctgtcaagcacattccagaatttgtcagaagagctaaggacaagaacgactctttcagattgatgggtttcggtcaccgtgtctacaagaactacgacccaagagctaccgtcatgagagaaacctgtcacgaagttttgaaggaattgggtaccaaggatgacttgttggaagttgccatggaattggaaaacattgctttgaacgacccatacttcatcgaaaagaaattgtacccaaacgtcgatttctactccggtatcatcttaaaggctatgggtattccatcttccatgttcaccgttatctttgctatggccagaactgttggttggatcgctcactggtccgaaatgcactctgatggtatgaagattgccagaccaagacaattatacactggttacgaaaagagagatttcaaatctgatatcaagagataa

SEQ ID NO: 32 - protein, 427 aa, Escherichia coli
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFLLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 33 - DNA, 1410 bp, Listeria innocua
  CDS (1)..(1410)
atggaatctttggaattggaacaattagtcaagaaggttttgttggaaaaattggctgaacaaaaggaagttccaaccaagaccaccacccaaggtgccaagtccggtgttttcgacaccgtcgatgaagctgtccaagctgctgtcattgctcaaaactgttacaaggaaaaatctttggaagaaagaagaaacgttgtcaaggccatcagagaagctttgtacccagaaatcgaaaccattgccaccagagctgttgctgaaaccggtatgggtaatgtcactgacaagatcttgaagaacactttggccatcgaaaagaccccaggtgttgaagatttgtacactgaagttgccactggtgacaacggtatgactttgtacgaattgtctccatacggtgtcatcggtgctgttgccccatctaccaacccaactgaaactttgatctgtaactccattggtatgttggctgctggtaatgctgttttctactctcctcacccaggtgccaagaacatctctttatggttaatcgaaaaattgaacaccattgtccgtgactcttgtggtatcgacaacttgattgtcactgttgccaagccttccatccaagctgctcaagaaatgatgaaccatccaaaggtcccattgttggttatcactggtggtccaggtgttgtcttgcaagctatgcaatctggtaagaaggtcattggtgctggtgctggtaacccaccatctatcgtcgatgaaactgctaacattgaaaaggctgccgctgatatcgttgacggtgcttctttcgaccacaacatcctatgtattgctgaaaaatccgttgttgccgttgactccattgctgatttcttattattccaaatggaaaagaacggtgctttgcacgttaccaacccatctgatatccaaaaattggaaaaggttgctgtcactgacaagggtgtcaccaacaagaaattggttggtaagtctgctactgaaatcttgaaggaagctggtattgcttgtgacttcactccaagattaatcattgtcgaaactgaaaagtcccacccatttgccaccgttgaattgttgatgccaattgtcccagttgtcagagttccagacttcgatgaagctttggaagttgccatcgaattggaacaaggtttgcaccacactgctaccatgcactctcaaaacatctccagattgaacaaggctgctagagacatgcaaacttccatctttgtcaagaacggtccatctttcgctggtttaggtttcagaggtgaaggttccactactttcaccattgctaccccaactggtgaaggtaccaccaccgctagacatttcgctagaagaagaagatgtgttttgactgatggtttctccatacgttaa

SEQ ID NO: 34 - protein, 469 aa, Listeria innocua
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRDSCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 35 - DNA, 2367 bp, Lactobacillus plantarum
  CDS (1)..(2367)
atgaccactgactactcttctccagcttacctacaaaaggtcgacaaatactggagagccgctaactacctatctgttggtcaattatacttgaaggactaccctttgttgcaacaaccattgaaggcttctgatgtcaaggtccacccaatctgtcactggggtaccattgctggtcaaaactccatctacgctcatttgaacagagtcatcaacaaatacggtttgaaaatgttctacgtcgaaggtcctggtcacggtggtcaagttatggtttccaactcttacttggatggtacttacactgatatctacccagaaatcactcaagatgtcgaaggtatgcaaaaattattcaagcaattctctttcccaggtggtgttgcttctcacgctgctccagaaaccccaggttccattcacgaaggtggtgaattgggttactccatctctcacggtgtcggtgccattttggacaacccagatgaaattgccgccgttgttgttggtgatggtgaatctgaaactggtccattagctacctcctggcaatctaccaaattcattaacccaattaacgacggtgccgtcttaccaattttgaacttgaacggtttcaagatctccaacccaaccattttcggtagaacttctgacgctaagatcaaggaatacttcgaatccatgtcttgggaaccaatcttcgtcgaaggtgatgacccagaaaaggtccatccagtcttggccaaggctatggacgaagctgttgaaaagatcaaggccatccaaaagcacgctagagaaaacgatgacgctactttgccagtctggccaatgattgtctttagagccccaaagggttggaccggtccaaagtcctgggacggtgacaagatcgaaggttctttcagagctcaccaaatcccaattccagttgaccaaaatgacatggaacacgctgatgctttggttgactggttggaatcctaccaaccaaaggaattgttcaacgaagatggttctttgaaggacgatatcaaggaaatcattccaactggtgactccagaatggctgctaacccaatcaccaacggtggtgttgacccaaaggctttgaacttgccaaacttcagagactatgctgtcgacacctccaaggaaggtgctaacgttaagcaagacatgttggtctggtctgactacttgcgtgacgttatcaagaagaacccagacaacttcagattgtttggtccagacgaaaccatgtccaacagattgtacggtgttttcgaaaccaccaacagacaatggatggaagatattcacccagattctgaccaatacgaagctgctgccggtagagttttggatgctcaattatctgaacaccaagctgaaggttggttagaaggttacgttttgactggtcgtcacggtttgtttgcttcttacgaagctttcttgagagttgtcgactccatgttgactcaacatttcaaatggttaagaaaggctaacgaattggactggagaaagaaatacccatctttgaacattattgctgcctccaccgttttccaacaagatcacaacggttacactcaccaagatcctggtgccttgacccacttggctgaaaagaagccagaatacatcagagaatacttgccagctgatgctaacactttgttggctgtcggtgatgttatcttcagatctcaagaaaagatcaactacgttgttacctctaagcatccaagacaacaatggttctccattgaagaagccaagcaattggttgacaacggtttgggtatcatcgactgggcttctactgaccaaggttctgaaccagacattgttttcgctgctgctggtactgaaccaactttggaaactttggctgccatccaattgttgcacgactccttcccagaaatgaagatcagattcgtcaatgttgtcgatattttgaaattgagatctccagaaaaggacccaagaggtctatctgatgctgaatttgaccattacttcaccaaggacaagcctgttgttttcgctttccacggttacgaagatttggtcagagatatcttctttgacagacacaaccacaacttatacgtccacggttacagagaaaacggtgatatcaccactccattcgatgtccgtgttatgaaccaaatggaccgtttcgacttggccaagaccgccattgctgctcaaccagctatggaaaacactggtgctgctttcgttcaatccatggacaacatgttggccaagcacaacgcttacatcagagatgctggtaccgatttgccagaagtcaatgactggcaatggaaaggtcttaagtaa

SEQ ID NO: 36 - protein, 788 aa, Lactobacillus plantarum
MTTDYSSPAYLQKVDKYWRAANYLSVGQLYLKDYPLLQQPLKASDVKVHPICHWGTIAGQNSIYAHLNRVINKYGLKMFYVEGPGHGGQVMVSNSYLDGTYTDIYPEITQDVEGMQKLFKQFSFPGGVASHAAPETPGSIHEGGELGYSISHGVGAILDNPDEIAAVVVGDGESETGPLATSWQSTKFINPINDGAVLPILNLNGFKISNPTIFGRTSDAKIKEYFESMSWEPIFVEGDDPEKVHPVLAKAMDEAVEKIKAIQKHARENDDATLPVWPMIVFRAPKGWTGPKSWDGDKIEGSFRAHQIPIPVDQNDMEHADALVDWLESYQPKELFNEDGSLKDDIKEIIPTGDSRMAANPITNGGVDPKALNLPNFRDYAVDTSKEGANVKQDMLVWSDYLRDVIKKNPDNFRLFGPDETMSNRLYGVFETTNRQWMEDIHPDSDQYEAAAGRVLDAQLSEHQAEGWLEGYVLTGRHGLFASYEAFLRVVDSMLTQHFKWLRKANELDWRKKYPSLNIIAASTVFQQDHNGYTHQDPGALTHLAEKKPEYIREYLPADANTLLAVGDVIFRSQEKINYVVTSKHPRQQWFSIEEAKQLVDNGLGIIDWASTDQGSEPDIVFAAAGTEPTLETLAAIQLLHDSFPEMKIRFVNVVDILKLRSPEKDPRGLSDAEFDHYFTKDKPVVFAFHGYEDLVRDIFFDRHNHNLYVHGYRENGDITTPFDVRVMNQMDRFDLAKTAIAAQPAMENTGAAFVQSMDNMLAKHNAYIRDAGTDLPEVNDWQWKGLK

SEQ ID NO: 37 - DNA, 2478 bp, Bifidobacterium animalis
  CDS (1)..(2478)
atgaccaaccctgtcattggtaccccatggcaaaagttggacagacctgtttctgaagaagctatcgaaggtatggacaaatactggagagttgccaactacatgtctattggtcaaatctacttgagatccaatccattaatgaaggaaccattcaccagagatgatgtcaagcacagattagtcggtcactggggtaccaccccaggtttaaacttcttgttggctcacatcaacagattgattgctgaccaccaacaaaacaccgttttcatcatgggtccaggtcacggtggtccagctggtactgctcaatcctacattgacggtacctacactgaatactacccaaacatcactaaggatgaagctggtctacaaaagttcttcagacaattctcttacccaggtggtatcccatctcacttcgctccagaaactccaggttccattcacgaaggtggtgaattgggttacgccttatctcacgcttacggtgccatcatggacaacccatctttattcgttccatgtattattggtgacggtgaagctgaaactggtccattagctaccggttggcaatctaacaaattagtcaacccaagaactgatggtattgttttgccaattttgcacttgaacggttacaagattgctaacccaactatcttggccagaatttctgacgaagaattgcacgacttcttcagaggtatgggttaccatccatacgaatttgttgccggtttcgacaacgaagatcatttgtccattcacagaagatttgctgaattgtttgaaaccattttcgatgaaatctgtgacatcaaggctgctgctcaaaccgatgacatgactagacctttctacccaatgttgatcttcagaaccccaaagggttggacctgtccaaagttcatcgatggtaagaaaactgaaggttcctggagagcccaccaagtcccattggcctccgctcgtgacactgaagctcatttcgaagttttgaagggttggatggaatcttacaagccagaagaattgttcaacgctgacggttccatcaaggaagatgtcactgctttcatgccaaagggtgaattgagaattggtgccaacccaaacgccaacggtggtagaatccgtgaagatttgaagttgccagaattggaccaatacgaaatcactggtgttaaggaatacggtcacggttggggtcaagttgaagccccaagatctctaggtgcttactgtagagatatcatcaagaacaacccagactctttcagagttttcggtccagacgaaactgcttccaacagattgaatgctacctacgaagtcaccaagaagcaatgggacaacggttacttgtctgctttggttgacgaaaacatggccgttactggtcaagttgtcgaacaattgtctgaacaccaatgtgaaggtttcttggaagcttacttgttgactggtcgtcacggtatctggtcctcttacgaatccttcgttcatgtcattgattccatgttgaaccaacatgccaaatggttggaagctactgtcagagaaatcccatggagaaagcctatctcctccgtcaacttattagtctcctctcacgtctggagacaagaccacaacggtttctctcaccaagatccaggtgttacctctgttttgttgaacaagactttcaacaacgaccacgttaccaacatttactttgctaccgatgccaacatgttgttggccattgctgaaaaatgtttcaaatccactaacaagattaacgccatcttcgctggtaagcaaccagctgctacctggatcactttggacgaagtcagagctgaattggaagctggtgctgctgaatggaaatgggcttccaatgctaagtctaacgacgaagttcaagttgttttggctgccgctggtgatgtcccaactcaagaaatcatggctgcttctgatgctttgaacaagatgggtatcaagttcaaggttgtcaacgttgtcgatttgatcaagttgcaatcttctaaggaaaacgatgaagctatgtctgacgaagatttcgccgatttgttcaccgctgacaagccagttttgtttgcttaccactcttatgctcaagatgtcagaggtttgatctacgacagaccaaaccacgacaacttcactgttgttggttacaaggaacaaggttccaccaccaccccattcgacatggtccgtgtcaacgacatggaccgttacgctttacaagctaaggctttggaattgattgacgctgacaaatacgctgacaagatcaacgaattgaacgaattcagaaagaccgctttccaatttgctgtcgacaacggttacgatatcccagaattcaccgactgggtttacccagatgtcaaggttgacgaaacttctatgttgtctgctactgctgccactgctggtgacaatgaataa

SEQ ID NO: 38 - protein, 825 aa, Bifidobacterium animalis
MTNPVIGTPWQKLDRPVSEEAIEGMDKYWRVANYMSIGQIYLRSNPLMKEPFTRDDVKHRLVGHWGTTPGLNFLLAHINRLIADHQQNTVFIMGPGHGGPAGTAQSYIDGTYTEYYPNITKDEAGLQKFFRQFSYPGGIPSHFAPETPGSIHEGGELGYALSHAYGAIMDNPSLFVPCIIGDGEAETGPLATGWQSNKLVNPRTDGIVLPILHLNGYKIANPTILARISDEELHDFFRGMGYHPYEFVAGFDNEDHLSIHRRFAELFETIFDEICDIKAAAQTDDMTRPFYPMLIFRTPKGWTCPKFIDGKKTEGSWRAHQVPLASARDTEAHFEVLKGWMESYKPEELFNADGSIKEDVTAFMPKGELRIGANPNANGGRIREDLKLPELDQYEITGVKEYGHGWGQVEAPRSLGAYCRDIIKNNPDSFRVFGPDETASNRLNATYEVTKKQWDNGYLSALVDENMAVTGQVVEQLSEHQCEGFLEAYLLTGRHGIWSSYESFVHVIDSMLNQHAKWLEATVREIPWRKPISSVNLLVSSHVWRQDHNGFSHQDPGVTSVLLNKTFNNDHVTNIYFATDANMLLAIAEKCFKSTNKINAIFAGKQPAATWITLDEVRAELEAGAAEWKWASNAKSNDEVQVVLAAAGDVPTQEIMAASDALNKMGIKFKVVNVVDLIKLQSSKENDEAMSDEDFADLFTADKPVLFAYHSYAQDVRGLIYDRPNHDNFTVVGYKEQGSTTTPFDMVRVNDMDRYALQAKALELIDADKYADKINELNEFRKTAFQFAVDNGYDIPEFTDWVYPDVKVDETSMLSATAATAGDNE

SEQ ID NO: 39 - DNA, 1203 bp, Escherichia coli
  CDS (1)..(1203)
atgtcctccaagttggttttggttttgaactgtggttcttcttctttgaaatttgccatcattgatgctgtcaacggtgaagaatacttgtccggtttggctgaatgtttccatttgccagaagccagaatcaaatggaagatggacggtaacaagcaagaagctgctttgggtgctggtgctgctcactctgaagctttgaactttattgtcaacaccattttggctcaaaagccagaattgtctgctcaattgactgccatcggtcacagaattgtccacggtggtgaaaaatacacttcttccgttgtcattgacgaatccgttatccaaggtatcaaggatgctgcttctttcgctccattgcacaacccagctcatttgattggtattgaagaagctttgaaatctttcccacaattgaaggacaagaacgttgccgttttcgacactgctttccaccaaaccatgccagaagaatcttacttgtacgctttgccatacaacttatacaaggaacacggtatcagaagatacggtgctcacggtacttctcacttctacgtcactcaagaagctgccaagatgttgaacaagcctgtcgaagaattgaacatcatcacttgtcacttgggtaacggtggttccgtttctgccatcagaaacggtaagtgtgttgacacttccatgggtttgaccccattggaaggtttagtcatgggtaccagatctggtgacattgacccagccatcattttccatttgcacgacactttaggtatgtccgtcgatgctatcaacaagttgttgaccaaggaatctggtctattaggtttgactgaagttacctccgactgtcgttacgttgaagataactacgctaccaaggaagatgctaagagagctatggacgtttactgtcacagattggccaagtacatcggtgcttacactgctttgatggacggtagattagatgctgttgttttcaccggtggtatcggtgaaaacgctgccatggtcagagaattgtctctaggtaagttgggtgtcttaggtttcgaagttgaccacgaaagaaacttggctgcccgtttcggtaagtctggtttcatcaacaaggaaggtaccagaccagctgttgtcatcccaaccaatgaagaattggtcattgctcaagatgcttccagattgaccgcttaa

SEQ ID NO: 40 - protein, 400 aa, Escherichia coli
MSSKLVLVLNCGSSSLKFAIIDAVNGEEYLSGLAECFHLPEARIKWKMDGNKQEAALGAGAAHSEALNFIVNTILAQKPELSAQLTAIGHRIVHGGEKYTSSVVIDESVIQGIKDAASFAPLHNPAHLIGIEEALKSFPQLKDKNVAVFDTAFHQTMPEESYLYALPYNLYKEHGIRRYGAHGTSHFYVTQEAAKMLNKPVEELNIITCHLGNGGSVSAIRNGKCVDTSMGLTPLEGLVMGTRSGDIDPAIIFHLHDTLGMSVDAINKLLTKESGLLGLTEVTSDCRYVEDNYATKEDAKRAMDVYCHRLAKYIGAYTALMDGRLDAVVFTGGIGENAAMVRELSLGKLGVLGFEVDHERNLAARFGKSGFINKEGTRPAVVIPTNEELVIAQDASRLTA

SEQ ID NO: 41 - DNA, 2145 bp, Salmonella enterica
  CDS (1)..(2145)
atgtccagaatcatcatgttgattccaactggtacttccgtcggtttgacttctgtctctttgggtgttatcagagccatggaaagaaagggtgtcagattatctgtctttaaaccaattgctcaaccaagagccggtggtgacgctccagaccaaaccaccaccattgtcagagctaactccactctaccagctgctgaaccattgaagatgtctcacgttgaatccttgttgtcctctaaccaaaaggatgtcttgatggaagaaatcattgctaactaccatgccaacaccaaagatgctgaagttgttttggttgaaggtttagtcccaaccagaaagcaccaatttgctcaatctttgaactacgaaattgccaagactttaaacgctgaaatcgttttcgttatgtcccaaggtactgacaccccagaacaattgaacgaaagaatcgaattgaccagatcttctttcggtggtgccaagaacaccaacatcactggtgttatcatcaacaaattgaacgctccagtcgacgaacaaggtagaaccagaccagatttgtctgaaatcttcgatgactcctccaaggctcaagtcatcaagattgacccagctaaattacaagaatcctctccattgccagtcttaggtgccgttccatggtctttcgacttgattgctaccagagctatcgacatggccagacatttgaatgctaccatcatcaacgaaggtgacatcaagaccagacacgttaagtctgttactttctgtgccagatccattccacacatgttggaacacttcagagccggttctttgttggtcacttctgctgacagaccagatgtcttggttgctgcctgtttggctgccatgaacggtgttgaaatcggtgctttgttgttgaccggtggttacgaaatggatgctcgtatctccaagttgtgtgaaagagctttcgctactggtttgccagttttcatggtcaacactaacacctggcaaacctctctatctctacaatctttcaacttggaagttccagtcgatgaccacgaaagaattgaaaaggttcaagaatacgttgccaactacgtcaatgctgaatggattgaatctttgactgctacttctgaaagatccagaagattatctccaccagccttcagataccaattgactgaattggctagaaaggctggtaagcgtgtcgttttgccagaaggtgacgaaccaagaaccgtcaaggctgctgctatctgtgctgaacgtggtattgctacttgtgtcttattgggtaacccagacgaaatcaacagagttgccgcttctcaaggtgttgaattaggtgctggtattgaaattgttgacccagaagttgttagagaatcttacgttgctagattagtcgaattgagaaagtccaagggtatgactgaacctgttgctcgtgaacaattggaagataacgttgtcttgggtactttgatgttggaacaagatgaagtcgacggtttggtttccggtgctgtccacaccactgctaacaccatcagacctcctttgcaattgatcaagaccgctccaggttcctctttggtttcctctgttttcttcatgttgttgccagaacaagtttacgtctacggtgactgtgccatcaacccagacccaaccgctgaacaattagctgaaattgccattcaatctgctgactctgccattgctttcggtatcgaaccaagagttgctatgttgtcttactccactggtacttctggtgctggttctgatgtcgaaaaggttagagaagctaccagattggctcaagaaaagcgtccagacttgatgatcgatggtccattgcaatacgatgctgctgtcatggctgacgttgccaagtccaaggctccaaactctccagttgctggtagagctactgttttcatcttcccagacttgaacactggtaacaccacctacaaggctgtccaacgttctgctgatttgatttccatcggtccaatgttgcaaggtatgagaaagcctgtcaacgacttgtccagaggtgctttggtcgatgatatcgtctacaccattgccttgactgctatccaagcttcccaacaacagcagtaa

SEQ ID NO: 42 - protein, 714 aa, Salmonella enterica
MSRIIMLIPTGTSVGLTSVSLGVIRAMERKGVRLSVFKPIAQPRAGGDAPDQTTTIVRANSTLPAAEPLKMSHVESLLSSNQKDVLMEEIIANYHANTKDAEVVLVEGLVPTRKHQFAQSLNYEIAKTLNAEIVFVMSQGTDTPEQLNERIELTRSSFGGAKNTNITGVIINKLNAPVDEQGRTRPDLSEIFDDSSKAQVIKIDPAKLQESSPLPVLGAVPWSFDLIATRAIDMARHLNATIINEGDIKTRHVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYVNAEWIESLTATSERSRRLSPPAFRYQLTELARKAGKRVVLPEGDEPRTVKAAAICAERGIATCVLLGNPDEINRVAASQGVELGAGIEIVDPEVVRESYVARLVELRKSKGMTEPVAREQLEDNVVLGTLMLEQDEVDGLVSGAVHTTANTIRPPLQLIKTAPGSSLVSSVFFMLLPEQVYVYGDCAINPDPTAEQLAEIAIQSADSAIAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQASQQQQ

SEQ ID NO: 43 - DNA, 1017 bp, Salmonella enterica
  CDS (1)..(1017)
atgatcattgaaagagccagagaattggctgtcagagctccagcccgtgttgtctttcctgatgctttggacgaacgtgtcttgaaggctgctcattacttgcaacaatacggtttggccagaccagtcttggttgcttctccattcgctttgagacaatttgctctatcccacagaatggccatggacggtattcaagtcattgaccctcactctaacttgtccatgagacaaagattcgctcaaagatggttagccagagctggtgaaaagaccccaccagatgctgttgaaaaattgtctgacccattgatgttcgctgctgccatggtttctgccggtgaagctgatgtctgtattgctggtaacttgtcctccactgctaacgttttgagagctggtttgagagttatcggtttgcaaccaggttgtaagactctatcctctatcttcttgatgttgccacaatacgctggtccagctttgggtttcgctgactgttccgttgtcccacaaccaaccgctgctcaattggctgatatcgctttggcttctgctgacacctggagagccatcaccggtgaagaaccaagagttgccatgttgtctttctcttccaacggttctgcccgtcacccaaacgttgccaacgtccaacaagctactgaattggtcagagaaagagctccacaattattggttgacggtgaattgcaattcgatgctgctttcgttccagaagttgctgctcaaaaggctccagactctccattacaaggtagagccaacgtcatgatcttcccatctttggaagctggtaacatcggttacaagatcactcaaagattaggtggttacagagctgtcggtccattgattcaaggtttggctgctccattgcacgacttgtcccgtggttgttctgtccaagaaatcattgaattggctttggttgccgctgttccaagacaagctgatgtttccagagaaagatctttgcacactttagtagagtaa

SEQ ID NO: 44 - protein, 338 aa, Salmonella enterica
MIIERARELAVRAPARVVFPDALDERVLKAAHYLQQYGLARPVLVASPFALRQFALSHRMAMDGIQVIDPHSNLSMRQRFAQRWLARAGEKTPPDAVEKLSDPLMFAAAMVSAGEADVCIAGNLSSTANVLRAGLRVIGLQPGCKTLSSIFLMLPQYAGPALGFADCSVVPQPTAAQLADIALASADTWRAITGEEPRVAMLSFSSNGSARHPNVANVQQATELVRERAPQLLVDGELQFDAAFVPEVAAQKAPDSPLQGRANVMIFPSLEAGNIGYKITQRLGGYRAVGPLIQGLAAPLHDLSRGCSVQEIIELALVAAVPRQADVSRERSLHTLVE

SEQ ID NO: 45 - DNA, 972 bp, Bacillus subtilis
  CDS (1)..(972)
atggctgatttattctccaccgttcaagaaaaggttgctggtaaggacgtcaaaatcgttttcccagaaggtttggacgaaagaattttggaagctgtttccaaattggctggtaacaaggtcttgaacccaattgtcattggtaacgaaaacgaaatccaagctaaggccaaggaattgaacttgactttaggtggtgtcaagatctacgaccctcacacctacgaaggtatggaagatttggttcaagctttcgttgaaagaagaaagggtaaggctactgaagaacaagccagaaaggctttgttggacgaaaactacttcggtaccatgttggtctacaagggtttggctgatggtttggtttccggtgctgctcactccactgctgataccgtcagaccagctttgcaaatcatcaagaccaaggaaggtgtcaagaaaacctctggtgttttcatcatggccagaggtgaagaacaatacgtctttgctgactgtgccatcaacattgctccagactctcaagacttggctgaaattgccattgaatctgccaacactgccaagatgttcgatatcgaaccaagagttgccatgttgtctttctccaccaaaggttctgccaaatctgacgaaactgaaaaggttgctgacgctgtcaagatcgccaaggaaaaggctccagaattgactttggacggtgaattccaattcgatgctgctttcgttccatctgttgctgaaaagaaggctccagactctgaaatcaagggtgacgctaacgttttcgttttcccatctttggaagctggtaacattggttacaagattgctcaaagattaggtaactttgaagctgtcggtccaatcttacaaggtttgaacatgccagtcaacgatttgtcccgtggttgtaatgctgaagatgtctacaacttggctttgatcactgctgctcaagctctataa

SEQ ID NO: 46 - protein, 323 aa, Bacillus subtilis
MADLFSTVQEKVAGKDVKIVFPEGLDERILEAVSKLAGNKVLNPIVIGNENEIQAKAKELNLTLGGVKIYDPHTYEGMEDLVQAFVERRKGKATEEQARKALLDENYFGTMLVYKGLADGLVSGAAHSTADTVRPALQIIKTKEGVKKTSGVFIMARGEEQYVFADCAINIAPDSQDLAEIAIESANTAKMFDIEPRVAMLSFSTKGSAKSDETEKVADAVKIAKEKAPELTLDGEFQFDAAFVPSVAEKKAPDSEIKGDANVFVFPSLEAGNIGYKIAQRLGNFEAVGPILQGLNMPVNDLSRGCNAEDVYNLALITAAQAL

SEQ ID NO: 47 - DNA, 906 bp, Aspergillus terreus
  CDS (1)..(906)
atggaatccaaggttcaaaccaacgttccattaccaaaggctccattgactcaaaaggcccgtggtaagagaaccaaaggtattccagctttggttgctggtgcttgtgccggtgccgttgaaatctccattacctacccatttgaatctgccaagaccagagctcaattgaagagaagaaaccacgatgttgctgccatcaagccaggtatcagaggttggtacgctggttacggtgccactttagtcggtaccactttgaaggcttctgttcaatttgcttctttcaacatctacagatctgctttgtctggtccaaacggtgaattgtccactggtgcttccgttttggctggtttcggtgctggtgtcactgaagctgtcttggctgtcactccagctgaagctatcaagaccaagatcattgacgctagaaaggttggtaacgctgaattgtccaccactttcggtgccattgctggtatcttacgtgacagaggtccattaggtttcttctctgctgtcggtccaaccatcttgagacaatcttctaacgctgctgtcaaattcaccgtctacaacgaattgattggtttggccagaaagtactccaagaacggtgaagatgtccacccattggcttccactttggtcggttctgttaccggtgtttgttgtgcttggtccactcaacctttggacgttatcaagaccagaatgcaatctttgcaagctcgtcaattgtacggtaacactttcaactgtgtcaagactttgttgagaaacgaaggtattggtgttttctggtctggtgtctggttcagaaccggtagattatctttgacctctgccatcatgttcccagtttacgaaaaggtttacaaattcttgactcaaccaaattaa

SEQ ID NO: 48 - protein, 301 aa, Aspergillus terreus
MESKVQTNVPLPKAPLTQKARGKRTKGIPALVAGACAGAVEISITYPFESAKTRAQLKRRNHDVAAIKPGIRGWYAGYGATLVGTTLKASVQFASFNIYRSALSGPNGELSTGASVLAGFGAGVTEAVLAVTPAEAIKTKIIDARKVGNAELSTTFGAIAGILRDRGPLGFFSAVGPTILRQSSNAAVKFTVYNELIGLARKYSKNGEDVHPLASTLVGSVTGVCCAWSTQPLDVIKTRMQSLQARQLYGNTFNCVKTLLRNEGIGVFWSGVWFRTGRLSLTSAIMFPVYEKVYKFLTQPN

SEQ ID NO: 49 - DNA, 600 bp, Saccharomyces cerevisiae
aacatatatacacaattacagtaacaataacaagaggacagatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttctttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaaca

SEQ ID NO: 50 - DNA, 600 bp, Saccharomyces cerevisiae
ttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 51 - DNA, 600 bp, Saccharomyces cerevisiae
ttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 52 - DNA, 600 bp, Saccharomyces cerevisiae
gtgtcgacgctgcgggtatagaaagggttctttactctatagtacctcctcgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 53 - DNA, 600 bp, Saccharomyces cerevisiae
agagaattttgccatcggacatgctaccttacgcttatatctctcattggaatatcgttttctgattaaaacacggaagtaagaacttaattcgtttttcgttgaactatgttgtgccagcgtaacattaaaaaagagtgtacaaggccacgttctgtcaccgtcagaaaaatatgtcaatgaggcaagaaccgggatggtaacaaaaatcacgatctgggtgggtgtgggtgtattggattataggaagccacgcgctcaacctggaattacaggaagctggtaattttttgggtttgcaatcatcaccatctgcacgttgttataatgtcccgtgtctatatatatccattgacggtattctatttttttgctattgaaatgagcgttttttgttactacaattggttttacagacggaattttccctatttgtttcgtcccatttttccttttctcattgttctcatatcttaaaaaggtcctttcttcataatcaatgctttcttttacttaatattttacttgcattcagtgaattttaatacatattcctctagtcttgcaaaatcgatttagaatcaagataccagcctaaaa

SEQ ID NO: 54 - DNA, 600 bp, Saccharomyces cerevisiae
ctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaa

SEQ ID NO: 55 - DNA, 600 bp, Saccharomyces cerevisiae
gggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 56 - DNA, 600 bp, Saccharomyces cerevisiae
caaacattaatttgttctgcatactttgaacctttcagaaaataaaaaacattacgcgcatacttaccctgctcgcgaagaagagtaacactaacgcattctatgggcaattgaagacagtattcagtacaagacatagtccgtttccttgagtcaattcctatagcattatgaactagccgcctttaagagtgccaagctgttcaacaccgatcatttttgatgatttggcgtttttgttatattgatagatttcttttgaattttgtcattttcacttttccactcgcaacggaatccggtggcaaaaaagggaaaagcattgaaatgcaatctttaacagtattttaaacaagttgcgacacggtgtacaattacgataagaattgctacttcaaagtacacacagaaagttaacatgaatggaattcaagtggacatcaatcgtttgaaaaagggcgaagtcagtttaggtacctcaatgtatgtatataagaatttttcctcccactttattgtttctaaaagttcaatgaagtaaagtctcaattggccttattactaactaataggtatcttataatcacctaataaaataga

SEQ ID NO: 57 - DNA, 600 bp, Saccharomyces cerevisiae
cagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaa

SEQ ID NO: 58 - DNA, 301 bp, Saccharomyces cerevisiae
agcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaa

SEQ ID NO: 59 - DNA, 301 bp, Saccharomyces cerevisiae
aataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctt

SEQ ID NO: 60 - DNA, 301 bp, Saccharomyces cerevisiae
agcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaaca

SEQ ID NO: 61 - DNA, 301 bp, Saccharomyces cerevisiae
aggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtc

SEQ ID NO: 62 - DNA, 301 bp, Saccharomyces cerevisiae
agtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggat

SEQ ID NO: 63 - DNA, 301 bp, Saccharomyces cerevisiae
agtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaata

SEQ ID NO: 64 - DNA, 301 bp, Saccharomyces cerevisiae
agattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactca

SEQ ID NO: 65 - protein, 252 aa, Escherichia coli
MSDWNPSLYLHFSAERSRPAVELLARVPLENVEYVADLGCGPGNSTALLQQRWPAARITGIDSSPAMIAEARSALPDCQFVEADIRNWQPVQALDLIFANASLQWLPDHYELFPHLVSLLNPQGVLAVQMPDNWLEPTHVLMREVAWEQNYPDRGREPLAGVHAYYDILSEAGCEVDIWRTTYYHQMPSHQAIIDWVTATGLRPWLQDLTESEQQLFLKRYHQMLEEQYPLQENGQILLAFPRLFIVARRME

SEQ ID NO: 66 - protein, 299 aa, Saccharomyces cerevisiae
MSTFSASDFNSERYSSSRPSYPSDFYKMIDEYHDGERKLLVDVGCGPGTATLQMAQELKPFEQIIGSDLSATMIKTAEVIKEGSPDTYKNVSFKISSSDDFKFLGADSVDKQKIDMITAVECAHWFDFEKFQRSAYANLRKDGTIAIWGYADPIFPDYPEFDDLMIEVPYGKQGLGPYWEQPGRSRLRNMLKDSHLDPELFHDIQVSYFCAEDVRDKVKLHQHTKKPLLIRKQVTLVEFADYVRTWSAYHQWKQDPKNKDKEDVADWFIKESLRRRPELSTNTKIEVVWNTFYKLGKRV

SEQ ID NO: 67 - protein, 178 aa, Brucella ceti str. Cudo
MPEVGGKTIEVLFSPDEIAKRNLELATIIAERKFHNLLTISILKGSFIFAADLIRAMHDAGVEPDVEFITMSSYGKGTTSTEVRLLRDIDSDVRDRDVLLIDDILESGKTLKFVRELMLERGARSVSIAVLLDKSMRRKVDLDADFVAFECPDYFVVGYGMDVGHAFRQLPYVGRVME

SEQ ID NO: 68 - DNA, 756 bp, Artificial sequence
  CpO sequence for Echerichia coli K12
atgtctgactggaacccatctttgtacttgcacttctccgctgaaagatccagaccagctgtcgaattgttggccagagttccattggaaaacgtcgaatacgttgctgacttgggttgtggtccaggtaactccactgctttgttgcaacaaagatggccagctgccagaatcactggtattgactcttccccagccatgattgctgaagctcgttctgctttgccagactgtcaattcgttgaagctgatatcagaaactggcaaccagtccaagctttggatttgatctttgccaatgcttctttgcaatggttaccagaccactacgaattgttccctcacttggtttccttgttgaaccctcaaggtgtcttggctgtccaaatgccagacaactggttggaaccaactcacgttttgatgagagaagttgcttgggaacaaaactacccagacagaggtagagaaccattggctggtgtccacgcttactacgatatcttatctgaagccggttgtgaagtcgatatctggagaaccacctactaccatcaaatgccatctcaccaagctatcattgactgggttaccgctaccggtctaagaccatggttacaagatttgactgaatctgaacaacaattattcttgaagcgttaccaccaaatgttggaagaacaatacccattgcaagaaaacggtcaaatcttgttggctttcccaagattattcattgttgccagaagaatggaa

SEQ ID NO: 69 - DNA, 897 bp, Artificial sequence
  CpO sequence for S. cerevisiae
atgtccactttctccgcttctgatttcaactctgaaagatactcctcttccagaccatcttacccatctgatttctacaagatgattgatgaataccacgatggtgaaagaaagttgttggtcgatgtcggttgtggtccaggtactgctactttacaaatggctcaagaattgaaaccatttgaacaaatcattggttctgacttgtctgctaccatgatcaagaccgctgaagttatcaaggaaggttctccagacacctacaagaacgtttctttcaagatttcctcttctgatgacttcaaattcttgggtgctgactccgttgacaagcaaaagattgacatgatcactgctgtcgaatgtgcccactggttcgacttcgaaaaattccaaagatctgcctacgctaacttgagaaaggacggtactattgccatctggggttacgctgacccaatcttcccagactacccagaattcgatgacttgatgatcgaagttccatacggtaagcaaggtttaggtccttactgggaacaaccaggtagatccagattgagaaacatgttgaaggactctcatttggatccagaattgttccacgatatccaagtttcctacttctgtgctgaagatgtccgtgacaaggtcaaattgcaccaacacaccaagaagccattattgatcagaaagcaagtcactttggttgaatttgctgactacgttagaacctggtccgcttaccaccaatggaagcaagacccaaagaacaaggacaaggaagatgttgccgactggttcatcaaggaatctttgagaagaagaccagaactatccaccaacaccaagattgaagttgtctggaacactttctacaaattgggtaagcgtgtg

SEQ ID NO: 70 - DNA, 534 bp, Artificial sequence
  CpO sequence for Brucella ceti str. Cudo
atgccagaagttggtggtaagaccattgaagtcttattctctccagacgaaattgccaagagaaacttggaattggccaccattattgctgaaagaaagttccacaacttgttgactatctccatcttgaagggttctttcatctttgctgctgacttgatcagagccatgcacgatgctggtgttgaaccagatgtcgaattcatcaccatgtcctcttacggtaagggtactacctctactgaagtcagattactaagagatatcgactctgatgtcagagacagagatgtcttgttgatcgatgacatcttggaatctggtaagactttgaaattcgttagagaattgatgttggaaagaggtgctcgttctgtttccattgctgtcttattggacaagtccatgagaagaaaggttgacttggatgctgacttcgttgctttcgaatgtccagactacttcgttgttggttacggtatggacgtcggtcacgctttcagacaattgccatacgttggtcgtgtcatggaa